(19) United States
(12) Patent Application Publication
(10) Pub. No.: US 2025/0319070 A1
(43) Pub. Date: Oct. 16, 2025

(54) LOSARTAN LIQUID FORMULATIONS AND METHODS OF USE

(71) Applicant: Scienture, LLC, Commack, NY (US)

(72) Inventors: Bhavya Teja KOLLA, South Setauket, NY (US); Rahul SURANA, Commack, NY (US); Suketu SANGHVI, Kendall Park, NJ (US); Jigar BHATT, Stony Brook, NY (US)

(21) Appl. No.: 19/080,602

(22) Filed: Mar. 14, 2025

Related U.S. Application Data
(63) Continuation-in-part of application No. 18/061,819, filed on Dec. 5, 2022.
Application No. 18/061,819 is a division of application No. 18/052,116, filed on Nov. 2, 2022, now Pat. No. 11,890,273.
Application No. 18/052,116 is a continuation of application No. PCT/US2021/054054, filed on Oct. 7, 2021.
(60) Provisional application No. 63/089,950, filed on Oct. 9, 2020.

(51) Int. Cl. A61K 31/4178; A61K 9/00; A61K 9/08; A61K 9/10; A61K 9/107; A61K 47/14; A61K 47/32; A61K 47/34; A61K 47/36; A61K 47/38

(52) U.S. Cl. CPC A61K 31/4178; A61K 9/0053; A61K 9/08; A61K 9/10; A61K 9/107; A61K 47/14; A61K 47/32; A61K 47/34; A61K 47/36; A61K 47/38

(57) ABSTRACT

The present disclosure relates to stable, liquid pharmaceutical compositions of losartan or pharmaceutically acceptable salts thereof for oral administration. The present disclosure further provides powder compositions for reconstitution to provide a liquid formulation. In further aspects, the present disclosure relates to processes for preparation of such pharmaceutical compositions, and methods of treating a subject in need of losartan by administration of a formulation described herein.

### CROSS-REFERENCE TO RELATED PATENT APPLICATIONS

This application is a continuation-in-part of U.S. patent application Ser. No. 18/061,819, filed on Dec. 5, 2022, which is a divisional of U.S. patent application Ser. No. 18/052,116, filed on Nov. 2, 2022, now U.S. Pat. No. 11,890,273 issued Feb. 6, 2024, which is a continuation of International Application No. PCT/US2021/054054, filed on Oct. 7, 2021, which claims the benefit of U.S. Provisional Patent Application No. 63/089,950, filed on Oct. 9, 2020, each of which are incorporated herein by reference in their entirety.

### TECHNICAL FIELD

The present disclosure relates to stable, liquid pharmaceutical compositions of losartan or pharmaceutically acceptable salts thereof for oral administration. The present disclosure further provides powder compositions for reconstitution to provide a liquid formulation. In further aspects, the present disclosure relates to processes for preparation of such pharmaceutical compositions, and methods of treating a subject in need of losartan by administration of a formulation described herein.

### BACKGROUND

Hypertension is a serious medical condition wherein the blood pressure of a subject is elevated, increasing the risk of damage to various tissues and organs, including the heart, brain, and kidneys. According to a World Health Organization estimate, as of 2019, 1.13 billion people worldwide experience hypertension.

Losartan is an angiotensin II receptor blocker marketed under the trade name COZAAR®, which is indicated: (1) to lower blood pressure in adults and children greater than 6 years old; (2) to reduce the risk of stroke in patients with hypertension and left ventricular hypertrophy; and (3) to treat diabetic nephropathy with an elevated serum creatinine and proteinuria in patients with type 2 diabetes and a history of hypertension.

COZAAR® is available as 25 mg, 50 mg and 100 mg film coated tablets. A major problem associated with the tablet dosage form is the difficulty in oral administration for patients with swallowing difficulties, e.g., geriatric patients, pediatric patients, patients with neurological disorders, and patients with oral and esophageal cancers. Additionally, patients with underlying disease conditions may experience lower bioavailability from solid dosage forms of losartan.

The prescribing information supplied with COZAAR® describes the preparation of a losartan suspension by dispersing COZAAR® tablets with a suspending agent and a sweetener. This is the only commercially available liquid losartan preparation. However, these compounded preparations can be stored only up to 4 weeks under refrigerated conditions. Additionally, compounded preparations may not be bioequivalent to COZAAR® tablets, which may affect the therapeutic efficacy of such preparations.

Therefore, there remains a need for a stable, liquid pharmaceutical composition of losartan that is safely administrable and bioequivalent to COZAAR® tablets.

### BRIEF SUMMARY

### Pharmaceutical Composition

In some aspects, the present disclosure provides a pharmaceutical composition comprising losartan or a pharmaceutically acceptable salt thereof and one or more pharmaceutically acceptable excipients, wherein the pharmaceutical composition is suitable for oral administration, liquid, substantially free of impurities, and stable for at least 12 months. In some aspects, the pharmaceutical composition is bioequivalent to COZAAR®. In some aspects, the pharmaceutical composition is not bioequivalent to COZAAR®.

In some aspects, the present disclosure provides a pharmaceutical composition comprising losartan or a pharmaceutically acceptable salt thereof and one or more pharmaceutically acceptable excipients, wherein the pharmaceutical composition is suitable for oral administration, liquid, bioequivalent to COZAAR®, substantially free of impurities, and stable for at least 12 months.

### Forms of Losartan

In some aspects, the pharmaceutical composition comprises losartan.

In some aspects, the pharmaceutical composition comprises a pharmaceutically acceptable salt of losartan. In some aspects, the pharmaceutical composition comprises losartan potassium.

### Excipients

In some aspects, the pharmaceutical composition comprises two or more pharmaceutically acceptable excipients selected from the group consisting of: a suspending agent, a pH modifying agent, an emulsifying agent, an antioxidant, a chelating agent, a preservative, an antifoaming agent, a solubilizer, a vehicle, a surfactant or wetting agent, a sweetener, a stabilizer, a flavoring agent, and a colorant.

In some aspects, the pharmaceutical composition comprises a crystallization inhibitor or a crystal growth inhibitor. In some aspects, the crystallization inhibitor or crystal growth inhibitor is a polymer. In some aspects, the polymer is polyvinylpyrrolidone. In some aspects, the polymer is polyvinylpyrrolidone K90. In some aspects, the polymer is polyvinylpyrrolidone K30. In some aspects, the polymer is hydroxypropyl methylcellulose. In some aspects, the polymer is polyvinyl acetate. In some aspects, the polymer is a cyclodextrin. In some aspects, the cyclodextrin is hydroxypropyl β-cyclodextrin.

In some aspects, the one or more excipients comprise a suspending agent. In some aspects, the suspending agent is selected from the group consisting of: hydroxyethylcellulose, methylcellulose, hydroxymethylcellulose, hydroxypropylmethylcellulose, microcrystalline cellulose, sodium carboxymethylcellulose, xanthan gum, acacia, an alginate, and guar gum. In some aspects, the suspending agent comprises a thickening agent.

In some aspects, the suspending agent is present in an amount of 0.1 to 15 wt/wt %.

In some aspects, the one or more excipients comprise a pH modifying agent. In some aspects, the pH modifying agent is selected from the group consisting of: citric acid, sodium citrate, acetic acid, sodium acetate, sodium hydroxide, sodium dihydrogen phosphate, and disodium hydrogen phosphate.

In some aspects, the one or more excipients comprise an emulsifying agent selected from the group consisting of: a polysorbate, cetostearyl alcohol, and cetyl alcohol.

In some aspects, the one or more excipients comprise an antioxidant. In some aspects, the antioxidant is selected from the group consisting of: butylated hydroxyanisole, butylated hydroxytoluene, ascorbic acid, tocopherol, and vitamin E.

In some aspects, the pharmaceutical composition comprises an antioxidant in an amount of less than 10 wt/wt %.

In some aspects, the one or more excipients comprise a chelating agent. In some aspects, the chelating agent is ethylenediaminetetraacetic acid. In some aspects, the chelating agent is a disodium salt of ethylenediaminetetraacetic acid.

In some aspects, the one or more excipients comprise a preservative. In some aspects, the preservative is selected from the group consisting of: methyl paraben, ethyl paraben, propyl paraben, butyl paraben, benzoic acid, sodium benzoate, and benzalkonium chloride.

In some aspects, the pharmaceutical composition comprises a preservative in an amount of less than 10 wt/wt %.

In some aspects, the one or more excipients comprise an antifoaming agent. In some aspects, the pharmaceutical composition comprises an antifoaming agent in an amount of less than 10 wt/wt %.

In some aspects, the one or more excipients comprise a solubilizer. In some aspects, the solubilizer is cremophor. In some aspects, the solubilizer is vitamin E. In some aspects, the solubilizer is polyethylene glycol. In some aspects, the solubilizer is propylene glycol. In some aspects, the solubilizer comprises a co-solvent.

In some aspects, the one or more excipients comprise a vehicle. In some aspects, the vehicle is selected from the group consisting of: water, ethanol, glycerol, propylene glycol, polyethylene glycol, and an oil.

In some aspects, the one or more excipients comprise a surfactant or a wetting agent. In some aspects, the surfactant or wetting agent is selected from the group consisting of: a polysorbate, a polyoxamer, and sodium lauryl sulfate.

In some aspects, the one or more excipients comprise a sweetener. In some aspects, the sweetener is selected from the list consisting of: sorbitol, mannitol, xylitol, aspartame, sucralose, saccharine, and acesulfame K.

In some aspects, the pharmaceutical composition comprises a sweetener in an amount of less than 10 wt/wt %.

In some aspects, the one or more excipients comprise a stabilizer.

In some aspects, the one or more excipients comprise a flavoring agent. In some aspects, the flavoring agent is selected from the list consisting of: an apple flavoring agent, an orange flavoring agent, a mint flavoring agent, a cherry flavoring agent, and a strawberry flavoring agent.

In some aspects, the pharmaceutical composition comprises a flavoring agent in an amount of less than 10 wt/wt %.

In some aspects, the one or more excipients comprise a colorant.

Free from Impurities

In some aspects, the pharmaceutical composition comprises less than 5 wt/wt % of all impurities relative to the mass of losartan.

In some aspects, the pharmaceutical composition comprises less than 5 wt/wt % of an impurity relative to the mass of losartan.

In some aspects, the impurity is a product of losartan oxidation.

In some aspects, the impurity is a product of losartan degradation.

In some aspects, the impurity is a side product of a method of losartan synthesis.

In some aspects, the impurity is a byproduct of a method of losartan synthesis.

In some aspects, the impurity is losartan carboxylic acid.

In some aspects, the impurity is losartan impurity D. In some aspects, the pharmaceutical composition comprises less than 5 wt/wt % of losartan impurity D relative to the mass of losartan. In some aspects, the pharmaceutical composition comprises about 0.01 to 5 wt/wt % of losartan impurity D relative to the mass of losartan. In some aspects, the pharmaceutical composition comprises about 0.1 to 5 wt/wt % of losartan impurity D relative to the mass of losartan.

In some aspects, the impurity is losartan impurity E. In some aspects, the pharmaceutical composition comprises less than 5 wt/wt % of losartan impurity E relative to the mass of losartan. In some aspects, the pharmaceutical composition comprises about 0.01 to 5 wt/wt % of losartan impurity E relative to the mass of losartan. In some aspects, the pharmaceutical composition comprises about 0.1 to 5 wt/wt % of losartan impurity E relative to the mass of losartan.

In some aspects, the impurity is a biological contaminant. In some aspects, the impurity is a living biological contaminant. In some aspects, the impurity is a dead biological contaminant. In some aspects, the impurity is a viral contaminant. In some aspects, the impurity is a fungal contaminant. In some aspects, the impurity is a bacterial contaminant. In some aspects, the impurity is a bacterial endotoxin.

In some aspects, the biological contaminant is a replication-competent, metabolically inactive or minimally active biological product. In some aspects, the replication-competent, metabolically inactive or minimally active biological product is a spore.

In some aspects, the impurity is pyrogenic.

In some aspects, the impurity is detectable as visible particulate matter.

### Sterility

In some aspects, the pharmaceutical composition has been subjected to a sterilizing treatment. In some aspects, one or more components of the pharmaceutical composition have been subjected to a sterilizing treatment.

In some aspects, the sterilizing treatment comprises filtration. In some aspects, the sterilizing treatment comprises exposure to a high temperature. In some aspects, the sterilizing treatment comprises exposure to a low temperature. In some aspects, the sterilizing treatment comprises application of ultraviolet radiation. In some aspects, the sterilizing treatment is sufficient to kill a living biological contaminant.

### Stability

In some aspects, the pharmaceutically acceptable composition is stable as measured by one or more substantially unchanged characteristics after storage, the one or more substantially unchanged characteristics selected from the group consisting of: the concentration of losartan in the pharmaceutical composition; the concentration of an impurity in the pharmaceutical composition; the visual appearance of the pharmaceutical composition; the viscosity of the pharmaceutical composition; the uniformity of the pharmaceutical composition; and the sedimentation rate of the pharmaceutical composition.

In some aspects, the pharmaceutical composition is stable at 2° C.-8° C. and ≤65% relative humidity. In some aspects, the pharmaceutical composition is stable for 3 months at 2° C.-8° C. and ≤65% relative humidity. In some aspects, the pharmaceutical composition is stable for 4 months at 2° C.-8° C. and ≤65% relative humidity. In some aspects, the pharmaceutical composition is stable for 6 months at 2° C.-8° C. and ≤65% relative humidity. In some aspects, the pharmaceutical composition is stable for 8 months at 2° C.-8° C. and ≤65% relative humidity. In some aspects, the pharmaceutical composition is stable for 10 months at 2° C.-8° C. and ≤65% relative humidity. In some aspects, the pharmaceutical composition is stable for 12 months at 2° C.-8° C. and ≤65% relative humidity. In some aspects, the pharmaceutical composition is stable for 16 months at 2° C.-8° C. and ≤65% relative humidity. In some aspects, the pharmaceutical composition is stable for 18 months at 2° C.-8° C. and ≤65% relative humidity. In some aspects, the pharmaceutical composition is stable for 24 months at 2° C.-8° C. and ≤65% relative humidity.

In some aspects, the pharmaceutical composition is stable at 15° C.-25° C. and ≤65% relative humidity. In some aspects, the pharmaceutical composition is stable for 3 months at 15° C.-25° C. and ≤65% relative humidity. In some aspects, the pharmaceutical composition is stable for 4 months at 15° C.-25° C. and ≤65% relative humidity. In some aspects, the pharmaceutical composition is stable for 6 months at 15° C.-25° C. and ≤65% relative humidity. In some aspects, the pharmaceutical composition is stable for 8 months at 15° C.-25° C. and ≤65% relative humidity. In some aspects, the pharmaceutical composition is stable for 10 months at 15° C.-25° C. and ≤65% relative humidity. In some aspects, the pharmaceutical composition is stable for 12 months at 15° C.-25° C. and ≤65% relative humidity. In some aspects, the pharmaceutical composition is stable for 16 months at 15° C.-25° C. and ≤65% relative humidity. In some aspects, the pharmaceutical composition is stable for 18 months at 15° C.-25° C. and ≤65% relative humidity. In some aspects, the pharmaceutical composition is stable for 24 months at 15° C.-25° C. and ≤65% relative humidity.

In some aspects, the pharmaceutical composition is stable at 30° C. and ≤65% relative humidity. In some aspects, the pharmaceutical composition is stable for 3 months at 30° C. and ≤65% relative humidity. In some aspects, the pharmaceutical composition is stable for 4 months at 30° C. and ≤65% relative humidity. In some aspects, the pharmaceutical composition is stable for 6 months at 30° C. and ≤65% relative humidity. In some aspects, the pharmaceutical composition is stable for 8 months at 30° C. and ≤65% relative humidity. In some aspects, the pharmaceutical composition is stable for 10 months at 30° C. and ≤65% relative humidity. In some aspects, the pharmaceutical composition is stable for 12 months at 30° C. and ≤65% relative humidity. In some aspects, the pharmaceutical composition is stable for 16 months at 30° C. and ≤65% relative humidity. In some aspects, the pharmaceutical composition is stable for 18 months at 30° C. and ≤65% relative humidity. In some aspects, the pharmaceutical composition is stable for 24 months at 30° C. and ≤65% relative humidity.

In some aspects, the pharmaceutical composition is stable at 40° C. and 75% relative humidity. In some aspects, the pharmaceutical composition is stable for 3 months at 40° C. and 75% relative humidity. In some aspects, the pharmaceutical composition is stable for 4 months at 40° C. and 75% relative humidity. In some aspects, the pharmaceutical composition is stable for 6 months at 40° C. and 75% relative humidity. In some aspects, the pharmaceutical composition is stable for 8 months at 40° C. and 75% relative humidity. In some aspects, the pharmaceutical composition is stable for 10 months at 40° C. and 75% relative humidity. In some aspects, the pharmaceutical composition is stable for 12 months at 40° C. and 75% relative humidity. In some aspects, the pharmaceutical composition is stable for 16 months at 40° C. and 75% relative humidity. In some aspects, the pharmaceutical composition is stable for 18 months at 40° C. and 75% relative humidity. In some aspects, the pharmaceutical composition is stable for 24 months at 40° C. and 75% relative humidity.

### Liquid and Powder Forms

In some aspects, the pharmaceutical composition of the present disclosure is a solution, an emulsion, or a suspension. In some aspects, the present disclosure provides for a concentrated form of losartan or a pharmaceutically acceptable salt thereof, which can be combined with a suitable diluent to prepare a pharmaceutical composition described herein. In some aspects, the concentrated form of losartan or a pharmaceutically acceptable salt thereof is selected from the group consisting of: a powder, a plurality of granules, modified losartan, and a concentrated liquid form (e.g., a concentrated solution or a low-volume mixed solid and liquid phase). In some aspects, the concentrated form of losartan or a pharmaceutically acceptable salt thereof can be combined with a suitable diluent to prepare one or more of a solution, a suspension, and an emulsion.

In some aspects, the concentrated form of losartan or a pharmaceutically acceptable salt thereof is combined with the suitable diluent and heated to form a pharmaceutically acceptable composition disclosed herein. In some aspects, the concentrated form of losartan or a pharmaceutically acceptable salt thereof is combined with the suitable diluent and an additional component to form a pharmaceutically acceptable composition disclosed herein. In some aspects, the additional component is a pH modifying agent. In some aspects, the pH modifying agent is an acid. In some aspects, the pH modifying agent is a base.

### Solution

In some aspects, the pharmaceutical composition is a solution. In some aspects, the pharmaceutical composition is a solution and the pharmaceutical composition comprises losartan. In some aspects, the pharmaceutical composition is a solution and the pharmaceutical composition comprises a pharmaceutically acceptable salt of losartan. In some aspects, the pharmaceutical composition is a solution and the pharmaceutical composition comprises losartan potassium.

In some aspects, the pharmaceutical composition is a solution comprising losartan substantially dissolved in a pharmaceutically acceptable vehicle. In some aspects, the solution comprises at least 75% of losartan or pharmaceutically acceptable salt thereof in dissolved form. In some aspects, the solution comprises at least 90% of losartan or pharmaceutically acceptable salt thereof in dissolved form.

In some aspects, the solution further comprises one or more inactive ingredients selected from the group consisting of: co-solvents, pH modifying agents, surfactants, antioxidants, preservatives, and flavoring agents.

### Emulsion

In some aspects, the pharmaceutical composition is an emulsion. In some aspects, the pharmaceutical composition is an emulsion and the pharmaceutical composition comprises losartan. In some aspects, the pharmaceutical composition is an emulsion and the pharmaceutical composition comprises a pharmaceutically acceptable salt of losartan. In some aspects, the pharmaceutical composition is an emulsion and the pharmaceutical composition comprises losartan potassium.

In some aspects, the emulsion is a multiple emulsion. In some aspect, the emulsion is a microemulsion.

In some aspects, the emulsion comprises at least two immiscible liquid phases wherein one liquid phase is in the form of globules. In some aspects, the pharmaceutical composition comprises an emulsifying agent. In some aspects, the pharmaceutical composition is an emulsion wherein a hydrophobic phase comprises at least 50% of losartan or a pharmaceutically acceptable salt thereof. In some aspects, the pharmaceutical composition is an emulsion wherein a hydrophobic phase comprises at least 60% of losartan or a pharmaceutically acceptable salt thereof. In some aspects, the pharmaceutical composition is an emulsion wherein a hydrophobic phase comprises at least 70% of losartan or a pharmaceutically acceptable salt thereof. In some aspects, the pharmaceutical composition is an emulsion wherein a hydrophobic phase comprises at least 80% of losartan or a pharmaceutically acceptable salt thereof. In some aspects, the pharmaceutical composition is an emulsion wherein a hydrophobic phase comprises at least 90% of losartan or a pharmaceutically acceptable salt thereof.

### Suspension

In some aspects, the pharmaceutical composition is a suspension. In some aspects, the pharmaceutical composition is a suspension and the pharmaceutical composition comprises losartan. In some aspects, the pharmaceutical composition is a suspension and the pharmaceutical composition comprises a pharmaceutically acceptable salt of losartan. In some aspects, the pharmaceutical composition is a suspension and the pharmaceutical composition comprises losartan potassium.

In some aspects, the suspension is a cloudy biphasic liquid comprising a plurality of losartan particles uniformly dispersed in a pharmaceutically acceptable vehicle. In some aspects, at least 50% of losartan or pharmaceutically acceptable salt thereof is in suspended form. In some aspects, at least 60% of losartan or pharmaceutically acceptable salt thereof is in suspended form. In some aspects, at least 70% of losartan or pharmaceutically acceptable salt thereof is in suspended form. In some aspects, at least 80% of losartan or pharmaceutically acceptable salt thereof is in suspended form. In some aspects, at least 90% of losartan or pharmaceutically acceptable salt thereof is in suspended form.

In some aspects, the suspension comprises a flocculating agent.

### Powder

In some aspects, the present disclosure provides a powder which is combined with a liquid to produce the pharmaceutical composition described herein.

In some aspects, the powder comprises losartan or a pharmaceutically acceptable salt thereof. In some aspects, the powder comprises losartan. In some aspects, the powder comprises losartan potassium.

In some aspects, the powder comprises one or more pharmaceutically acceptable excipients.

In some aspects, the losartan powder is bioequivalent to COZAAR®.

In some aspects, the powder is sterile.

In some aspects, the powder is non-pyrogenic.

In some aspects, the powder is substantially free of impurities.

In some aspects, the liquid is a solvent. In some aspects, the solvent is water.

In some aspects, the liquid is provided in a container. In some aspects, the container is a bottle. In some aspects, the bottle is an amber bottle.

In some aspects, the container contains a suspending agent. In some aspects, the container contains a preservative. In some aspects, the container contains a sweetener. In some aspects, the container contains a flavoring agent.

In some aspects, the powder is packaged in a sachet.

### Granules

In some aspects, the present disclosure provides a plurality of granules which is combined with a liquid to produce the pharmaceutical composition described herein.

In some aspects, the plurality of granules comprises losartan or a pharmaceutically acceptable salt thereof. In some aspects, the plurality of granules comprises losartan. In some aspects, the plurality of granules comprises losartan potassium.

In some aspects, the plurality of granules comprises one or more pharmaceutically acceptable excipients.

In some aspects, the plurality of granules is bioequivalent to COZAAR®.

In some aspects, the plurality of granules is sterile.

In some aspects, the plurality of granules is non-pyrogenic.

In some aspects, the plurality of granules is substantially free of impurities.

In some aspects, the liquid is a solvent. In some aspects, the solvent is water.

In some aspects, the liquid is provided in a container. In some aspects, the container is a bottle. In some aspects, the bottle is an amber bottle.

In some aspects, the container contains a suspending agent. In some aspects, the container contains a preservative. In some aspects, the container contains a sweetener. In some aspects, the container contains a flavoring agent.

In some aspects, the plurality of granules is packaged in a sachet.

### Particle Characterization

In some aspects, the suspension or the powder exhibits a dissolution profile as measured using a paddle type apparatus. In some aspects, the dissolution profile is measured at 50 rpm. In some aspects, the dissolution profile of the suspension or powder is measured in a mixture of water and 0.1N HCl. In some aspects, less than 35% of the plurality of losartan particles are dissolved in 15 minutes. In some aspects, less than 70% of the plurality of losartan particles are dissolved in 30 minutes. In some aspects, at least 80% of the plurality of losartan particles are dissolved in 60 minutes. In some aspects, at least 80% of the plurality of losartan particles are dissolved in 15 minutes. In some aspects, at least 85% of the plurality of losartan particles are dissolved in 15 minutes. In some aspects, at least 90% of the plurality of losartan particles are dissolved in 15 minutes. In some aspects, at least 95% of the plurality of losartan particles are dissolved in 15 minutes. In some aspects, about 100% of the plurality of losartan particles are dissolved in 15 minutes. In some aspects, at least 80% of the plurality of losartan particles are dissolved in 5 minutes. In some aspects, at least 85% of the plurality of losartan particles are dissolved in 5 minutes. In some aspects, at least 90% of the plurality of losartan particles are dissolved in 5 minutes. In some aspects, at least 95% of the plurality of losartan particles are dissolved in 5 minutes. In some aspects, about 100% of the plurality of losartan particles are dissolved in 5 minutes.

In some aspects, the suspension or powder exhibits a particle size distribution as measured using a particle size analyzer. In some aspects, the particle size analyzer is a Malvern Mastersizer particle size analyzer. In some aspects, the particle size analyzer is a Malvern Mastersizer 3000 particle size analyzer. In some aspects, the particle size analyzer is a Malvern Zetasizer particle size analyzer.

In some aspects, the particle size distribution comprises a D10 value less than 100 μm. In some aspects, the particle size distribution comprises a D50 value less than 500 μm. In some aspects, the particle size distribution comprises a D90 value less than 1000 μm. In some aspects, the particle size distribution comprises a D10 value less than 100 μm, a D50 value less than 500 μm, and a D90 value less than 1000 μm.

In some aspects, the particle size distribution comprises a D10 value less than 20 μm. In some aspects, the particle size distribution comprises a D50 value less than 100 μm. In some aspects, the particle size distribution comprises a D90 value less than 300 μm. In some aspects, the particle size distribution comprises a D10 value less than 20 μm, a D50 value less than 100 μm, and a D90 value less than 300 μm.

In some aspects, the particle size distribution comprises a D10 value less than 2 μm. In some aspects, the particle size distribution comprises a D50 value less than 10 μm. In some aspects, the particle size distribution comprises a D90 value less than 30 μm. In some aspects, the particle size distribution comprises a D10 value less than 2 μm, a D50 value less than 10 μm, and a D90 value less than 30 μm.

In some aspects, the particle size distribution comprises a D10 value less than 100 nm. In some aspects, the particle size distribution comprises a D50 value less than 500 nm. In some aspects, the particle size distribution comprises a D90 value less than 1000 nm. In some aspects, the particle size distribution comprises a D10 value less than 100 nm, a D50 value less than 500 nm, and a D90 value less than 1000 nm.

In some aspects, the particle size distribution comprises a D90 value less than 2 μm. In some aspects, the particle size distribution comprises a D90 value less than 1 μm. In some aspects, the particle size distribution comprises a D90 value less than 500 nm.

In some aspects, the particle size distribution comprises a D10 value less than or equal to 1 μm. In some aspects, the particle size distribution comprises a D50 value less than or equal to 5 μm. In some aspects, the particle size distribution comprises a D90 value less than or equal to 15 μm. In some aspects, the particle size distribution comprises a D10 value less than or equal to 1 μm, a D50 value less than or equal to 5 μm, and a D90 value less than or equal to 15 μm.

In some aspects, the suspension or the powder has a sedimentation rate of less than 10% over a period of 24 hours. In some aspects, the suspension or the powder has a sedimentation rate of less than 5% over a period of 24 hours.

### Modified Losartan

In some aspects, the pharmaceutical composition comprises a modified losartan. In some aspects, the powder comprises a modified losartan. In some aspects, the modified losartan comprises losartan or a pharmaceutically acceptable salt thereof complexed or coated with a wax, one or more polymers, or one or more other inactive ingredients. In some aspects, the modified losartan comprises losartan or a pharmaceutically acceptable salt thereof complexed or coated with a wax. In some aspects, the modified losartan comprises losartan or a pharmaceutically acceptable salt thereof complexed or coated with one or more polymers. In some aspects, the modified losartan is losartan or a pharmaceutically acceptable salt thereof coated with one or more inactive ingredients.

### Crystalline Form

In some aspects, the powder or suspension comprises losartan or a pharmaceutically acceptable salt thereof in crystalline form. In some aspects, the crystalline form is thermodynamically stable. In some aspects, the crystalline form is thermodynamically stable, as measured by a substantially unchanged X-ray powder diffraction (XRPD) profile following storage. In some aspects, the crystalline form is thermodynamically stable, as measured by a substantially unchanged differential scanning calorimetry (DSC) profile following storage.

### Concentration of Losartan

In some aspects, the pharmaceutical composition comprises about 1 mg/mL to about 50 mg/mL of losartan or pharmaceutically acceptable salt thereof. In some aspects, the pharmaceutical composition comprises about 2 mg/mL to about 20 mg/mL of losartan or a pharmaceutically acceptable salt thereof. In some aspects, the pharmaceutical composition comprises about 5 mg/mL to about 20 mg/mL of losartan or a pharmaceutically acceptable salt thereof. In some aspects, the pharmaceutical composition comprises about 8 mg/mL to about 12 mg/mL of losartan or a pharmaceutically acceptable salt thereof.

pH

In some aspects, the pharmaceutical composition has a pH between 2 and 10. In some aspects, the pharmaceutical composition has a pH between 2 and 7. In some aspects, the pharmaceutical composition has a pH of about 6. In some aspects, the pharmaceutical composition has a pH of about 4. In some aspects, the pharmaceutical composition has a pH of 4.2. In some aspects, the pharmaceutical composition has a pH of about 5. In some aspects, the pharmaceutical composition has a pH less than 6. In some aspects, the pharmaceutical composition has a pH greater than 6. In some aspects, the pharmaceutical composition has a pH between 7 and 9. In some aspects, the pharmaceutical composition has a pH between 3 and 5. In some aspects, the pharmaceutical composition has a pH between 5 and 7.

### Pharmacokinetic Parameters and Bioequivalence

In some aspects, the mean Tₘₐₓ of the pharmaceutical composition described herein is about 98% to about 102% of the mean Tₘₐₓ of COZAAR®. In some aspects, the mean Tₘₐₓ of the pharmaceutical composition described herein is about 95% to about 105% of the mean Tₘₐₓ of COZAAR®. In some aspects, the mean Tₘₐₓ of the pharmaceutical composition described herein is about 90% to about 110% of the mean Tₘₐₓ of COZAAR®. In some aspects, the mean Tₘₐₓ of the pharmaceutical composition described herein is about 80% to about 125% of the mean Tₘₐₓ of COZAAR®. In some aspects, the mean Tₘₐₓ of the pharmaceutical composition described herein is about 75% to about 130% of the mean Tₘₐₓ of COZAAR®. In some aspects, the mean Tₘₐₓ of the pharmaceutical composition described herein is about 70% to about 135% of the mean Tₘₐₓ of COZAAR®. In some aspects, the mean Tₘₐₓ of the pharmaceutical composition described herein is about 65% to about 140% of the mean Tₘₐₓ of COZAAR®. In some aspects, the mean Tₘₐₓ of the pharmaceutical composition described herein is about 60% to about 145% of the mean Tₘₐₓ of COZAAR®. In some aspects, the mean Tₘₐₓ of the pharmaceutical composition described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the mean Tₘₐₓ of COZAAR®.

In some aspects, the mean Tₘₐₓ of the pharmaceutical composition described herein is about 50% to about 150% of the mean Tₘₐₓ of COZAAR®. In some aspects, the mean Tₘₐₓ of the pharmaceutical composition described herein is about 40% to about 160% of the mean Tₘₐₓ of COZAAR®. In some aspects, the mean Tₘₐₓ of the pharmaceutical composition described herein is about 30% to about 170% of the mean Tₘₐₓ of COZAAR®. In some aspects, the mean Tₘₐₓ of the pharmaceutical composition described herein is about 20% to about 200% of the mean Tₘₐₓ of COZAAR®.

In some aspects, the mean Cₘₐₓ of the pharmaceutical composition described herein is about 98% to about 102% of the mean Cₘₐₓ of COZAAR®. In some aspects, the mean Cₘₐₓ of the pharmaceutical composition described herein is about 95% to about 105% of the mean Cₘₐₓ of COZAAR®. In some aspects, the mean Cₘₐₓ of the pharmaceutical composition described herein is about 90% to about 110% of the mean Cₘₐₓ of COZAAR®. In some aspects, the mean Cₘₐₓ of the pharmaceutical composition described herein is about 80% to about 125% of the mean Cₘₐₓ of COZAAR®. In some aspects, the mean Cₘₐₓ of the pharmaceutical composition described herein is about 75% to about 130% of the mean Cₘₐₓ of COZAAR®. In some aspects, the mean Cₘₐₓ of the pharmaceutical composition described herein is about 70% to about 135% of the mean Cₘₐₓ of COZAAR®. In some aspects, the mean Cₘₐₓ of the pharmaceutical composition described herein is about 65% to about 140% of the mean Cₘₐₓ of COZAAR®. In some aspects, the mean Cₘₐₓ of the pharmaceutical composition described herein is about 60% to about 145% of the mean Cₘₐₓ of COZAAR®. In some aspects, the mean Cₘₐₓ of the pharmaceutical composition described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the mean Cₘₐₓ of COZAAR®.

In some aspects, the mean AUC_{0-∞} of the pharmaceutical composition described herein is about 98% to about 102% of the mean AUC_{0-∞} of COZAAR®. In some aspects, the mean AUC_{0-∞} of the pharmaceutical composition described herein is about 95% to about 105% of the mean AUC_{0-∞} of COZAAR®. In some aspects, the mean AUC_{0-∞} of the pharmaceutical composition described herein is about 90% to about 110% of the mean AUC0-% of COZAAR®. In some aspects, the mean AUC_{0-∞} of the pharmaceutical composition described herein is about 80% to about 125% of the mean AUC0-% of COZAAR®. In some aspects, the mean AUC_{0-∞} of the pharmaceutical composition described herein is about 75% to about 130% of the mean AUC_{0-∞} of COZAAR®. In some aspects, the mean AUC_{0-∞} of the pharmaceutical composition described herein is about 70% to about 135% of the mean AUC_{0-∞} of COZAAR®. In some aspects, the mean AUC_{0-∞} of the pharmaceutical composition described herein is about 65% to about 140% of the mean AUC_{0-∞} of COZAAR®. In some aspects, the mean AUC_{0-∞} of the pharmaceutical composition described herein is about 60% to about 145% of the mean AUC_{0-∞} of COZAAR®. In some aspects, the mean AUC_{0-∞} of the pharmaceutical composition described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the mean AUC0-0 of COZAAR®.

### Additional Active Ingredients

In some aspects, the pharmaceutical composition comprises one or more active ingredients in addition to losartan or a pharmaceutically acceptable salt thereof. In some aspects, the one or more active ingredients are selected from the group consisting of: calcium channel blockers, diuretics, ACE inhibitors, and beta blockers. In some aspects, the one or more active ingredients are calcium channel blockers. In some aspects, the one or more active ingredients are diuretics. In some aspects, the one or more active ingredients are ACE inhibitors. In some aspects, the one or more active ingredients are beta blockers.

### Viscosity

In some aspects, the pharmaceutical composition has a viscosity of less than 2000 centipoise, as measured using a Brookfield viscometer. In some aspects, the pharmaceutical composition has a viscosity of less than 1000 centipoise, as measured using a Brookfield viscometer.

### Volume

In some aspects, the pharmaceutical composition is administered in a volume of 0.5 mL to 50 mL. In some aspects, the pharmaceutical composition is administered in a volume of less than 100 mL. In some aspects, the pharmaceutical composition is administered in a volume of about 2.5 mL, about 5 mL, or about 10 mL.

### Container

In some aspects, a pharmaceutical composition is packaged in a glass container. In some aspects, the powder is packaged in a glass container. In some aspects, the glass container is an amber glass container. In some aspects, the amber glass container is child resistant. In some aspects, the amber glass container comprises a child resistant cap. In some aspects, the pharmaceutical composition is packaged in a polymeric container. In some aspects, the polymeric container is child resistant. In some aspects, the polymeric container comprises a child resistant cap. In some aspects, the polymeric container comprises high density polyethylene (HDPE). In some aspects, the polymeric container comprises low density polyethylene (LDPE).

### Kit

In some aspects, the present disclosure provides for a kit comprising a pharmaceutically acceptable composition described herein, and further comprising a set of instructions for administration of the pharmaceutically acceptable composition to a subject in need thereof. In some aspects, the set of instructions comprises an instruction to add an amount of a diluent to a container comprising a concentrated form of losartan or a pharmaceutically acceptable salt thereof, wherein the kit comprises the container. In some aspects, the diluent is water.

In some aspects, the present disclosure provides for a kit comprising: a concentrated form of losartan or a pharmaceutically acceptable salt thereof; a diluent; and a set of instructions, wherein the set of instructions comprises: instructions for combining the concentrated form of losartan or a pharmaceutically acceptable salt thereof and the diluent to form a pharmaceutically acceptable composition described herein; and instructions for administration of the pharmaceutically acceptable composition to a subject in need thereof. In some aspects, the concentrated form of losartan or a pharmaceutically acceptable salt thereof is in the form of a powder or a plurality of granules.

In some aspects, the kit comprises a concentrated form of a pharmaceutical composition disclosed herein. In some aspects, the concentrated form is prepared by combining components of a pharmaceutical composition disclosed herein without dilution to a final concentration suitable for administration to a subject. In some aspects, the concentrated form is a concentrated liquid form. In some aspects, the concentrated liquid form is a solution. In some aspects, the concentrated liquid form is a suspension. In some aspects, the concentrated form is a dry form. In some aspects, the dry form comprises less than 0.1%, 0.5%, 1%, 2%, 3%, 4%, 5%, 6%, 7%, 8%, 9%, or 10% of water by weight of the dry form. In some aspects, the dry form is a powder or a plurality of granules.

### Method of Preparing Suspension

In some aspects, the present disclosure provides a method of preparing a suspension described herein, the method comprising combining a plurality of losartan particles and a suspending agent. In some aspects, the plurality of losartan particles is combined with the suspending agent.

In some aspects, the present disclosure provides a method of preparing a suspension described herein, the method comprising dissolving losartan or a pharmaceutically acceptable salt thereof in a suitable fluid (e.g., water), adjusting the pH of the suitable liquid comprising losartan or a pharmaceutically acceptable salt thereof, and combining the suitable liquid comprising losartan or a pharmaceutically acceptable salt thereof with the remaining components of the suspension, thereby forming the suspension.

In some aspects, the present disclosure provides a method of preparing a suspension described herein, the method comprising combining losartan or a pharmaceutically acceptable salt thereof and one or more excipients, granulating the composition with a suitable fluid (e.g., water), drying the resulting composition to form a dry composition, and combining the dry composition with a suitable liquid, thereby forming the suspension.

In some aspects, the present disclosure provides a method of preparing a suspension described herein, the method comprising a step of particle size reduction. In some aspects, the step of particle size reduction comprises wet milling. In some aspects, the step of particle size reduction comprises microfluidization. In some aspects, the step of particle size reduction comprises homogenization. In some aspects, the step of particle size reduction comprises nano-milling.

In some aspects, a pH modifying agent is added after the plurality of losartan particles is combined with the suspending agent. In some aspects, the pH modifying agent is selected from the group consisting of: citric acid, sodium citrate, acetic acid, sodium acetate, sodium hydroxide, dibasic calcium phosphate, sodium dihydrogen phosphate, and disodium hydrogen phosphate. In some aspects, the pH modifying agent is a buffer.

In some aspects, the one or more polymers are sprayed by dissolving the one or more polymers in a volatile solvent. In some aspects, the volatile solvent is selected from the group consisting of: ethanol, acetone, or isopropyl alcohol. In some aspects, the volatile solvent is ethanol. In some aspects, the volatile solvent is acetone. In some aspects, the volatile solvent is isopropyl alcohol.

In some aspects, heated air is added to cause evaporation of the volatile solvent. In some aspects, evaporation of the volatile solvent causes deposition of the one or more polymers onto the surface of a particle comprising losartan or a pharmaceutically acceptable salt thereof. In some aspects, the particles comprising losartan or a pharmaceutically acceptable salt thereof are dried to form a modified losartan. In some aspects, the plurality of losartan particles is provided as a modified losartan which is preformed. In some aspects, the plurality of losartan particles comprises losartan or a pharmaceutically acceptable salt thereof.

In some aspects, the plurality of losartan particles is fluidized in a fluidized bed system, and are then coated with one or more polymers. In some aspects, the plurality of losartan particles comprises losartan or a pharmaceutically acceptable salt thereof. In some aspects, the plurality of losartan particles has a particle size of less than 1000 μm. In some aspects, the plurality of losartan particles has a particle size of less than 750 μm. In some aspects, the plurality of losartan particles has a particle size of less than 500 μm. In some aspects, the plurality of losartan particles has a particle size of less than 250 μm.

In some aspects, the one or more polymers comprise pH dependent polymers (e.g., Eudragit L, Eudragit S, or cellulose acetate phthalate) and/or pH independent polymers (e.g., Eudragit RS, Eudragit RL, or cellulose acetate).

In some aspects, the modified losartan tastes less bitter than losartan.

In some aspects, the modified losartan is more stable than losartan.

In some aspects, a composition of the present disclosure is diluted prior to administration. In some aspects, the composition is diluted with a diluent. In some aspects, the diluent is a composition disclosed herein, but excludes losartan (i.e., contains the same components and concentrations as a composition disclosed herein, with the exception that losartan or a pharmaceutically acceptable salt thereof is not included in the diluent).

In some aspects, a diluted composition disclosed herein is for administration to a pediatric subject. In some aspects, a diluted composition disclosed herein is for administration to a neonatal subject. In some aspects, a diluted composition disclosed herein is for administration to a low weight subject.

### Method of Preparing Solution or Emulsion

In some aspects, the present disclosure provides a method of preparing a solution or an emulsion described herein, comprising combining the components of the solution or the emulsion.

In some aspects, losartan or a pharmaceutically acceptable salt thereof is dissolved in a hydrophobic phase (e.g., vegetable oil). In some aspects, an antioxidant is added to a hydrophobic phase. In some aspects, the hydrophobic phase comprising losartan or a pharmaceutically acceptable salt thereof is mixed with a hydrophilic phase. In some aspects, the hydrophilic phase comprises a sweetener. In some aspects, the hydrophilic phase comprises a flavoring agent. In some aspects, the hydrophilic phase comprises Polysorbate 80 and/or Tweens. In some aspects, the emulsion is prepared by combining and mixing the hydrophobic phase and the hydrophilic phase. In some aspects, the hydrophobic phase and hydrophilic phase are mixed at a high speed using a homogenizer.

### Method of Preparing Powder

In some aspects, the present disclosure provides a method of preparing a powder, the method comprising dry blending the components of a pharmaceutical composition described herein. In some aspects, the pharmaceutical composition is a suspension described herein.

### Method of Preparing Modified Losartan

In some aspects, the present disclosure provides a method of preparing modified losartan, the method comprising complexing or coating losartan or a pharmaceutically acceptable salt thereof with a substance, wherein the substance is a wax, a polymer, or one or more other inactive ingredients. In some aspects, the modified losartan is formed during preparation of the pharmaceutical composition. In some aspects, modified losartan is formed by complexation with an ion exchange resin. In some aspects, modified losartan is formed by complexation with a cyclodextrin. In some aspects, the modified losartan is prepared by microencapsulation of losartan or a pharmaceutically acceptable salt thereof in a coat of inactive ingredients.

### Method of Treatment

In some aspects, the present disclosure provides a method of treating a subject in need thereof comprising administering to the subject a therapeutically effective amount of the pharmaceutical composition described herein.

In some aspects, the subject has been diagnosed with pre-hypertension or hypertension. In some aspects, the subject has been diagnosed with pre-hypertension. In some aspects, the subject has been diagnosed with hypertension. In some aspects, the subject has a history of hypertension.

In some aspects, the subject has been diagnosed with left ventricular hypertrophy. In some aspects, the subject has been diagnosed with both hypertension and left ventricular hypertrophy.

In some aspects, the subject has been diagnosed with type 2 diabetes.

In some aspects, the subject has been diagnosed with nephropathy.

In some aspects, the subject has been diagnosed with diabetic nephropathy.

In some aspects, the subject has been diagnosed with proteinuria.

In some aspects, the subject exhibits a urinary albumin to creatinine ratio of greater than or equal to 300 mg/g.

In some aspects, the subject has been diagnosed with an elevated serum creatinine.

In some aspects, the pharmaceutical composition is co-administered with at least one other pharmaceutical agent. In some aspects, the at least one pharmaceutical agent is an antihypertensive agent. In some aspects, the antihypertensive agent is selected from the group consisting of: an angiotensin II antagonist, an angiotensin converting enzyme inhibitor, or a neutral endopeptidase/angiotensin converting enzyme inhibitor. In some aspects, the antihypertensive agent is an angiotensin II antagonist. In some aspects, the antihypertensive agent is an angiotensin converting enzyme inhibitor. In some aspects, the antihypertensive agent is a neutral endopeptidase/angiotensin converting enzyme inhibitor.

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “modified losartan” as used herein refers to losartan or a pharmaceutically acceptable salt thereof, wherein losartan or a pharmaceutically acceptable salt thereof is complexed or coated with inactive ingredients. In some aspects, a modified losartan is more stable than losartan or a pharmaceutically acceptable salt thereof not complexed or coated with inactive ingredients. In some aspects, a suspension comprising a modified losartan is more stable than a suspension comprising losartan or a pharmaceutically acceptable salt thereof not complexed or coated with inactive ingredients. In some aspects, a modified losartan is less soluble in water than losartan or a pharmaceutically acceptable salt thereof not complexed or coated with inactive ingredients. In some aspects, a modified losartan is more resistant to degradation or oxidation than losartan or a pharmaceutically acceptable salt thereof not complexed or coated with inactive ingredients.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes. A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21th edition, Gennaro, Ed., Lippincott Williams & Wilkins (2005).

The term “effective amount” or “pharmaceutically effective amount” or “therapeutically effective amount” as used herein refers to the amount or quantity of a drug (e.g., losartan or a pharmaceutically acceptable salt thereof) or pharmaceutically active substance which is sufficient to elicit the required or desired therapeutic response, or in other words, the amount which is sufficient to elicit an appreciable biological response when administered to a patient.

The term “unit dosage form” or “unit dose composition” as used herein refers to a device containing a quantity of the therapeutic compound, said quantity being such that one or more predetermined units may be provided as a single therapeutic administration.

The term “Cₘₐₓ” as used herein refers to the maximum plasma concentration of a drug after administration of the drug.

The term “Tₘₐₓ” as used herein refers to the time required to reach the maximal plasma concentration Cₘₐₓ after administration of a drug.

The term “AUC” as used herein refers to the area under the curve of a plot of plasma concentration versus time following administration of a drug.

The term “AUC0-1” as used herein refers to the area under the drug concentration-time curve from time zero to the time of the last measurable concentration (Ct).

The term “AUC₀₋₄” as used herein refers to the area under the drug concentration-time curve from time zero to infinity.

The term “steady state” as used herein means that the amount of the drug reaching the system is approximately the same as the amount of the drug leaving the system. Thus, at “steady-state,” the patient's body eliminates the drug at approximately the same rate that the drug becomes available to the patient's system through absorption into the blood stream.

The term “mean” refers to an average value in a patient population. For example, a “mean Cₘₐₓ” refers to an average of the maximum plasma concentrations of a drug in a patient population.

The term “treating” or “treatment” as used herein refers to the administration of a composition to a subject for therapeutic purposes.

The term “serum concentration” generally refers to the amount of a drug or other compound in the circulation, both bound to proteins and unbound, the latter of which generally corresponds to the therapeutically active fraction.

The term “bioavailability” generally refers to the rate and extent to which the active ingredient is absorbed from a drug product and becomes available at the site of action.

“Bioequivalence” is a term in pharmacokinetics generally used to assess the expected in vivo biological equivalence of two proprietary preparations of a drug. Two pharmaceutical products are bioequivalent if they are pharmaceutically equivalent and their bioavailabilities (rate and extent of availability) after administration in the same molar dose are similar to such a degree that their effects, with respect to both efficacy and safety, can be expected to be essentially the same. Bioequivalence criteria include The United States Food and Drug Administration (“FDA”) requirement for bioequivalence studies to have percentage ratios of the test vs reference for Cₘₐₓ and AUC, at 90% confidence interval, between 80% and 125%.

The term “stable” as used herein means a composition is substantially unchanged after storage for a period of time under given storage conditions. In some aspects, a composition is “stable” if it meets the International Council for Harmonisation of Technical Requirements for Pharmaceuticals for Human Use (ICH) requirements for stability. In some aspects, a composition is considered stable if after a period of at least 12 months of storage at 15° C.-25° C. and ≤65% relative humidity, the concentration or integrity of carrier components or losartan or a pharmaceutically acceptable salt thereof is substantially unchanged (e.g., a loss of no more than 1%, 2%, 3%, 4%, 5%, 6%, 7%, 8%, 9%, or 10% of losartan or a pharmaceutically acceptable salt thereof). Stability can also be analyzed by subjecting composition to accelerated stability conditions for a shorter period of time (e.g., 6 months at 30° C. at 65% relative humidity, or 6 months at 40° C. and 75% relative humidity) and observing no substantial change in the concentration of losartan or a pharmaceutically acceptable salt thereof, or other components in the composition. In some aspects, a pharmaceutical composition is considered “stable” if it exhibits substantially the same characteristics before and after storage at a given temperature and relative humidity over a given time, wherein the characteristics are one or more of: the concentration of losartan or a pharmaceutically acceptable salt thereof, the concentration of an impurity, the appearance of the composition, the viscosity of the composition, the uniformity of the dosage form, or the sedimentation rate of the composition (for suspension products). In some aspects, the pharmaceutical composition is substantially free of impurities following storage.

As used herein, the terms “impurity” and “impurities” refer to any chemical entities (e.g., product of losartan oxidation or degradation) or microbial entities (e.g., a living or dead microbial species) present in a pharmaceutical composition described herein, other than the disclosed components of the pharmaceutical composition. In some aspects, an impurity is a change in the physical properties and/or chemical structure of losartan or a pharmaceutically acceptable salt thereof, due to exposure to heat, oxidation, excipients, and/or time under storage. An impurity can alter the pharmacokinetic properties of the pharmaceutical composition (e.g., where the impurity is a chemical derivative of losartan or a pharmaceutically acceptable salt thereof), the stability of the pharmaceutical composition, or the safety of administration of the pharmaceutical composition (e.g., where the impurity is a pathogenic or pyrogenic microbial contaminant). In some aspects, the impurity is a side product or byproduct of a method of losartan synthesis. In some aspects, an impurity is a product of losartan oxidation or degradation, indicating a decrease in losartan dosage, or formation of a chemical entity having a different bioavailability than losartan. In some aspects, the impurity is a pyrogenic microbial entity or product of a microbial entity.

As used herein, the term “wt/wt %” refers to the percentage, by mass, of a component of a pharmaceutical composition described herein, relative to the mass of the entire pharmaceutical composition, unless otherwise specified (as in the case of “wt/wt % relative to the mass of losartan”). For example, if a component is described as 10 wt/wt %, the mass of the component is 10% of the mass of the entire composition including the component.

As used herein, “wt/wt % relative to the mass of losartan” refers to the percentage, by mass, of a component of a pharmaceutical composition described herein, relative to the mass of losartan present in the pharmaceutical composition. For example, if an impurity is described as “less than 2 wt/wt % relative to the mass of losartan”, the mass of the impurity present in the pharmaceutical composition is less than 2% of the mass of losartan present in the pharmaceutical composition. It will be understood by a person of ordinary skill in the art to which this application pertains that “the mass of losartan” in this context refers to the mass of losartan, and does not include the counterions of a salt of losartan (e.g., if losartan in the form of losartan potassium is added to form a composition).

As used herein, the term “wt/vol %” refers to the percentage, by mass, of a component of a liquid pharmaceutical composition described herein, relative to the volume of the entire liquid pharmaceutical composition, where the ratio corresponds to a number of grams of the component in a 100 mL volume. For example, if a component is described as 10 wt/vol %, the ratio of the mass of the component relative to the volume of the liquid pharmaceutical composition is that of 100 mL of a liquid composition containing 10 grams of the component.

The term “losartan” refers to 2-butyl-4-chloro-1-((2′-(1H-tetrazol-5-yl) (1,1′-biphenyl)-4-yl)methyl)-1H-imidazole-5-methanol, having the following chemical structure:

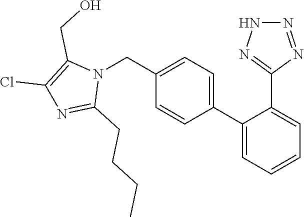 [SMILES: CCCCc1nc(Cl)c(CO)n1Cc1ccc(-c2ccccc2-c2nn[nH]n2)cc1]

The term “losartan impurity D” refers to 2-butyl-4-chloro-5-formylimidazole, also known as 2-butyl-5-chloro-1H-imidazole-4-carboxaldehyde, having the following chemical structure:

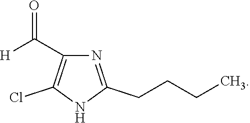 [SMILES: CCCCc1nc(C=O)c(Cl)[nH]1]

The term “losartan impurity E” refers to 5-(4′-methylbiphenyl-2-yl)-1H-tetrazole, having the following chemical structure:

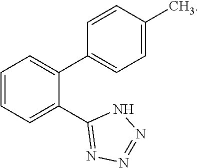 [SMILES: Cc1ccc(-c2ccccc2-c2nnn[nH]2)cc1]

Losartan is marketed as a losartan potassium salt tablet formulation under the trade name COZAAR®.

As used herein, the term “sedimentation rate” refers to the rate at which a suspended phase of a suspension settles to the surface of the container.

As used herein, the term “liquid” when referring to a pharmaceutical composition herein refers to a composition which is substantially a liquid. Liquid compositions of the present disclosure include suspensions, which are substantially liquid, despite comprising solid particles suspended in a liquid phase.

As used herein, the term “multiple emulsion” refers to a liquid composition comprising both oil-in-water and water-in-oil type emulsions.

As used herein, the term “microemulsion” refers to a thermodynamically stable emulsion. In some aspects, a microemulsion is transparent or slightly turbid. In some aspects, the dispersed phase of a microemulsion consists of small droplets with a diameter of about 100 Å to about 1,000 Å.

The terms “co-administration”, “co-administering”, or “co-administered” refer to administering a combination of therapeutic agents, such as, for example, a combination of losartan and an anti-hypertensive drug. The combination can be administered as two separate entities, such as, for example, in separate dosage forms, or as a single combination entity, such as, for example, in the same oral, liquid pharmaceutical composition. One therapeutic agent (e.g., losartan or a pharmaceutically acceptable salt form thereof) can be administered before, concomitantly, or subsequently to the administering of the other therapeutic agent (e.g., an anti-hypertensive drug) to the subject.

### BRIEF DESCRIPTION OF THE DRAWINGS

FIG. 1 shows the plasma concentration of losartan following administration of 5 mg oral dose of losartan potassium in rabbits, as described in Example 15. Corresponding losartan pharmacokinetic parameters are summarized in Table 13.

FIG. 2 shows the plasma concentration of losartan carboxylic acid metabolite following administration of 5 mg oral dose losartan potassium in rabbits, as described in Example 15. Corresponding losartan carboxylic acid pharmacokinetic parameters are summarized in Table 14.

FIG. 3 shows Kaplan-Meier estimates of the primary endpoint of time to cardiovascular death, nonfatal stroke, or nonfatal myocardial infarction in the groups treated with losartan and atenolol according to the study of Example 30. The Risk Reduction is adjusted for baseline Framingham risk score and level of electrocardiogra left ventricular hypertrophy.

FIG. 4 shows Kaplan-Meier estimates of the time to fatal/nonfatal stroke in the groups treated with losartan and atenolol according to the study of Example 30. The Risk Reduction is adjusted for baseline Framingham risk score and level of electrocardiogra left ventricular hypertrophy.

FIG. 5 shows the primary endpoint events within Demographic Subgroups according to the study of Example 30.

FIG. 6 shows a Kaplan-Meier curve for the primary composite endpoint of doubling of serum creatinine, end stage renal disease (need for dialysis or transplantation) or death according to the study of Example 31.

### DETAILED DESCRIPTION OF THE INVENTION

### Pharmaceutical Composition

In some aspects, the present disclosure provides a pharmaceutical composition comprising losartan or a pharmaceutically acceptable salt thereof and one or more pharmaceutically acceptable excipients, wherein the pharmaceutical composition is suitable for oral administration, liquid, substantially free of impurities, and stable for at least 12 months. In some aspects, the pharmaceutical composition is bioequivalent to COZAAR®. In some aspects, the pharmaceutical composition is not bioequivalent to COZAAR®.

In some aspects, the present disclosure provides a pharmaceutical composition comprising losartan or pharmaceutically acceptable salt thereof and one or more pharmaceutically acceptable excipients, wherein the pharmaceutical composition is suitable for oral administration, liquid, bioequivalent to COZAAR®, substantially free of impurities, and stable for at least 12 months.

### Forms of Losartan

In some aspects, the pharmaceutical composition comprises losartan.

In some aspects, the pharmaceutical composition comprises a pharmaceutically acceptable salt of losartan. In some aspects, the pharmaceutical composition comprises losartan potassium. In some aspects, the pharmaceutical composition comprises a sodium salt of losartan. In some aspects, the pharmaceutical composition comprises a lithium salt of losartan.

### Excipients

In some aspects, the pharmaceutical composition comprises one or more pharmaceutically acceptable excipients selected from the group consisting of: a suspending agent, a pH modifying agent, an emulsifying agent, an antioxidant, a chelating agent, a preservative, an antifoaming agent, a solubilizer, a vehicle, a surfactant or wetting agent, a sweetener, a stabilizer, a flavoring agent, and a colorant. In some aspects, the pharmaceutical composition comprises two or more pharmaceutically acceptable excipients selected from the group consisting of: a suspending agent, a pH modifying agent, an emulsifying agent, an antioxidant, a chelating agent, a preservative, an antifoaming agent, a solubilizer, a vehicle, a surfactant or wetting agent, a sweetener, a stabilizer, a flavoring agent, and a colorant.

In some aspects, the pharmaceutical composition comprises a crystallization inhibitor or a crystal growth inhibitor. In some aspects, the crystallization inhibitor or crystal growth inhibitor is a polymer. In some aspects, the polymer is polyvinylpyrrolidone. In some aspects, the polymer is polyvinylpyrrolidone K90. In some aspects, the polymer is polyvinylpyrrolidone K30. In some aspects, the polymer is hydroxypropyl methylcellulose. In some aspects, the polymer is polyvinyl acetate. In some aspects, the polymer is a cyclodextrin. In some aspects, the cyclodextrin is hydroxypropyl β-cyclodextrin.

In some aspects, the one or more excipients comprise a suspending agent. In some aspects, the suspending agent is selected from the group consisting of: hydroxyethylcellulose, methylcellulose, hydroxymethylcellulose, hydroxypropylmethylcellulose, microcrystalline cellulose, sodium carboxymethylcellulose, xanthan gum, acacia, an alginate, and guar gum. In some aspects, the suspending agent is hydroxyethylcellulose.

In some aspects, the suspending agent is methylcellulose. In some aspects, the suspending agent is hydroxymethylcellulose. In some aspects, the suspending agent is hydroxypropylmethylcellulose. In some aspects, the suspending agent is microcrystalline cellulose. In some aspects, the suspending agent is sodium carboxymethylcellulose. In some aspects, the suspending agent is xanthan gum. In some aspects, the suspending agent is acacia. In some aspects, the suspending agent is an alginate. In some aspects, the suspending agent is guar gum. In some aspects, the suspending agent comprises a thickening agent.

In some aspects, the suspending agent is present in an amount of 0.1 to 15 wt/wt %. In some aspects, the suspending agent is present in an amount of 0.5 to 10 wt/wt %. In some aspects, the suspending agent is present in an amount of 1 to 8 wt/wt %. In some aspects, the suspending agent is present in an amount of 2 to 7 wt/wt %. In some aspects, the suspending agent is present in an amount of 3 to 6 wt/wt %. In some aspects, the suspending agent is present in an amount of about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, or about 15 wt/wt %.

In some aspects, the suspending agent is present in an amount of 0.1 to 15 wt/vol %. In some aspects, the suspending agent is present in an amount of 0.5 to 10 wt/vol %. In some aspects, the suspending agent is present in an amount of 1 to 8 wt/vol %. In some aspects, the suspending agent is present in an amount of 2 to 7 wt/vol %. In some aspects, the suspending agent is present in an amount of 3 to 6 wt/vol %. In some aspects, the suspending agent is present in an amount of about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, about 10, about 11, about 12, about 13, about 14, or about 15 wt/vol %.

In some aspects, the one or more excipients comprise a pH modifying agent. In some aspects, the pH modifying agent is selected from the group consisting of: citric acid, sodium citrate, sodium hydroxide, sodium dihydrogen phosphate, and disodium hydrogen phosphate. In some aspects, the pH modifying agent is citric acid. In some aspects, the pH modifying agent is sodium citrate. In some aspects, the pH modifying agent is sodium hydroxide. In some aspects, the pH modifying agent is sodium dihydrogen phosphate. In some aspects, the pH modifying agent is disodium hydrogen phosphate.

In some aspects, the one or more excipients comprise an emulsifying agent selected from the group consisting of: a polysorbate, cetostearyl alcohol, and cetyl alcohol. In some aspects, the emulsifying agent is a polysorbate. In some aspects, the emulsifying agent is cetostearyl alcohol. In some aspects, the emulsifying agent is cetyl alcohol.

In some aspects, the one or more excipients comprise an antioxidant. In some aspects, the antioxidant is selected from the group consisting of: butylated hydroxyanisole, butylated hydroxytoluene, ascorbic acid, tocopherol, and vitamin E. In some aspects, the antioxidant is butylated hydroxyanisole. In some aspects, the antioxidant is butylated hydroxytoluene. In some aspects, the antioxidant is ascorbic acid. In some aspects, the antioxidant is tocopherol. In some aspects, the antioxidant is vitamin E.

In some aspects, the pharmaceutical composition comprises an antioxidant in an amount of less than 10 wt/wt %. In some aspects, the pharmaceutical composition comprises an antioxidant in an amount of less than 9 wt/wt %. In some aspects, the pharmaceutical composition comprises an antioxidant in an amount of less than 8 wt/wt %. In some aspects, the pharmaceutical composition comprises an antioxidant in an amount of less than 7 wt/wt %. In some aspects, the pharmaceutical composition comprises an antioxidant in an amount of less than 6 wt/wt %. In some aspects, the pharmaceutical composition comprises an antioxidant in an amount of less than 5 wt/wt %. In some aspects, the pharmaceutical composition comprises an antioxidant in an amount of less than 4 wt/wt %. In some aspects, the pharmaceutical composition comprises an antioxidant in an amount of less than 3 wt/wt %. In some aspects, the pharmaceutical composition comprises an antioxidant in an amount of less than 2 wt/wt %. In some aspects, the pharmaceutical composition comprises an antioxidant in an amount of less than 1 wt/wt %.

In some aspects, the pharmaceutical composition comprises an antioxidant in an amount of less than 10 wt/vol %. In some aspects, the pharmaceutical composition comprises an antioxidant in an amount of less than 9 wt/vol %. In some aspects, the pharmaceutical composition comprises an antioxidant in an amount of less than 8 wt/vol %. In some aspects, the pharmaceutical composition comprises an antioxidant in an amount of less than 7 wt/vol %. In some aspects, the pharmaceutical composition comprises an antioxidant in an amount of less than 6 wt/vol %. In some aspects, the pharmaceutical composition comprises an antioxidant in an amount of less than 5 wt/vol %. In some aspects, the pharmaceutical composition comprises an antioxidant in an amount of less than 4 wt/vol %. In some aspects, the pharmaceutical composition comprises an antioxidant in an amount of less than 3 wt/vol %. In some aspects, the pharmaceutical composition comprises an antioxidant in an amount of less than 2 wt/vol %. In some aspects, the pharmaceutical composition comprises an antioxidant in an amount of less than 1 wt/vol %.

In some aspects, the one or more excipients comprise a chelating agent. In some aspects, the chelating agent is ethylenediaminetetraacetic acid. In some aspects, the chelating agent is a disodium salt of ethylenediaminetetraacetic acid.

In some aspects, the one or more excipients comprise a preservative. In some aspects, the preservative is selected from the group consisting of: methyl paraben, ethyl paraben, propyl paraben, butyl paraben, benzoic acid, sodium benzoate, and benzalkonium chloride. In some aspects, the preservative is methyl paraben. In some aspects, the preservative is ethyl paraben. In some aspects, the preservative is propyl paraben. In some aspects, the preservative is butyl paraben. In some aspects, the preservative is benzoic acid. In some aspects, the preservative is sodium benzoate. In some aspects, the preservative is benzalkonium chloride.

In some aspects, the pharmaceutical composition comprises a preservative in an amount of less than 10 wt/wt %. In some aspects, the pharmaceutical composition comprises a preservative in an amount of less than 9 wt/wt %. In some aspects, the pharmaceutical composition comprises a preservative in an amount of less than 8 wt/wt %. In some aspects, the pharmaceutical composition comprises a preservative in an amount of less than 7 wt/wt %. In some aspects, the pharmaceutical composition comprises a preservative in an amount of less than 6 wt/wt %. In some aspects, the pharmaceutical composition comprises a preservative in an amount of less than 5 wt/wt %. In some aspects, the pharmaceutical composition comprises a preservative in an amount of less than 4 wt/wt %. In some aspects, the pharmaceutical composition comprises a preservative in an amount of less than 3 wt/wt %. In some aspects, the pharmaceutical composition comprises a preservative in an amount of less than 2 wt/wt %. In some aspects, the pharmaceutical composition comprises a preservative in an amount of less than 1 wt/wt %.

In some aspects, the pharmaceutical composition comprises a preservative in an amount of less than 10 wt/vol %. In some aspects, the pharmaceutical composition comprises a preservative in an amount of less than 9 wt/vol %. In some aspects, the pharmaceutical composition comprises a preservative in an amount of less than 8 wt/vol %. In some aspects, the pharmaceutical composition comprises a preservative in an amount of less than 7 wt/vol %. In some aspects, the pharmaceutical composition comprises a preservative in an amount of less than 6 wt/vol %. In some aspects, the pharmaceutical composition comprises a preservative in an amount of less than 5 wt/vol %. In some aspects, the pharmaceutical composition comprises a preservative in an amount of less than 4 wt/vol %. In some aspects, the pharmaceutical composition comprises a preservative in an amount of less than 3 wt/vol %. In some aspects, the pharmaceutical composition comprises a preservative in an amount of less than 2 wt/vol %. In some aspects, the pharmaceutical composition comprises a preservative in an amount of less than 1 wt/vol %.

In some aspects, the one or more excipients comprise an antifoaming agent. In some aspects, the pharmaceutical composition comprises an antifoaming agent in an amount of less than 10 wt/wt %. In some aspects, the pharmaceutical composition comprises an antifoaming agent in an amount of less than 9 wt/wt %. In some aspects, the pharmaceutical composition comprises an antifoaming agent in an amount of less than 8 wt/wt %. In some aspects, the pharmaceutical composition comprises an antifoaming agent in an amount of less than 7 wt/wt %. In some aspects, the pharmaceutical composition comprises an antifoaming agent in an amount of less than 6 wt/wt %. In some aspects, the pharmaceutical composition comprises an antifoaming agent in an amount of less than 5 wt/wt %. In some aspects, the pharmaceutical composition comprises an antifoaming agent in an amount of less than 4 wt/wt %. In some aspects, the pharmaceutical composition comprises an antifoaming agent in an amount of less than 3 wt/wt %. In some aspects, the pharmaceutical composition comprises an antifoaming agent in an amount of less than 2 wt/wt %. In some aspects, the pharmaceutical composition comprises an antifoaming agent in an amount of less than 1 wt/wt %.

In some aspects, the one or more excipients comprise an antifoaming agent. In some aspects, the pharmaceutical composition comprises an antifoaming agent in an amount of less than 10 wt/vol %. In some aspects, the pharmaceutical composition comprises an antifoaming agent in an amount of less than 9 wt/vol %. In some aspects, the pharmaceutical composition comprises an antifoaming agent in an amount of less than 8 wt/vol %. In some aspects, the pharmaceutical composition comprises an antifoaming agent in an amount of less than 7 wt/vol %. In some aspects, the pharmaceutical composition comprises an antifoaming agent in an amount of less than 6 wt/vol %. In some aspects, the pharmaceutical composition comprises an antifoaming agent in an amount of less than 5 wt/vol %. In some aspects, the pharmaceutical composition comprises an antifoaming agent in an amount of less than 4 wt/vol %. In some aspects, the pharmaceutical composition comprises an antifoaming agent in an amount of less than 3 wt/vol %. In some aspects, the pharmaceutical composition comprises an antifoaming agent in an amount of less than 2 wt/vol %. In some aspects, the pharmaceutical composition comprises an antifoaming agent in an amount of less than 1 wt/vol %.

In some aspects, the one or more excipients comprise a solubilizer. In some aspects, the solubilizer is cremophor. In some aspects, the solubilizer is vitamin E. In some aspects, the solubilizer is polyethylene glycol. In some aspects, the solubilizer is propylene glycol. In some aspects, the solubilizer comprises a co-solvent.

In some aspects, the one or more excipients comprise a vehicle. In some aspects, the vehicle is selected from the group consisting of: water, ethanol, glycerol, propylene glycol, polyethylene glycol, and an oil. In some aspects, the vehicle is water. In some aspects, the vehicle is ethanol. In some aspects, the vehicle is glycerol. In some aspects, the vehicle is propylene glycol. In some aspects, the vehicle is an oil.

In some aspects, the one or more excipients comprise a surfactant or a wetting agent. In some aspects, the surfactant or wetting agent is selected from the group consisting of: a polysorbate, a polyoxamer, and sodium lauryl sulfate. In some aspects, the surfactant or wetting agent is polysorbate. In some aspects, the surfactant or wetting agent is a polyoxamer. In some aspects, the surfactant or wetting agent is sodium lauryl sulfate.

In some aspects, the one or more excipients comprise a sweetener. In some aspects, the sweetener is selected from the list consisting of: sorbitol, mannitol, xylitol, aspartame, sucralose, saccharine, and acesulfame K. In some aspects, the sweetener is sorbitol. In some aspects, the sweetener is mannitol. In some aspects, the sweetener is xylitol. In some aspects, the sweetener is aspartame. In some aspects, the sweetener is sucralose. In some aspects, the sweetener is saccharine. In some aspects, the sweetener is acesulfame K.

In some aspects, the pharmaceutical composition comprises a sweetener in an amount of less than 10 wt/wt %. In some aspects, the pharmaceutical composition comprises a sweetener in an amount of less than 9 wt/wt %. In some aspects, the pharmaceutical composition comprises a sweetener in an amount of less than 8 wt/wt %. In some aspects, the pharmaceutical composition comprises a sweetener in an amount of less than 7 wt/wt %. In some aspects, the pharmaceutical composition comprises a sweetener in an amount of less than 6 wt/wt %. In some aspects, the pharmaceutical composition comprises a sweetener in an amount of less than 5 wt/wt %. In some aspects, the pharmaceutical composition comprises a sweetener in an amount of less than 4 wt/wt %. In some aspects, the pharmaceutical composition comprises a sweetener in an amount of less than 3 wt/wt %. In some aspects, the pharmaceutical composition comprises a sweetener in an amount of less than 2 wt/wt %. In some aspects, the pharmaceutical composition comprises a sweetener in an amount of less than 1 wt/wt %.

In some aspects, the pharmaceutical composition comprises a sweetener in an amount of less than 10 wt/vol %. In some aspects, the pharmaceutical composition comprises a sweetener in an amount of less than 9 wt/vol %. In some aspects, the pharmaceutical composition comprises a sweetener in an amount of less than 8 wt/vol %. In some aspects, the pharmaceutical composition comprises a sweetener in an amount of less than 7 wt/vol %. In some aspects, the pharmaceutical composition comprises a sweetener in an amount of less than 6 wt/vol %. In some aspects, the pharmaceutical composition comprises a sweetener in an amount of less than 5 wt/vol %. In some aspects, the pharmaceutical composition comprises a sweetener in an amount of less than 4 wt/vol %. In some aspects, the pharmaceutical composition comprises a sweetener in an amount of less than 3 wt/vol %. In some aspects, the pharmaceutical composition comprises a sweetener in an amount of less than 2 wt/vol %. In some aspects, the pharmaceutical composition comprises a sweetener in an amount of less than 1 wt/vol %.

In some aspects, the one or more excipients comprise a stabilizer.

In some aspects, the one or more excipients comprise a flavoring agent. In some aspects, the flavoring agent is selected from the list consisting of: an apple flavoring agent, an orange flavoring agent, a mint flavoring agent, a cherry flavoring agent, and a strawberry flavoring agent. In some aspects, the flavoring agent is an apple flavoring agent. In some aspects, the flavoring agent is an orange flavoring agent. In some aspects, the flavoring agent is a strawberry flavoring agent.

In some aspects, the pharmaceutical composition comprises a flavoring agent in an amount of less than 10 wt/wt %. In some aspects, the pharmaceutical composition comprises a flavoring agent in an amount of less than 9 wt/wt %. In some aspects, the pharmaceutical composition comprises a flavoring agent in an amount of less than 8 wt/wt %. In some aspects, the pharmaceutical composition comprises a flavoring agent in an amount of less than 7 wt/wt %. In some aspects, the pharmaceutical composition comprises a flavoring agent in an amount of less than 6 wt/wt %. In some aspects, the pharmaceutical composition comprises a flavoring agent in an amount of less than 5 wt/wt %. In some aspects, the pharmaceutical composition comprises a flavoring agent in an amount of less than 4 wt/wt %. In some aspects, the pharmaceutical composition comprises a flavoring agent in an amount of less than 3 wt/wt %. In some aspects, the pharmaceutical composition comprises a flavoring agent in an amount of less than 2 wt/wt %. In some aspects, the pharmaceutical composition comprises a flavoring agent in an amount of less than 1 wt/wt %.

In some aspects, the pharmaceutical composition comprises a flavoring agent in an amount of less than 10 wt/vol %. In some aspects, the pharmaceutical composition comprises a flavoring agent in an amount of less than 9 wt/vol %. In some aspects, the pharmaceutical composition comprises a flavoring agent in an amount of less than 8 wt/vol %. In some aspects, the pharmaceutical composition comprises a flavoring agent in an amount of less than 7 wt/vol %. In some aspects, the pharmaceutical composition comprises a flavoring agent in an amount of less than 6 wt/vol %. In some aspects, the pharmaceutical composition comprises a flavoring agent in an amount of less than 5 wt/vol %. In some aspects, the pharmaceutical composition comprises a flavoring agent in an amount of less than 4 wt/vol %. In some aspects, the pharmaceutical composition comprises a flavoring agent in an amount of less than 3 wt/vol %. In some aspects, the pharmaceutical composition comprises a flavoring agent in an amount of less than 2 wt/vol %. In some aspects, the pharmaceutical composition comprises a flavoring agent in an amount of less than 1 wt/vol %.

In some aspects, the one or more excipients comprise a colorant.

In some aspects, the pharmaceutical composition comprises 10 mg/mL of losartan potassium, dibasic sodium phosphate, hypromellose, methyl paraben, monobasic sodium phosphate, natural peppermint flavor, polyethylene glycol, povidone, propyl paraben, propylene glycol, purified water, simethicone, sucralose, and xanthan gum. In some aspects, the pharmaceutical composition is a white, translucent oral suspension with a peppermint odor.

Free from Impurities

In some aspects, the pharmaceutical composition is substantially free of impurities. In some aspects, the pharmaceutical composition comprises less than 5 wt/wt % of

all impurities relative to the mass of losartan. In some aspects, the pharmaceutical composition comprises less than 4.5 wt/wt % of all impurities relative to the mass of losartan. In some aspects, the pharmaceutical composition comprises less than 4 wt/wt % of all impurities relative to the mass of losartan. In some aspects, the pharmaceutical composition comprises less than 3.5 wt/wt % of all impurities relative to the mass of losartan. In some aspects, the pharmaceutical composition comprises less than 3 wt/wt % of all impurities relative to the mass of losartan. In some aspects, the pharmaceutical composition comprises less than 2.5 wt/wt % of all impurities relative to the mass of losartan. In some aspects, the pharmaceutical composition comprises less than 2 wt/wt % of all impurities relative to the mass of losartan. In some aspects, the pharmaceutical composition comprises less than 1.5 wt/wt % of all impurities relative to the mass of losartan. In some aspects, the pharmaceutical composition comprises less than 1 wt/wt % of all impurities relative to the mass of losartan. In some aspects, the pharmaceutical composition comprises less than 0.9 wt/wt % of all impurities relative to the mass of losartan. In some aspects, the pharmaceutical composition comprises less than 0.8 wt/wt % of all impurities relative to the mass of losartan. In some aspects, the pharmaceutical composition comprises less than 0.7 wt/wt % of all impurities relative to the mass of losartan. In some aspects, the pharmaceutical composition comprises less than 0.6 wt/wt % of all impurities relative to the mass of losartan. In some aspects, the pharmaceutical composition comprises less than 0.5 wt/wt % of all impurities relative to the mass of losartan. In some aspects, the pharmaceutical composition comprises less than 0.4 wt/wt % of all impurities relative to the mass of losartan. In some aspects, the pharmaceutical composition comprises less than 0.3 wt/wt % of all impurities relative to the mass of losartan. In some aspects, the pharmaceutical composition comprises less than 0.2 wt/wt % of all impurities relative to the mass of losartan. In some aspects, the pharmaceutical composition comprises less than 0.1 wt/wt % of all impurities relative to the mass of losartan. In some aspects, the pharmaceutical composition comprises less than 0.08 wt/wt % of all impurities relative to the mass of losartan. In some aspects, the pharmaceutical composition comprises less than 0.05 wt/wt % of all impurities relative to the mass of losartan. In some aspects, the pharmaceutical composition comprises less than 0.03 wt/wt % of all impurities relative to the mass of losartan. In some aspects, the pharmaceutical composition comprises less than 0.02 wt/wt % of all impurities relative to the mass of losartan. In some aspects, the pharmaceutical composition comprises less than 0.01 wt/wt % of all impurities relative to the mass of losartan.

In some aspects, the pharmaceutical composition comprises less than 5 wt/wt % of an impurity relative to the mass of losartan. In some aspects, the pharmaceutical composition comprises less than 4.5 wt/wt % of an impurity relative to the mass of losartan. In some aspects, the pharmaceutical composition comprises less than 4 wt/wt % of an impurity relative to the mass of losartan. In some aspects, the pharmaceutical composition comprises less than 3.5 wt/wt % of an impurity relative to the mass of losartan. In some aspects, the pharmaceutical composition comprises less than 3 wt/wt % of an impurity relative to the mass of losartan. In some aspects, the pharmaceutical composition comprises less than 2.5 wt/wt % of an impurity relative to the mass of losartan. In some aspects, the pharmaceutical composition comprises less than 2 wt/wt % of an impurity relative to the mass of losartan. In some aspects, the pharmaceutical composition comprises less than 1.5 wt/wt % of an impurity relative to the mass of losartan. In some aspects, the pharmaceutical composition comprises less than 1 wt/wt % of an impurity relative to the mass of losartan. In some aspects, the pharmaceutical composition comprises less than 0.9 wt/wt % of an impurity relative to the mass of losartan. In some aspects, the pharmaceutical composition comprises less than 0.8 wt/wt % of an impurity relative to the mass of losartan. In some aspects, the pharmaceutical composition comprises less than 0.7 wt/wt % of an impurity relative to the mass of losartan. In some aspects, the pharmaceutical composition comprises less than 0.6 wt/wt % of an impurity relative to the mass of losartan. In some aspects, the pharmaceutical composition comprises less than 0.5 wt/wt % of an impurity relative to the mass of losartan. In some aspects, the pharmaceutical composition comprises less than 0.4 wt/wt % of an impurity relative to the mass of losartan. In some aspects, the pharmaceutical composition comprises less than 0.3 wt/wt % of an impurity relative to the mass of losartan. In some aspects, the pharmaceutical composition comprises less than 0.2 wt/wt % of an impurity relative to the mass of losartan. In some aspects, the pharmaceutical composition comprises less than 0.1 wt/wt % of an impurity relative to the mass of losartan. In some aspects, the pharmaceutical composition comprises less than 0.08 wt/wt % of an impurity relative to the mass of losartan. In some aspects, the pharmaceutical composition comprises less than 0.05 wt/wt % of an impurity relative to the mass of losartan. In some aspects, the pharmaceutical composition comprises less than 0.03 wt/wt % of an impurity relative to the mass of losartan. In some aspects, the pharmaceutical composition comprises less than 0.02 wt/wt % of an impurity relative to the mass of losartan. In some aspects, the pharmaceutical composition comprises less than 0.01 wt/wt % of an impurity relative to the mass of losartan.

In some aspect the impurity is a product of losartan oxidation.

In some aspects, the impurity is a product of losartan degradation.

In some aspects, the impurity is a side product of a method of losartan synthesis.

In some aspects, the impurity is a byproduct of a method of losartan synthesis.

In some aspects, the impurity is losartan carboxylic acid.

In some aspects, the impurity is losartan impurity D. In some aspects, the pharmaceutical composition comprises less than 5 wt/wt % of losartan impurity D relative to the mass of losartan. In some aspects, the pharmaceutical composition comprises less than 4.5 wt/wt % of losartan impurity D relative to the mass of losartan. In some aspects, the pharmaceutical composition comprises less than 4 wt/wt % of losartan impurity D relative to the mass of losartan. In some aspects, the pharmaceutical composition comprises less than 3.5 wt/wt % of losartan impurity D relative to the mass of losartan. In some aspects, the pharmaceutical composition comprises less than 3 wt/wt % of losartan impurity D relative to the mass of losartan. In some aspects, the pharmaceutical composition comprises less than 2.5 wt/wt % of losartan impurity D relative to the mass of losartan. In some aspects, the pharmaceutical composition comprises less than 2 wt/wt % of losartan impurity D relative to the mass of losartan. In some aspects, the pharmaceutical composition comprises less than 1.9 wt/wt % of losartan impurity D relative to the mass of losartan. In some aspects, the pharmaceutical composition comprises less than 1.8 wt/wt % of losartan impurity D relative to the mass of losartan. In some aspects, the pharmaceutical composition comprises less than 1.7 wt/wt % of losartan impurity D relative to the mass of losartan. In some aspects, the pharmaceutical composition comprises less than 1.6 wt/wt % of losartan impurity D relative to the mass of losartan. In some aspects, the pharmaceutical composition comprises less than 1.5 wt/wt % of losartan impurity D relative to the mass of losartan. In some aspects, the pharmaceutical composition comprises less than 1.4 wt/wt % of losartan impurity D relative to the mass of losartan. In some aspects, the pharmaceutical composition comprises less than 1.3 wt/wt % of losartan impurity D relative to the mass of losartan. In some aspects, the pharmaceutical composition comprises less than 1.2 wt/wt % of losartan impurity D relative to the mass of losartan. In some aspects, the pharmaceutical composition comprises less than 1.1 wt/wt % of losartan impurity D relative to the mass of losartan. In some aspects, the pharmaceutical composition comprises less than 1 wt/wt % of losartan impurity D relative to the mass of losartan. In some aspects, the pharmaceutical composition comprises less than 0.9 wt/wt % of losartan impurity D relative to the mass of losartan. In some aspects, the pharmaceutical composition comprises less than 0.8 wt/wt % of losartan impurity D relative to the mass of losartan. In some aspects, the pharmaceutical composition comprises less than 0.7 wt/wt % of losartan impurity D relative to the mass of losartan. In some aspects, the pharmaceutical composition comprises less than 0.6 wt/wt % of losartan impurity D relative to the mass of losartan. In some aspects, the pharmaceutical composition comprises less than 0.5 wt/wt % of losartan impurity D relative to the mass of losartan. In some aspects, the pharmaceutical composition comprises less than 0.4 wt/wt % of losartan impurity D relative to the mass of losartan. In some aspects, the pharmaceutical composition comprises less than 0.3 wt/wt % of losartan impurity D relative to the mass of losartan. In some aspects, the pharmaceutical composition comprises less than 0.2 wt/wt % of losartan impurity D relative to the mass of losartan. In some aspects, the pharmaceutical composition comprises less than 0.1 wt/wt % of losartan impurity D relative to the mass of losartan. In some aspects, the pharmaceutical composition comprises less than 0.05 wt/wt % of losartan impurity D relative to the mass of losartan. In some aspects, the pharmaceutical composition comprises less than 0.01 wt/wt % of losartan impurity D relative to the mass of losartan.

In some aspects, the impurity is losartan impurity E. In some aspects, the pharmaceutical composition comprises less than 5 wt/wt % of losartan impurity E relative to the mass of losartan. In some aspects, the pharmaceutical composition comprises less than 4.5 wt/wt % of losartan impurity E relative to the mass of losartan. In some aspects, the pharmaceutical composition comprises less than 4 wt/wt % of losartan impurity E relative to the mass of losartan. In some aspects, the pharmaceutical composition comprises less than 3.5 wt/wt % of losartan impurity E relative to the mass of losartan. In some aspects, the pharmaceutical composition comprises less than 3 wt/wt % of losartan impurity E relative to the mass of losartan. In some aspects, the pharmaceutical composition comprises less than 2.5 wt/wt % of losartan impurity E relative to the mass of losartan. In some aspects, the pharmaceutical composition comprises less than 2 wt/wt % of losartan impurity E relative to the mass of losartan. In some aspects, the pharmaceutical composition comprises less than 1.9 wt/wt % of losartan impurity E relative to the mass of losartan. In some aspects, the pharmaceutical composition comprises less than 1.8 wt/wt % of losartan impurity E relative to the mass of losartan. In some aspects, the pharmaceutical composition comprises less than 1.7 wt/wt % of losartan impurity E relative to the mass of losartan. In some aspects, the pharmaceutical composition comprises less than 1.6 wt/wt % of losartan impurity E relative to the mass of losartan. In some aspects, the pharmaceutical composition comprises less than 1.5 wt/wt % of losartan impurity E relative to the mass of losartan. In some aspects, the pharmaceutical composition comprises less than 1.4 wt/wt % of losartan impurity E relative to the mass of losartan. In some aspects, the pharmaceutical composition comprises less than 1.3 wt/wt % of losartan impurity E relative to the mass of losartan. In some aspects, the pharmaceutical composition comprises less than 1.2 wt/wt % of losartan impurity E relative to the mass of losartan. In some aspects, the pharmaceutical composition comprises less than 1.1 wt/wt % of losartan impurity E relative to the mass of losartan. In some aspects, the pharmaceutical composition comprises less than 1 wt/wt % of losartan impurity E relative to the mass of losartan. In some aspects, the pharmaceutical composition comprises less than 0.9 wt/wt % of losartan impurity E relative to the mass of losartan. In some aspects, the pharmaceutical composition comprises less than 0.8 wt/wt % of losartan impurity E relative to the mass of losartan. In some aspects, the pharmaceutical composition comprises less than 0.7 wt/wt % of losartan impurity E relative to the mass of losartan. In some aspects, the pharmaceutical composition comprises less than 0.6 wt/wt % of losartan impurity E relative to the mass of losartan. In some aspects, the pharmaceutical composition comprises less than 0.5 wt/wt % of losartan impurity E relative to the mass of losartan. In some aspects, the pharmaceutical composition comprises less than 0.4 wt/wt % of losartan impurity E relative to the mass of losartan. In some aspects, the pharmaceutical composition comprises less than 0.3 wt/wt % of losartan impurity E relative to the mass of losartan. In some aspects, the pharmaceutical composition comprises less than 0.2 wt/wt % of losartan impurity E relative to the mass of losartan. In some aspects, the pharmaceutical composition comprises less than 0.1 wt/wt % of losartan impurity E relative to the mass of losartan. In some aspects, the pharmaceutical composition comprises less than 0.05 wt/wt % of losartan impurity E relative to the mass of losartan. In some aspects, the pharmaceutical composition comprises less than 0.01 wt/wt % of losartan impurity E relative to the mass of losartan.

In some aspects, the impurity is a biological contaminant. In some aspects, the impurity is a living biological contaminant. In some aspects, the impurity is a dead biological contaminant. In some aspects, the impurity is a viral contaminant. In some aspects, the impurity is a fungal contaminant. In some aspects, the impurity is a bacterial contaminant. In some aspects, the impurity is a bacterial endotoxin.

In some aspects, the biological contaminant is a replication-competent, metabolically inactive or minimally active biological product. In some aspects, the replication-competent, metabolically inactive or minimally active biological product is a spore.

In some aspects, the impurity is pyrogenic.

In some aspects, the impurity is detectable as visible particulate matter.

### Sterility

In some aspects, the pharmaceutical composition has been subjected to a sterilizing treatment. In some aspects, one or more components of the pharmaceutical composition have been subjected to a sterilizing treatment.

In some aspects, the sterilizing treatment comprises filtration. In some aspects, the sterilizing treatment comprises exposure to a high temperature. In some aspects, the sterilizing treatment comprises exposure to a low temperature. In some aspects, the sterilizing treatment comprises application of ultraviolet radiation. In some aspects, the sterilizing treatment is sufficient to kill a living biological contaminant.

### Stability

In some aspects, the pharmaceutically acceptable composition is stable as measured by one or more substantially unchanged characteristics after storage, the one or more substantially unchanged characteristics selected from the group consisting of: the concentration of losartan in the pharmaceutical composition; the concentration of an impurity in the pharmaceutical composition; the visual appearance of the pharmaceutical composition; the viscosity of the pharmaceutical composition; the uniformity of the pharmaceutical composition; and the sedimentation rate of the pharmaceutical composition. In some aspects, the one or more substantially unchanged characteristics comprise the concentration of losartan in the pharmaceutical composition. In some aspects, the one or more substantially unchanged characteristics comprise the concentration of an impurity in the pharmaceutical composition. In some aspects, the one or more substantially unchanged characteristics comprise the visual appearance of the pharmaceutical composition. In some aspects, the one or more substantially unchanged characteristics comprise the viscosity of the pharmaceutical composition. In some aspects, the one or more substantially unchanged characteristics comprise the uniformity of the pharmaceutical composition. In some aspects, the one or more substantially unchanged characteristics comprise the sedimentation rate of the pharmaceutical composition.

In some aspects, the pharmaceutical composition is stable at 2° C.-8° C. and ≤65% relative humidity.

In some aspects, the pharmaceutical composition is stable for 1-24 months at 2° C.-8° C. and ≤65% relative humidity. In some aspects, the pharmaceutical composition is stable for 1-18 months at 2° C.-8° C. and ≤65% relative humidity. In some aspects, the pharmaceutical composition is stable for 1-12 months at 2° C.-8° C. and ≤65% relative humidity. In some aspects, the pharmaceutical composition is stable for 1-6 months at 2° C.-8° C. and ≤65% relative humidity. In some aspects, the pharmaceutical composition is stable for 2-6 months at 2° C.-8° C. and ≤65% relative humidity. In some aspects, the pharmaceutical composition is stable for 3-6 months at 2° C.-8° C. and ≤65% relative humidity. In some aspects, the pharmaceutical composition is stable for 4-6 months at 2° C.-8° C. and ≤65% relative humidity. In some aspects, the pharmaceutical composition is stable for 4-7 months at 2° C.-8° C. and ≤65% relative humidity. In some aspects, the pharmaceutical composition is stable for 4-8 months at 2° C.-8° C. and ≤65% relative humidity. In some aspects, the pharmaceutical composition is stable for 4-10 months at 2° C.-8° C. and ≤65% relative humidity. In some aspects, the pharmaceutical composition is stable for 4-12 months at 2° C.-8° C. and ≤65% relative humidity. In some aspects, the pharmaceutical composition is stable for 4-18 months at 2° C.-8° C. and ≤65% relative humidity. In some aspects, the pharmaceutical composition is stable for 4-24 months at 2° C.-8° C. and ≤65% relative humidity. In some aspects, the pharmaceutical composition is stable for 6-8 months at 2° C.-8° C. and ≤65% relative humidity. In some aspects, the pharmaceutical composition is stable for 6-10 months at 2° C.-8° C. and ≤65% relative humidity. In some aspects, the pharmaceutical composition is stable for 6-12 months at 2° C.-8° C. and ≤65% relative humidity. In some aspects, the pharmaceutical composition is stable for 6-18 months at 2° C.-8° C. and ≤65% relative humidity. In some aspects, the pharmaceutical composition is stable for 6-24 months at 2° C.-8° C. and ≤65% relative humidity. In some aspects, the pharmaceutical composition is stable for 18-24 months at 2° C.-8° C. and ≤65% relative humidity. In some aspects, the pharmaceutical composition is stable for 16-26 months at 2° C.-8° C. and ≤65% relative humidity. In some aspects, the pharmaceutical composition is stable for 18-36 months at 2° C.-8° C. and ≤65% relative humidity. In some aspects, the pharmaceutical composition is stable for 18-30 months at 2° C.-8° C. and ≤65% relative humidity.

In some aspects, the pharmaceutical composition is stable for 2 months at 2° C.-8° C. and ≤65% relative humidity. In some aspects, the pharmaceutical composition is stable for 3 months at 2° C.-8° C. and ≤65% relative humidity. In some aspects, the pharmaceutical composition is stable for 4 months at 2° C.-8° C. and ≤65% relative humidity. In some aspects, the pharmaceutical composition is stable for 5 months at 2° C.-8° C. and ≤65% relative humidity. In some aspects, the pharmaceutical composition is stable for 6 months at 2° C.-8° C. and ≤65% relative humidity. In some aspects, the pharmaceutical composition is stable for 7 months at 2° C.-8° C. and ≤65% relative humidity. In some aspects, the pharmaceutical composition is stable for 8 months at 2° C.-8° C. and ≤65% relative humidity. In some aspects, the pharmaceutical composition is stable for 9 months at 2° C.-8° C. and ≤65% relative humidity. In some aspects, the pharmaceutical composition is stable for 10 months at 2° C.-8° C. and ≤65% relative humidity. In some aspects, the pharmaceutical composition is stable for 11 months at 2° C.-8° C. and ≤65% relative humidity. In some aspects, the pharmaceutical composition is stable for 12 months at 2° C.-8° C. and ≤65% relative humidity. In some aspects, the pharmaceutical composition is stable for 16 months at 2° C.-8° C. and ≤65% relative humidity. In some aspects, the pharmaceutical composition is stable for 18 months at 2° C.-8° C. and ≤65% relative humidity. In some aspects, the pharmaceutical composition is stable for 19 months at 2° C.-8° C. and ≤65% relative humidity. In some aspects, the pharmaceutical composition is stable for 20 months at 2° C.-8° C. and ≤65% relative humidity. In some aspects, the pharmaceutical composition is stable for 21 months at 2° C.-8° C. and ≤65% relative humidity. In some aspects, the pharmaceutical composition is stable for 22 months at 2° C.-8° C. and ≤65% relative humidity. In some aspects, the pharmaceutical composition is stable for 23 months at 2° C.-8° C. and ≤65% relative humidity. In some aspects, the pharmaceutical composition is stable for 24 months at 2° C.-8° C. and ≤65% relative humidity. In some aspects, the pharmaceutical composition is stable for 25 months at 2° C.-8° C. and ≤65% relative humidity. In some aspects, the pharmaceutical composition is stable for 26 months at 2° C.-8° C. and ≤65% relative humidity. In some aspects, the pharmaceutical composition is stable for 27 months at 2° C.-8° C. and ≤65% relative humidity. In some aspects, the pharmaceutical composition is stable for 28 months at 2° C.-8° C. and ≤65% relative humidity. In some aspects, the pharmaceutical composition is stable for 29 months at 2° C.-8° C. and ≤65% relative humidity. In some aspects, the pharmaceutical composition is stable for 30 months at 2° C.-8° C. and ≤65% relative humidity. In some aspects, the pharmaceutical composition is stable for 36 months at 2° C.-8° C. and ≤65% relative humidity.

In some aspects, the pharmaceutical composition is stable at 15° C.-25° C. and ≤65% relative humidity.

In some aspects, the pharmaceutical composition is stable for 1-24 months at 15° C.-25° C. and ≤65% relative humidity. In some aspects, the pharmaceutical composition is stable for 1-18 months at 15° C.-25° C. and ≤65% relative humidity. In some aspects, the pharmaceutical composition is stable for 1-12 months at 15° C.-25° C. and ≤65% relative humidity. In some aspects, the pharmaceutical composition is stable for 1-6 months at 15° C.-25° C. and ≤65% relative humidity. In some aspects, the pharmaceutical composition is stable for 2-6 months at 15° C.-25° C. and ≤65% relative humidity. In some aspects, the pharmaceutical composition is stable for 3-6 months at 15° C.-25° C. and ≤65% relative humidity. In some aspects, the pharmaceutical composition is stable for 4-6 months at 15° C.-25° C. and ≤65% relative humidity. In some aspects, the pharmaceutical composition is stable for 4-7 months at 15° C.-25° C. and ≤65% relative humidity. In some aspects, the pharmaceutical composition is stable for 4-8 months at 15° C.-25° C. and ≤65% relative humidity. In some aspects, the pharmaceutical composition is stable for 4-10 months at 15° C.-25° C. and ≤65% relative humidity. In some aspects, the pharmaceutical composition is stable for 4-12 months at 15° C.-25° C. and ≤65% relative humidity. In some aspects, the pharmaceutical composition is stable for 4-18 months at 15° C.-25° C. and ≤65% relative humidity. In some aspects, the pharmaceutical composition is stable for 4-24 months at 15° C.-25° C. and ≤65% relative humidity. In some aspects, the pharmaceutical composition is stable for 6-8 months at 15° C.-25° C. and ≤65% relative humidity. In some aspects, the pharmaceutical composition is stable for 6-10 months at 15° C.-25° C. and ≤65% relative humidity. In some aspects, the pharmaceutical composition is stable for 6-12 months at 15° C.-25° C. and ≤65% relative humidity. In some aspects, the pharmaceutical composition is stable for 6-18 months at 15° C.-25° C. and ≤65% relative humidity. In some aspects, the pharmaceutical composition is stable for 6-24 months at 15° C.-25° C. and ≤65% relative humidity. In some aspects, the pharmaceutical composition is stable for 18-24 months at 15° C.-25° C. and ≤65% relative humidity. In some aspects, the pharmaceutical composition is stable for 16-26 months at 15° C.-25° C. and ≤65% relative humidity. In some aspects, the pharmaceutical composition is stable for 18-36 months at 15° C.-25° C. and ≤65% relative humidity. In some aspects, the pharmaceutical composition is stable for 18-30 months at 15° C.-25° C. and ≤65% relative humidity.

In some aspects, the pharmaceutical composition is stable for 2 months at 15° C.-25° C. and ≤65% relative humidity. In some aspects, the pharmaceutical composition is stable for 3 months at 15° C.-25° C. and ≤65% relative humidity. In some aspects, the pharmaceutical composition is stable for 4 months at 15° C.-25° C. and ≤65% relative humidity. In some aspects, the pharmaceutical composition is stable for 5 months at 15° C.-25° C. and ≤65% relative humidity. In some aspects, the pharmaceutical composition is stable for 6 months at 15° C.-25° C. and ≤65% relative humidity. In some aspects, the pharmaceutical composition is stable for 7 months at 15° C.-25° C. and ≤65% relative humidity. In some aspects, the pharmaceutical composition is stable for 8 months at 15° C.-25° C. and ≤65% relative humidity. In some aspects, the pharmaceutical composition is stable for 9 months at 15° C.-25° C. and ≤65% relative humidity. In some aspects, the pharmaceutical composition is stable for 10 months at 15° C.-25° C. and ≤65% relative humidity. In some aspects, the pharmaceutical composition is stable for 11 months at 15° C.-25° C. and ≤65% relative humidity. In some aspects, the pharmaceutical composition is stable for 12 months at 15° C.-25° C. and ≤65% relative humidity. In some aspects, the pharmaceutical composition is stable for 16 months at 15° C.-25° C. and ≤65% relative humidity. In some aspects, the pharmaceutical composition is stable for 18 months at 15° C.-25° C. and ≤65% relative humidity. In some aspects, the pharmaceutical composition is stable for 19 months at 15° C.-25° C. and ≤65% relative humidity. In some aspects, the pharmaceutical composition is stable for 20 months at 15° C.-25° C. and ≤65% relative humidity. In some aspects, the pharmaceutical composition is stable for 21 months at 15° C.-25° C. and ≤65% relative humidity. In some aspects, the pharmaceutical composition is stable for 22 months at 15° C.-25° C. and ≤65% relative humidity. In some aspects, the pharmaceutical composition is stable for 23 months at 15° C.-25° C. and ≤65% relative humidity. In some aspects, the pharmaceutical composition is stable for 24 months at 15° C.-25° C. and ≤65% relative humidity. In some aspects, the pharmaceutical composition is stable for 25 months at 15° C.-25° C. and ≤65% relative humidity. In some aspects, the pharmaceutical composition is stable for 26 months at 15° C.-25° C. and ≤65% relative humidity. In some aspects, the pharmaceutical composition is stable for 27 months at 15° C.-25° C. and ≤65% relative humidity. In some aspects, the pharmaceutical composition is stable for 28 months at 15° C.-25° C. and ≤65% relative humidity. In some aspects, the pharmaceutical composition is stable for 29 months at 15° C.-25° C. and ≤65% relative humidity. In some aspects, the pharmaceutical composition is stable for 30 months at 15° C.-25° C. and ≤65% relative humidity. In some aspects, the pharmaceutical composition is stable for 36 months at 15° C.-25° C. and ≤65% relative humidity.

In some aspects, the pharmaceutical composition is stable at 30° C. and ≤65% relative humidity.

In some aspects, the pharmaceutical composition is stable for 1-24 months at 30° C. and ≤65% relative humidity. In some aspects, the pharmaceutical composition is stable for 1-18 months at 30° C. and ≤65% relative humidity. In some aspects, the pharmaceutical composition is stable for 1-12 months at 30° C. and ≤65% relative humidity. In some aspects, the pharmaceutical composition is stable for 1-6 months at 30° C. and ≤65% relative humidity. In some aspects, the pharmaceutical composition is stable for 2-6 months at 30° C. and ≤65% relative humidity. In some aspects, the pharmaceutical composition is stable for 3-6 months at 30° C. and ≤65% relative humidity. In some aspects, the pharmaceutical composition is stable for 4-6 months at 30° C. and ≤65% relative humidity. In some aspects, the pharmaceutical composition is stable for 4-7 months at 30° C. and ≤65% relative humidity. In some aspects, the pharmaceutical composition is stable for 4-8 months at 30° C. and ≤65% relative humidity. In some aspects, the pharmaceutical composition is stable for 4-10 months at 30° C. and ≤65% relative humidity. In some aspects, the pharmaceutical composition is stable for 4-12 months at 30° C. and ≤65% relative humidity. In some aspects, the pharmaceutical composition is stable for 4-18 months at 30° C. and ≤65% relative humidity. In some aspects, the pharmaceutical composition is stable for 4-24 months at 30° C. and ≤65% relative humidity. In some aspects, the pharmaceutical composition is stable for 6-8 months at 30° C. and ≤65% relative humidity. In some aspects, the pharmaceutical composition is stable for 6-10 months at 30° C. and ≤65% relative humidity. In some aspects, the pharmaceutical composition is stable for 6-12 months at 30° C. and ≤65% relative humidity. In some aspects, the pharmaceutical composition is stable for 6-18 months at 30° C. and ≤65% relative humidity. In some aspects, the pharmaceutical composition is stable for 6-24 months at 30° C. and ≤65% relative humidity. In some aspects, the pharmaceutical composition is stable for 18-24 months at 30° C. and ≤65% relative humidity. In some aspects, the pharmaceutical composition is stable for 16-26 months at 30° C. and ≤65% relative humidity. In some aspects, the pharmaceutical composition is stable for 18-36 months at 30° C. and ≤65% relative humidity. In some aspects, the pharmaceutical composition is stable for 18-30 months at 30° C. and ≤65% relative humidity.

In some aspects, the pharmaceutical composition is stable for 2 months at 30° C. and ≤65% relative humidity. In some aspects, the pharmaceutical composition is stable for 3 months at 30° C. and ≤65% relative humidity. In some aspects, the pharmaceutical composition is stable for 4 months at 30° C. and ≤65% relative humidity. In some aspects, the pharmaceutical composition is stable for 5 months at 30° C. and ≤65% relative humidity. In some aspects, the pharmaceutical composition is stable for 6 months at 30° C. and ≤65% relative humidity. In some aspects, the pharmaceutical composition is stable for 7 months at 30° C. and ≤65% relative humidity. In some aspects, the pharmaceutical composition is stable for 8 months at 30° C. and ≤65% relative humidity. In some aspects, the pharmaceutical composition is stable for 9 months at 30° C. and ≤65% relative humidity. In some aspects, the pharmaceutical composition is stable for 10 months at 30° C. and ≤65% relative humidity. In some aspects, the pharmaceutical composition is stable for 11 months at 30° C. and ≤65% relative humidity. In some aspects, the pharmaceutical composition is stable for 12 months at 30° C. and ≤65% relative humidity. In some aspects, the pharmaceutical composition is stable for 16 months at 30° C. and ≤65% relative humidity. In some aspects, the pharmaceutical composition is stable for 18 months at 30° C. and ≤65% relative humidity. In some aspects, the pharmaceutical composition is stable for 19 months at 30° C. and ≤65% relative humidity. In some aspects, the pharmaceutical composition is stable for 20 months at 30° C. and ≤65% relative humidity. In some aspects, the pharmaceutical composition is stable for 21 months at 30° C. and ≤65% relative humidity. In some aspects, the pharmaceutical composition is stable for 22 months at 30° C. and ≤65% relative humidity. In some aspects, the pharmaceutical composition is stable for 23 months at 30° C. and ≤65% relative humidity. In some aspects, the pharmaceutical composition is stable for 24 months at 30° C. and ≤65% relative humidity. In some aspects, the pharmaceutical composition is stable for 25 months at 30° C. and ≤65% relative humidity. In some aspects, the pharmaceutical composition is stable for 26 months at 30° C. and ≤65% relative humidity. In some aspects, the pharmaceutical composition is stable for 27 months at 30° C. and ≤65% relative humidity. In some aspects, the pharmaceutical composition is stable for 28 months at 30° C. and ≤65% relative humidity. In some aspects, the pharmaceutical composition is stable for 29 months at 30° C. and ≤65% relative humidity. In some aspects, the pharmaceutical composition is stable for 30 months at 30° C. and ≤65% relative humidity. In some aspects, the pharmaceutical composition is stable for 36 months at 30° C. and ≤65% relative humidity.

In some aspects, the pharmaceutical composition is stable at 40° C. and 75% relative humidity.

In some aspects, the pharmaceutical composition is stable for 1-24 months at 40° C. and 75% relative humidity. In some aspects, the pharmaceutical composition is stable for 1-18 months at 40° C. and 75% relative humidity. In some aspects, the pharmaceutical composition is stable for 1-12 months at 40° C. and 75% relative humidity. In some aspects, the pharmaceutical composition is stable for 1-6 months at 40° C. and 75% relative humidity. In some aspects, the pharmaceutical composition is stable for 2-6 months at 40° C. and 75% relative humidity. In some aspects, the pharmaceutical composition is stable for 3-6 months at 40° C. and 75% relative humidity. In some aspects, the pharmaceutical composition is stable for 4-6 months at 40° C. and 75% relative humidity. In some aspects, the pharmaceutical composition is stable for 4-7 months at 40° C. and 75% relative humidity. In some aspects, the pharmaceutical composition is stable for 4-8 months at 40° C. and 75% relative humidity. In some aspects, the pharmaceutical composition is stable for 4-10 months at 40° C. and 75% relative humidity. In some aspects, the pharmaceutical composition is stable for 4-12 months at 40° C. and 75% relative humidity. In some aspects, the pharmaceutical composition is stable for 4-18 months at 40° C. and 75% relative humidity. In some aspects, the pharmaceutical composition is stable for 4-24 months at 40° C. and 75% relative humidity. In some aspects, the pharmaceutical composition is stable for 6-8 months at 40° C. and 75% relative humidity. In some aspects, the pharmaceutical composition is stable for 6-10 months at 40° C. and 75% relative humidity. In some aspects, the pharmaceutical composition is stable for 6-12 months at 40° C. and 75% relative humidity. In some aspects, the pharmaceutical composition is stable for 6-18 months at 40° C. and 75% relative humidity. In some aspects, the pharmaceutical composition is stable for 6-24 months at 40° C. and 75% relative humidity. In some aspects, the pharmaceutical composition is stable for 18-24 months at 40° C. and ≤65% relative humidity. In some aspects, the pharmaceutical composition is stable for 16-26 months at 40° C. and ≤65% relative humidity. In some aspects, the pharmaceutical composition is stable for 18-36 months at 40° C. and ≤65% relative humidity. In some aspects, the pharmaceutical composition is stable for 18-30 months at 40° C. and ≤65% relative humidity.

In some aspects, the pharmaceutical composition is stable at 40° C. and 75% relative humidity. In some aspects, the pharmaceutical composition is stable for 2 months at 40° C. and 75% relative humidity. In some aspects, the pharmaceutical composition is stable for 3 months at 40° C. and 75% relative humidity. In some aspects, the pharmaceutical composition is stable for 4 months at 40° C. and 75% relative humidity. In some aspects, the pharmaceutical composition is stable for 5 months at 40° C. and 75% relative humidity. In some aspects, the pharmaceutical composition is stable for 6 months at 40° C. and 75% relative humidity. In some aspects, the pharmaceutical composition is stable for 7 months at 40° C. and 75% relative humidity. In some aspects, the pharmaceutical composition is stable for 8 months at 40° C. and 75% relative humidity. In some aspects, the pharmaceutical composition is stable for 9 months at 40° C. and 75% relative humidity. In some aspects, the pharmaceutical composition is stable for 10 months at 40° C. and 75% relative humidity. In some aspects, the pharmaceutical composition is stable for 11 months at 40° C. and 75% relative humidity. In some aspects, the pharmaceutical composition is stable for 12 months at 40° C. and 75% relative humidity. In some aspects, the pharmaceutical composition is stable for 16 months at 40° C. and 75% relative humidity. In some aspects, the pharmaceutical composition is stable for 18 months at 40° C. and 75% relative humidity. In some aspects, the pharmaceutical composition is stable for 19 months at 40° C. and ≤65% relative humidity. In some aspects, the pharmaceutical composition is stable for 20 months at 40° C. and ≤65% relative humidity. In some aspects, the pharmaceutical composition is stable for 21 months at 40° C. and ≤65% relative humidity. In some aspects, the pharmaceutical composition is stable for 22 months at 40° C. and ≤65% relative humidity.

In some aspects, the pharmaceutical composition is stable for 23 months at 40° C. and ≤65% relative humidity. In some aspects, the pharmaceutical composition is stable for 24 months at 40° C. and ≤65% relative humidity. In some aspects, the pharmaceutical composition is stable for 25 months at 40° C. and ≤65% relative humidity. In some aspects, the pharmaceutical composition is stable for 26 months at 40° C. and ≤65% relative humidity. In some aspects, the pharmaceutical composition is stable for 27 months at 40° C. and ≤65% relative humidity. In some aspects, the pharmaceutical composition is stable for 28 months at 40° C. and ≤65% relative humidity. In some aspects, the pharmaceutical composition is stable for 29 months at 40° C. and ≤65% relative humidity. In some aspects, the pharmaceutical composition is stable for 30 months at 40° C. and ≤65% relative humidity. In some aspects, the pharmaceutical composition is stable for 36 months at 40° C. and 75% relative humidity.

### Liquid and Powder Forms

In some aspects, the pharmaceutical composition of the present disclosure is a solution, an emulsion, or a suspension. In some aspects, the present disclosure provides for a concentrated form of losartan or a pharmaceutically acceptable salt thereof, which can be combined with a suitable diluent to prepare a pharmaceutical composition described herein. In some aspects, the concentrated form of losartan or a pharmaceutically acceptable salt thereof is selected from the group consisting of: a powder, a plurality of granules, modified losartan, and a concentrated liquid form (e.g., a concentrated solution or a low-volume mixed solid and liquid phase). In some aspects, the concentrated form of losartan or a pharmaceutically acceptable salt thereof can be combined with a suitable diluent to prepare one or more of a solution, a suspension, and an emulsion.

In some aspects, the concentrated form of losartan or a pharmaceutically acceptable salt thereof is combined with the suitable diluent and heated to form a pharmaceutically acceptable composition disclosed herein. In some aspects, the concentrated form of losartan or a pharmaceutically acceptable salt thereof is combined with the suitable diluent and an additional component to form a pharmaceutically acceptable composition disclosed herein. In some aspects, the additional component is a pH modifying agent. In some aspects, the pH modifying agent is an acid. In some aspects, the pH modifying agent is a base.

### Solution

In some aspects, the pharmaceutical composition is a solution. In some aspects, the pharmaceutical composition is a solution and the pharmaceutical composition comprises losartan. In some aspects, the pharmaceutical composition is a solution and the pharmaceutical composition comprises a pharmaceutically acceptable salt of losartan. In some aspects, the pharmaceutical composition is a solution and the pharmaceutical composition comprises losartan potassium.

In some aspects, the pharmaceutical composition is a solution comprising losartan substantially dissolved in a pharmaceutically acceptable vehicle. In some aspects, the solution comprises at least 50% of losartan or pharmaceutically acceptable salt thereof in dissolved form. In some aspects, the solution comprises at least 55% of losartan or pharmaceutically acceptable salt thereof in dissolved form. In some aspects, the solution comprises at least 60% of losartan or pharmaceutically acceptable salt thereof in dissolved form. In some aspects, the solution comprises at least 65% of losartan or pharmaceutically acceptable salt thereof in dissolved form. In some aspects, the solution comprises at least 70% of losartan or pharmaceutically acceptable salt thereof in dissolved form. In some aspects, the solution comprises at least 75% of losartan or pharmaceutically acceptable salt thereof in dissolved form. In some aspects, the solution comprises at least 80% of losartan or pharmaceutically acceptable salt thereof in dissolved form. In some aspects, the solution comprises at least 85% of losartan or pharmaceutically acceptable salt thereof in dissolved form. In some aspects, the solution comprises at least 90% of losartan or pharmaceutically acceptable salt thereof in dissolved form. In some aspects, the solution comprises at least 95% of losartan or pharmaceutically acceptable salt thereof in dissolved form. In some aspects, the solution further comprises one or more inactive ingredients selected from the group consisting of: co-solvents, pH modifying agents, surfactants, antioxidants, preservatives, and flavoring agents.

### Emulsion

In some aspects, the pharmaceutical composition is an emulsion. In some aspects, the pharmaceutical composition is an emulsion and the pharmaceutical composition comprises losartan. In some aspects, the pharmaceutical composition is an emulsion and the pharmaceutical composition comprises a pharmaceutically acceptable salt of losartan.

In some aspects, the pharmaceutical composition is an emulsion and the pharmaceutical composition comprises losartan potassium.

In some aspects, the emulsion is a multiple emulsion. In some aspect, the emulsion is a microemulsion.

In some aspects, the emulsion comprises at least two immiscible liquid phases wherein one liquid phase is in the form of globules. In some aspects, the pharmaceutical composition comprises an emulsifying agent. In some aspects, the pharmaceutical composition is an emulsion wherein a hydrophobic phase comprises at least 50% of losartan or a pharmaceutically acceptable salt thereof. In some aspects, the pharmaceutical composition is an emulsion wherein a hydrophobic phase comprises at least 60% of losartan or a pharmaceutically acceptable salt thereof. In some aspects, the pharmaceutical composition is an emulsion wherein a hydrophobic phase comprises at least 70% of losartan or a pharmaceutically acceptable salt thereof. In some aspects, the pharmaceutical composition is an emulsion wherein a hydrophobic phase comprises at least 80% of losartan or a pharmaceutically acceptable salt thereof. In some aspects, the pharmaceutical composition is an emulsion wherein a hydrophobic phase comprises at least 90% of losartan or a pharmaceutically acceptable salt thereof. In some aspects, the pharmaceutical composition is an emulsion wherein a hydrophobic phase comprises at least 95% of losartan or a pharmaceutically acceptable salt thereof.

### Suspension

In some aspects, the pharmaceutical composition is a suspension. In some aspects, the pharmaceutical composition is a suspension and the pharmaceutical composition comprises losartan. In some aspects, the pharmaceutical composition is a suspension and the pharmaceutical composition comprises a pharmaceutically acceptable salt of losartan. In some aspects, the pharmaceutical composition is a suspension and the pharmaceutical composition comprises losartan potassium.

In some aspects, the suspension is a cloudy biphasic liquid comprising a plurality of losartan particles uniformly dispersed in a pharmaceutically acceptable vehicle. In some aspects, at least 50% of losartan or pharmaceutically acceptable salt thereof is in suspended form. In some aspects, at least 60% of losartan or pharmaceutically acceptable salt thereof is in suspended form. In some aspects, at least 70% of losartan or pharmaceutically acceptable salt thereof is in suspended form. In some aspects, at least 80% of losartan or pharmaceutically acceptable salt thereof is in suspended form. In some aspects, at least 90% of losartan or pharmaceutically acceptable salt thereof is in suspended form. In some aspects, the suspension comprises a flocculating agent.

### Powder

In some aspects, the present disclosure provides a powder which is combined with a liquid to produce the pharmaceutical composition described herein.

In some aspects, the powder comprises losartan or a pharmaceutically acceptable form thereof. In some aspects, the powder comprises losartan. In some aspects, the powder comprises losartan potassium.

In some aspects, the powder comprises one or more pharmaceutically acceptable excipients.

In some aspects, the powder is bioequivalent to COZAAR®

In some aspects, the powder is sterile.

In some aspects, the powder is non-pyrogenic.

In some aspects, the powder is substantially free of impurities.

In some aspects, the liquid is a solvent. In some aspects, the solvent is water.

In some aspects, the liquid is provided in a container. In some aspects, the container is a bottle. In some aspects, the bottle is an amber bottle.

In some aspects, the container contains a suspending agent. In some aspects, the container contains a preservative. In some aspects, the container contains a sweetener. In some aspects, the container contains a flavoring agent.

In some aspects, the powder is packaged in a sachet.

### Granules

In some aspects, the present disclosure provides a plurality of granules which is combined with a liquid to produce the pharmaceutical composition described herein. In some aspects, the plurality of granules comprises losartan or a pharmaceutically acceptable salt thereof. In some aspects, the plurality of granules comprises losartan. In some aspects, the plurality of granules comprises losartan potassium.

In some aspects, the plurality of granules comprises one or more pharmaceutically acceptable excipients.

In some aspects, the plurality of granules is bioequivalent to COZAAR®.

In some aspects, the plurality of granules is sterile.

In some aspects, the plurality of granules is non-pyrogenic.

In some aspects, the plurality of granules is substantially free of impurities.

In some aspects, the liquid is a solvent. In some aspects, the solvent is water.

In some aspects, the liquid is provided in a container. In some aspects, the container is a bottle. In some aspects, the bottle is an amber bottle.

In some aspects, the container contains a suspending agent. In some aspects, the container contains a preservative. In some aspects, the container contains a sweetener. In some aspects, the container contains a flavoring agent.

In some aspects, the plurality of granules is packaged in a sachet.

### Particle Characterization

In some aspects, the suspension or the powder exhibits a dissolution profile as measured using a paddle type apparatus. In some aspects, the dissolution profile is measured at 50 rpm. In some aspects, the dissolution profile of the suspension or powder is measured in a mixture of water and 0.1N HCl. In some aspects, less than 35% of the plurality of losartan particles are dissolved in 15 minutes. In some aspects, less than 70% of the plurality of losartan particles are dissolved in 30 minutes. In some aspects, at least 80% of the plurality of losartan particles are dissolved in 60 minutes. In some aspects, at least 80% of the plurality of losartan particles are dissolved in 15 minutes. In some aspects, at least 85% of the plurality of losartan particles are dissolved in 15 minutes. In some aspects, at least 90% of the plurality of losartan particles are dissolved in 15 minutes. In some aspects, at least 95% of the plurality of losartan particles are dissolved in 15 minutes. In some aspects, about 100% of the plurality of losartan particles are dissolved in 15 minutes. In some aspects, at least 80% of the plurality of losartan particles are dissolved in 5 minutes. In some aspects, at least 85% of the plurality of losartan particles are dissolved in 5 minutes. In some aspects, at least 90% of the plurality of losartan particles are dissolved in 5 minutes. In some aspects, at least 95% of the plurality of losartan particles are dissolved in 5 minutes. In some aspects, about 100% of the plurality of losartan particles are dissolved in 5 minutes.

In some aspects, the suspension or powder exhibits a particle size distribution as measured using a particle size analyzer. In some aspects, the particle size analyzer is a Malvern Mastersizer particle size analyzer. In some aspects, the particle size analyzer is a Malvern Mastersizer 3000 particle size analyzer. In some aspects, the particle size analyzer is a Malvern Zetasizer particle size analyzer.

In some aspects, the particle size distribution comprises a D10 value less than 100 μm. In some aspects, the particle size distribution comprises a D50 value less than 500 μm. In some aspects, the particle size distribution comprises a D90 value less than 1000 μm. In some aspects, the particle size distribution comprises a D10 value less than 100 μm, a D50 value less than 500 μm, and a D90 value less than 1000 μm.

In some aspects, the particle size distribution comprises a D10 value less than 20 μm. In some aspects, the particle size distribution comprises a D50 value less than 100 μm. In some aspects, the particle size distribution comprises a D90 value less than 300 μm. In some aspects, the particle size distribution comprises a D10 value less than 20 μm, a D50 value less than 100 μm, and a D90 value less than 300 μm.

In some aspects, the particle size distribution comprises a D10 value less than 2 μm. In some aspects, the particle size distribution comprises a D50 value less than 10 μm. In some aspects, the particle size distribution comprises a D90 value less than 30 μm. In some aspects, the particle size distribution comprises a D10 value less than 2 μm, a D50 value less than 10 μm, and a D90 value less than 30 μm.

In some aspects, the particle size distribution comprises a D10 value less than 100 nm. In some aspects, the particle size distribution comprises a D50 value less than 500 nm. In some aspects, the particle size distribution comprises a D90 value less than 1000 nm. In some aspects, the particle size distribution comprises a D10 value less than 100 nm, a D50 value less than 500 nm, and a D90 value less than 1000 nm.

In some aspects, the particle size distribution comprises a D10 value less than or equal to 1 μm. In some aspects, the particle size distribution comprises a D50 value less than or equal to 5 μm. In some aspects, the particle size distribution comprises a D90 value less than or equal to 15 μm. In some aspects, the particle size distribution comprises a D10 value less than or equal to 1 μm, a D50 value less than or equal to 5 μm, and a D90 value less than or equal to 15 μm.

In some aspects, the particle size distribution comprises a D90 value less than 2 μm. In some aspects, the particle size distribution comprises a D90 value less than 1 μm. In some aspects, the particle size distribution comprises a D90 value less than 500 nm.

In some aspects, the suspension or the powder has a sedimentation rate of less than 10% over a period of 24 hours. In some aspects, the suspension or the powder has a sedimentation rate of less than 5% over a period of 24 hours.

### Modified Losartan

In some aspects, the pharmaceutical composition comprises a modified losartan. In some aspects, the powder comprises a modified losartan. In some aspects, the modified losartan comprises losartan or a pharmaceutically acceptable salt thereof complexed or coated with a wax, one or more polymers, or one or more other inactive ingredients. In some aspects, the modified losartan comprises losartan or a pharmaceutically acceptable salt thereof complexed or coated with a wax. In some aspects, the modified losartan comprises losartan or a pharmaceutically acceptable salt thereof complexed or coated with one or more polymers. In some aspects, the modified losartan is losartan or a pharmaceutically acceptable salt thereof coated with one or more inactive ingredients.

### Crystalline Form

In some aspects, the powder or suspension comprises losartan or a pharmaceutically acceptable salt thereof in crystalline form. In some aspects, the crystalline form is thermodynamically stable. In some aspects, the crystalline form is thermodynamically stable, as measured by a substantially unchanged X-ray powder diffraction (XRPD) profile following storage. In some aspects, the crystalline form is thermodynamically stable, as measured by a substantially unchanged differential scanning calorimetry (DSC) profile following storage.

### Concentration of Losartan

In some aspects, the pharmaceutical composition comprises about 1 mg/mL to about 50 mg/mL of losartan or pharmaceutically acceptable salt thereof. In some aspects, the pharmaceutical composition comprises about 2 mg/mL to about 20 mg/mL of losartan or a pharmaceutically acceptable salt thereof. In some aspects, the pharmaceutical composition comprises about 5 mg/mL to about 20 mg/mL of losartan or a pharmaceutically acceptable salt thereof. In some aspects, the pharmaceutical composition comprises about 8 mg/mL to about 12 mg/mL of losartan or a pharmaceutically acceptable salt thereof.

In some aspects, the pharmaceutical composition comprises about 1 mg/ml of losartan or a pharmaceutically acceptable salt thereof. In some aspects, the pharmaceutical composition comprises about 2 mg/mL of losartan or a pharmaceutically acceptable salt thereof. In some aspects, the pharmaceutical composition comprises about 3 mg/ml of losartan or a pharmaceutically acceptable salt thereof. In some aspects, the pharmaceutical composition comprises about 4 mg/mL of losartan or a pharmaceutically acceptable salt thereof. In some aspects, the pharmaceutical composition comprises about 5 mg/mL of losartan or a pharmaceutically acceptable salt thereof. In some aspects, the pharmaceutical composition comprises about 6 mg/mL of losartan or a pharmaceutically acceptable salt thereof. In some aspects, the pharmaceutical composition comprises about 7 mg/ml of losartan or a pharmaceutically acceptable salt thereof. In some aspects, the pharmaceutical composition comprises about 8 mg/mL of losartan or a pharmaceutically acceptable salt thereof. In some aspects, the pharmaceutical composition comprises about 9 mg/ml of losartan or a pharmaceutically acceptable salt thereof. In some aspects, the pharmaceutical composition comprises about 10 mg/mL of losartan or a pharmaceutically acceptable salt thereof. In some aspects, the pharmaceutical composition comprises about 11 mg/ml of losartan or a pharmaceutically acceptable salt thereof. In some aspects, the pharmaceutical composition comprises about 12 mg/mL of losartan or a pharmaceutically acceptable salt thereof. In some aspects, the pharmaceutical composition comprises about 13 mg/mL of losartan or a pharmaceutically acceptable salt thereof. In some aspects, the pharmaceutical composition comprises about 14 mg/mL of losartan or a pharmaceutically acceptable salt thereof. In some aspects, the pharmaceutical composition comprises about 15 mg/ml of losartan or a pharmaceutically acceptable salt thereof. In some aspects, the pharmaceutical composition comprises about 16 mg/mL of losartan or a pharmaceutically acceptable salt thereof. In some aspects, the pharmaceutical composition comprises about 17 mg/ml of losartan or a pharmaceutically acceptable salt thereof. In some aspects, the pharmaceutical composition comprises about 18 mg/mL of losartan or a pharmaceutically acceptable salt thereof. In some aspects, the pharmaceutical composition comprises about 19 mg/ml of losartan or a pharmaceutically acceptable salt thereof. In some aspects, the pharmaceutical composition comprises about 20 mg/mL of losartan or a pharmaceutically acceptable salt thereof. In some aspects, the pharmaceutical composition comprises about 21 mg/ml of losartan or a pharmaceutically acceptable salt thereof. In some aspects, the pharmaceutical composition comprises about 22 mg/mL of losartan or a pharmaceutically acceptable salt thereof. In some aspects, the pharmaceutical composition comprises about 23 mg/ml of losartan or a pharmaceutically acceptable salt thereof. In some aspects, the pharmaceutical composition comprises about 24 mg/mL of losartan or a pharmaceutically acceptable salt thereof. In some aspects, the pharmaceutical composition comprises about 25 mg/ml of losartan or a pharmaceutically acceptable salt thereof. In some aspects, the pharmaceutical composition comprises about 26 mg/mL of losartan or a pharmaceutically acceptable salt thereof. In some aspects, the pharmaceutical composition comprises about 27 mg/mL of losartan or a pharmaceutically acceptable salt thereof. In some aspects, the pharmaceutical composition comprises about 28 mg/mL of losartan or a pharmaceutically acceptable salt thereof. In some aspects, the pharmaceutical composition comprises about 29 mg/ml of losartan or a pharmaceutically acceptable salt thereof. In some aspects, the pharmaceutical composition comprises about 30 mg/mL of losartan or a pharmaceutically acceptable salt thereof. In some aspects, the pharmaceutical composition comprises about 31 mg/mL of losartan or a pharmaceutically acceptable salt thereof. In some aspects, the pharmaceutical composition comprises about 32 mg/mL of losartan or a pharmaceutically acceptable salt thereof. In some aspects, the pharmaceutical composition comprises about 33 mg/ml of losartan or a pharmaceutically acceptable salt thereof. In some aspects, the pharmaceutical composition comprises about 34 mg/mL of losartan or a pharmaceutically acceptable salt thereof. In some aspects, the pharmaceutical composition comprises about 35 mg/mL of losartan or a pharmaceutically acceptable salt thereof. In some aspects, the pharmaceutical composition comprises about 36 mg/mL of losartan or a pharmaceutically acceptable salt thereof. In some aspects, the pharmaceutical composition comprises about 37 mg/ml of losartan or a pharmaceutically acceptable salt thereof. In some aspects, the pharmaceutical composition comprises about 38 mg/mL of losartan or a pharmaceutically acceptable salt thereof. In some aspects, the pharmaceutical composition comprises about 39 mg/mL of losartan or a pharmaceutically acceptable salt thereof. In some aspects, the pharmaceutical composition comprises about 40 mg/mL of losartan or a pharmaceutically acceptable salt thereof. In some aspects, the pharmaceutical composition comprises about 41 mg/ml of losartan or a pharmaceutically acceptable salt thereof. In some aspects, the pharmaceutical composition comprises about 42 mg/mL of losartan or a pharmaceutically acceptable salt thereof. In some aspects, the pharmaceutical composition comprises about 43 mg/ml of losartan or a pharmaceutically acceptable salt thereof. In some aspects, the pharmaceutical composition comprises about 44 mg/mL of losartan or a pharmaceutically acceptable salt thereof. In some aspects, the pharmaceutical composition comprises about 45 mg/ml of losartan or a pharmaceutically acceptable salt thereof. In some aspects, the pharmaceutical composition comprises about 46 mg/mL of losartan or a pharmaceutically acceptable salt thereof. In some aspects, the pharmaceutical composition comprises about 47 mg/mL of losartan or a pharmaceutically acceptable salt thereof. In some aspects, the pharmaceutical composition comprises about 48 mg/mL of losartan or a pharmaceutically acceptable salt thereof. In some aspects, the pharmaceutical composition comprises about 49 mg/ml of losartan or a pharmaceutically acceptable salt thereof. In some aspects, the pharmaceutical composition comprises about 50 mg/mL of losartan or a pharmaceutically acceptable salt thereof.

pH

In some aspects, the pharmaceutical composition has a pH between 2 and 10. In some aspects, the pharmaceutical composition has a pH between 4 and 7. In some aspects, the pharmaceutical composition has a pH between 1 and 9. In some aspects, the pharmaceutical composition has a pH between 2 and 8. In some aspects, the pharmaceutical composition has a pH between 3 and 7. In some aspects, the pharmaceutical composition has a pH between 4 and 6. In some aspects, the pharmaceutical composition has a pH between 5 and 7. In some aspects, the pharmaceutical composition has a pH between 3 and 5. In some aspects, the pharmaceutical composition has a pH between 7 and 9. In some aspects, the pH is 4.2.

In some aspects, the pharmaceutical composition has a pH less than 10. In some aspects, the pharmaceutical composition has a pH less than 9. In some aspects, the pharmaceutical composition has a pH less than 8. In some aspects, the pharmaceutical composition has a pH less than 7. In some aspects, the pharmaceutical composition has a pH less than 6. In some aspects, the pharmaceutical composition has a pH less than 5. In some aspects, the pharmaceutical composition has a pH less than 4. In some aspects, the pharmaceutical composition has a pH less than 3. In some aspects, the pharmaceutical composition has a pH less than 2.

In some aspects, the pharmaceutical composition has a pH greater than 10. In some aspects, the pharmaceutical composition has a pH greater than 9. In some aspects, the pharmaceutical composition has a pH greater than 8. In some aspects, the pharmaceutical composition has a pH greater than 7. In some aspects, the pharmaceutical composition has a pH greater than 6. In some aspects, the pharmaceutical composition has a pH greater than 5. In some aspects, the pharmaceutical composition has a pH greater than 4. In some aspects, the pharmaceutical composition has a pH greater than 3. In some aspects, the pharmaceutical composition has a pH greater than 2.

In some aspects, the pharmaceutical composition has a pH of about 2. In some aspects, the pharmaceutical composition has a pH of about 2.5. In some aspects, the pharmaceutical composition has a pH of about 3. In some aspects, the pharmaceutical composition has a pH of about 3.5. In some aspects, the pharmaceutical composition has a pH of about 4. In some aspects, the pharmaceutical composition has a pH of about 4.1. In some aspects, the pharmaceutical composition has a pH of about 4.2. In some aspects, the pharmaceutical composition has a pH of about 4.3. In some aspects, the pharmaceutical composition has a pH of about 4.4. In some aspects, the pharmaceutical composition has a pH of about 4.5. In some aspects, the pharmaceutical composition has a pH of about 5. In some aspects, the pharmaceutical composition has a pH of about 5.5. In some aspects, the pharmaceutical composition has a pH of about 6. In some aspects, the pharmaceutical composition has a pH of about 6.5. In some aspects, the pharmaceutical composition has a pH of about 7. In some aspects, the pharmaceutical composition has a pH of about 7.5. In some aspects, the pharmaceutical composition has a pH of about 8. In some aspects, the pharmaceutical composition has a pH of about 8.5. In some aspects, the pharmaceutical composition has a pH of about 9. In some aspects, the pharmaceutical composition has a pH of about 9.5. In some aspects, the pharmaceutical composition has a pH of about 10.

In some aspects, the pharmaceutical composition has a pH of 2. In some aspects, the pharmaceutical composition has a pH of 2.5. In some aspects, the pharmaceutical composition has a pH of 3. In some aspects, the pharmaceutical composition has a pH of 3.5. In some aspects, the pharmaceutical composition has a pH of 4. In some aspects, the pharmaceutical composition has a pH of 4.1. In some aspects, the pharmaceutical composition has a pH of 4.2. In some aspects, the pharmaceutical composition has a pH of 4.3. In some aspects, the pharmaceutical composition has a pH of 4.4. In some aspects, the pharmaceutical composition has a pH of 4.5. In some aspects, the pharmaceutical composition has a pH of 5. In some aspects, the pharmaceutical composition has a pH of 5.5. In some aspects, the pharmaceutical composition has a pH of 6. In some aspects, the pharmaceutical composition has a pH of 6.5. In some aspects, the pharmaceutical composition has a pH of 7. In some aspects, the pharmaceutical composition has a pH of 7.5. In some aspects, the pharmaceutical composition has a pH of 8. In some aspects, the pharmaceutical composition has a pH of 8.5. In some aspects, the pharmaceutical composition has a pH of 9. In some aspects, the pharmaceutical composition has a pH of 9.5. In some aspects, the pharmaceutical composition has a pH of 10.

### Pharmacokinetic Parameters and Bioequivalence

In some aspects, the mean Tₘₐₓ of the pharmaceutical composition described herein is about 98% to about 102% of the mean Tₘₐₓ of COZAAR®.

In some aspects, the mean Tₘₐₓ of the pharmaceutical composition described herein is about 95% to about 105% of the mean Tₘₐₓ of COZAAR®.

In some aspects, the mean Tₘₐₓ of the pharmaceutical composition described herein is about 90% to about 110% of the mean Tₘₐₓ of COZAAR®.

In some aspects, the mean Tₘₐₓ of the pharmaceutical composition described herein is about 80% to about 125% of the mean Tₘₐₓ of COZAAR®.

In some aspects, the mean Tₘₐₓ of the pharmaceutical composition described herein is about 75% to about 130% of the mean Tₘₐₓ of COZAAR®.

In some aspects, the mean Tₘₐₓ of the pharmaceutical composition described herein is about 70% to about 135% of the mean Tₘₐₓ of COZAAR®.

In some aspects, the mean Tₘₐₓ of the pharmaceutical composition described herein is about 65% to about 140% of the mean Tₘₐₓ of COZAAR®.

In some aspects, the mean Tₘₐₓ of the pharmaceutical composition described herein is about 60% to about 145% of the mean Tₘₐₓ of COZAAR®.

In some aspects, the mean Tₘₐₓ of the pharmaceutical composition described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the mean Tₘₐₓ of COZAAR®.

In some aspects, the mean Tₘₐₓ of the pharmaceutical composition described herein is about 50% to about 150% of the mean Tₘₐₓ of COZAAR®. In some aspects, the mean Tₘₐₓ of the pharmaceutical composition described herein is about 40% to about 160% of the mean Tₘₐₓ of COZAAR®. In some aspects, the mean Tₘₐₓ of the pharmaceutical composition described herein is about 30% to about 170% of the mean Tₘₐₓ of COZAAR®. In some aspects, the mean Tₘₐₓ of the pharmaceutical composition described herein is about 20% to about 200% of the mean Tₘₐₓ of COZAAR®.

In some aspects, the mean Cₘₐₓ of the pharmaceutical composition described herein is about 98% to about 102% of the mean Cₘₐₓ of COZAAR®.

In some aspects, the mean Cₘₐₓ of the pharmaceutical composition described herein is about 95% to about 105% of the mean Cₘₐₓ of COZAAR®.

In some aspects, the mean Cₘₐₓ of the pharmaceutical composition described herein is about 90% to about 110% of the mean Cₘₐₓ of COZAAR®.

In some aspects, the mean Cₘₐₓ of the pharmaceutical composition described herein is about 80% to about 125% of the mean Cₘₐₓ of COZAAR®.

In some aspects, the mean Cₘₐₓ of the pharmaceutical composition described herein is about 75% to about 130% of the mean Cₘₐₓ of COZAAR®.

In some aspects, the mean Cₘₐₓ of the pharmaceutical composition described herein is about 70% to about 135% of the mean Cₘₐₓ of COZAAR®.

In some aspects, the mean Cₘₐₓ of the pharmaceutical composition described herein is about 65% to about 140% of the mean Cₘₐₓ of COZAAR®.

In some aspects, the mean Cₘₐₓ of the pharmaceutical composition described herein is about 60% to about 145% of the mean Cₘₐₓ of COZAAR®.

In some aspects, the mean Cₘₐₓ of the pharmaceutical composition described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the mean Cₘₐₓ of COZAAR®.

In some aspects, the mean AUC_{0-∞} of the pharmaceutical composition described herein is about 98% to about 102% of the mean AUC_{0-∞} of COZAAR®.

In some aspects, the mean AUC_{0-∞} of the pharmaceutical composition described herein is about 95% to about 105% of the mean AUC_{0-∞} of COZAAR®.

In some aspects, the mean AUC_{0-∞} of the pharmaceutical composition described herein is about 90% to about 110% of the mean AUC_{0-∞} of COZAAR®.

In some aspects, the mean AUC_{0-∞} of the pharmaceutical composition described herein is about 80% to about 125% of the mean AUC_{0-∞} of COZAAR®.

In some aspects, the mean AUC_{0-∞} of the pharmaceutical composition described herein is about 75% to about 130% of the mean AUC_{0-∞} of COZAAR®.

In some aspects, the mean AUC_{0-∞} of the pharmaceutical composition described herein is about 70% to about 135% of the mean AUC_{0-∞} of COZAAR®.

In some aspects, the mean AUC_{0-∞} of the pharmaceutical composition described herein is about 65% to about 140% of the mean AUC_{0-∞} of COZAAR®.

In some aspects, the mean AUC_{0-∞} of the pharmaceutical composition described herein is about 60% to about 145% of the mean AUC_{0-∞} of COZAAR®.

In some aspects, the mean AUC_{0-∞} of the pharmaceutical composition described herein is about 60%, about 65%, about 70%, about 75%, about 80%, about 85%, about 90%, about 95%, about 100%, about 105%, about 110%, about 115%, about 120% about 125%, about 130%, about 135%, about 140%, or about 145% of the mean AUC_{0-∞} of COZAAR®

### Additional Active Ingredients

In some aspects, the pharmaceutical composition comprises one or more active ingredients in addition to losartan or a pharmaceutically acceptable salt thereof. In some aspects, the one or more active ingredients are selected from the group consisting of: calcium channel blockers, diuretics, ACE inhibitors, and beta blockers. In some aspects, the one or more active ingredients are calcium channel blockers. In some aspects, the one or more active ingredients are diuretics. In some aspects, the one or more active ingredients are ACE inhibitors. In some aspects, the one or more active ingredients are beta blockers.

### Viscosity

In some aspects, the pharmaceutical composition has a viscosity of less than 2000 centipoise, as measured using a Brookfield viscometer. In some aspects, the pharmaceutical composition has a viscosity of less than 1000 centipoise, as measured using a Brookfield viscometer. In some aspects, the pharmaceutical composition has a viscosity of about 100 to 2000 centipoise, as measured using a Brookfield viscometer. In some aspects, the pharmaceutical composition has a viscosity of about 100 to 1500 centipoise, as measured using a Brookfield viscometer. In some aspects, the pharmaceutical composition has a viscosity of about 100 to 1000 centipoise, as measured using a Brookfield viscometer. In some aspects, the pharmaceutical composition has a viscosity of about 500 to 2000 centipoise, as measured using a Brookfield viscometer. In some aspects, the pharmaceutical composition has a viscosity of about 100 to 2500 centipoise, as measured using a Brookfield viscometer.

### Volume

In some aspects, the pharmaceutical composition is administered in a volume of 0.5 mL to 50 mL. In some aspects, the pharmaceutical composition is administered in a volume of less than 100 mL. In some aspects, the pharmaceutical composition is administered in a volume of less than 90 mL. In some aspects, the pharmaceutical composition is administered in a volume of less than 80 mL. In some aspects, the pharmaceutical composition is administered in a volume of less than 70 mL. In some aspects, the pharmaceutical composition is administered in a volume of less than 60 mL. In some aspects, the pharmaceutical composition is administered in a volume of less than 50 mL. In some aspects, the pharmaceutical composition is administered in a volume of less than 40 mL. In some aspects, the pharmaceutical composition is administered in a volume of less than 30 mL. In some aspects, the pharmaceutical composition is administered in a volume of less than 20 mL. In some aspects, the pharmaceutical composition is administered in a volume of less than 10 mL. In some aspects, the pharmaceutical composition is administered in a volume of less than 5 mL.

In some aspects, the pharmaceutical composition is administered in a volume of about 0.1 mL, about 0.2 mL, about 0.3 mL, about 0.4 mL, about 0.5 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.1 mL, about 2.2 mL, about 2.3 mL, about 2.4 mL, about 2.5 mL, about 3 mL, about 3.5 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL, or about 10 mL.

In some aspects, the pharmaceutical composition is administered in a volume of about 0.1 mL to about 20 mL, about 0.1 mL to about 15 mL, about 0.1 mL to about 10 mL, about 0.1 mL to about 2.5 mL, about 0.5 mL to about 2.5 mL, about 1 mL to about 10 mL, about 1 mL to about 2.5 mL, about 2 mL to about 10 mL, about 2 mL to about 3 mL, about 4 mL to about 6 mL, or about 9 mL to about 11 mL. In some aspects, the pharmaceutical composition is administered in a volume of about 2.5 mL to about 10 mL.

### Container

In some aspects, a pharmaceutical composition is packaged in a glass container. In some aspects, the powder is packaged in a glass container. In some aspects, the glass container is an amber glass container. In some aspects, the amber glass container is child resistant. In some aspects, the amber glass container comprises a child resistant cap. In some aspects, the pharmaceutical composition is packaged in a polymeric container. In some aspects, the polymeric container is child resistant. In some aspects, the polymeric container comprises a child resistant cap. In some aspects, the polymeric container comprises high density polyethylene (HDPE). In some aspects, the polymeric container comprises low density polyethylene (LDPE).

### Kit

In some aspects, the present disclosure provides for a kit comprising a pharmaceutically acceptable composition described herein, and further comprising a set of instructions for administration of the pharmaceutically acceptable composition to a subject in need thereof. In some aspects, the set of instructions comprises an instruction to add an amount of a diluent to a container comprising a concentrated form of losartan or a pharmaceutically acceptable salt thereof, wherein the kit comprises the container. In some aspects, the diluent is water.

In some aspects, the present disclosure provides for a kit comprising: a concentrated form of losartan or a pharmaceutically acceptable salt thereof; a diluent; and a set of instructions, wherein the set of instructions comprises: instructions for combining the concentrated form of losartan or a pharmaceutically acceptable salt thereof and the diluent to form a pharmaceutically acceptable composition described herein; and instructions for administration of the pharmaceutically acceptable composition to a subject in need thereof. In some aspects, the concentrated form of losartan is in the form of a powder or a plurality of granules. In some aspects, the concentrated form of losartan or a pharmaceutically acceptable salt thereof is in the form of a powder. In some aspects, the concentrated form of losartan or a pharmaceutically acceptable salt thereof is in the form of a plurality of granules.

In some aspects, the kit comprises a concentrated form of a pharmaceutical composition disclosed herein. In some aspects, the concentrated form is prepared by combining components of a pharmaceutical composition disclosed herein without dilution to a final concentration suitable for administration to a subject. In some aspects, the concentrated form is a concentrated liquid form. In some aspects, the concentrated liquid form is a solution. In some aspects, the concentrated liquid form is a suspension. In some aspects, the concentrated form is a dry form. In some aspects, the dry form comprises less than 0.1%, 0.5%, 1%, 2%, 3%, 4%, 5%, 6%, 7%, 8%, 9%, or 10% of water by weight of the dry form. In some aspects, the dry form is a powder or a plurality of granules.

### Method of Preparing Suspension

In some aspects, the present disclosure provides a method of preparing a suspension described herein, the method comprising combining a plurality of losartan particles and a suspending agent. In some aspects, the plurality of losartan particles is combined with the suspending agent.

In some aspects, the present disclosure provides a method of preparing a suspension described herein, the method comprising dissolving losartan or a pharmaceutically acceptable salt thereof in a suitable fluid (e.g., water), adjusting the pH of the suitable liquid comprising losartan or a pharmaceutically acceptable salt thereof, and combining the suitable liquid comprising losartan or a pharmaceutically acceptable salt thereof with the remaining components of the suspension, thereby forming the suspension.

In some aspects, the present disclosure provides a method of preparing a suspension described herein, the method comprising combining losartan or a pharmaceutically acceptable salt thereof and one or more excipients, granulating the composition with a suitable fluid (e.g., water), drying the resulting composition to form a dry composition, and combining the dry composition with a suitable liquid, thereby forming the suspension.

In some aspects, the present disclosure provides a method of preparing a suspension described herein, the method comprising a step of particle size reduction. In some aspects, the step of particle size reduction comprises wet milling. In some aspects, the step of particle size reduction comprises microfluidation. In some aspects, the step of particle size reduction comprises homogenization. In some aspects, the step of particle size reduction comprises nano-milling.

In some aspects, a pH modifying agent is added after the plurality of losartan particles is combined with the suspending agent. In some aspects, the pH modifying agent is selected from the group consisting of: citric acid, sodium citrate, acetic acid, sodium acetate, sodium hydroxide, dibasic calcium phosphate, sodium dihydrogen phosphate, and disodium hydrogen phosphate. In some aspects, the pH modifying agent is a buffer.

In some aspects, the one or more polymers are sprayed by dissolving the one or more polymers in a volatile solvent. In some aspects, the volatile solvent is selected from the group consisting of: ethanol, acetone, or isopropyl alcohol. In some aspects, the volatile solvent is ethanol. In some aspects, the volatile solvent is acetone. In some aspects, the volatile solvent is isopropyl alcohol.

In some aspects, heated air is added to cause evaporation of the volatile solvent. In some aspects, evaporation of the volatile solvent causes deposition of the one or more polymers onto the surface of a particle comprising losartan or a pharmaceutically acceptable salt thereof. In some aspects, the particles comprising losartan or a pharmaceutically acceptable salt thereof are dried to form a modified losartan. In some aspects, the plurality of losartan particles is provided as a modified losartan which is preformed. In some aspects, the plurality of losartan particles comprises losartan or a pharmaceutically acceptable salt thereof.

In some aspects, the plurality of losartan particles are fluidized in a fluidized bed system, and are then coated with one or more polymers. In some aspects, the plurality of losartan particles comprises losartan or a pharmaceutically acceptable salt thereof. In some aspects, the plurality of losartan particles has a particle size of less than 1000 μm. In some aspects, the plurality of losartan particles has a particle size of less than 950 μm. In some aspects, the plurality of losartan particles has a particle size of less than 900 μm. In some aspects, the plurality of losartan particles has a particle size of less than 850 μm. In some aspects, the plurality of losartan particles has a particle size of less than 800 μm. In some aspects, the plurality of losartan particles has a particle size of less than 750 μm. In some aspects, the plurality of losartan particles has a particle size of less than 700 μm. In some aspects, the plurality of losartan particles has a particle size of less than 650 μm. In some aspects, the plurality of losartan particles has a particle size of less than 600 μm. In some aspects, the plurality of losartan particles has a particle size of less than 550 μm. In some aspects, the plurality of losartan particles has a particle size of less than 500 μm. In some aspects, the plurality of losartan particles has a particle size of less than 450 μm. In some aspects, the plurality of losartan particles has a particle size of less than 400 μm. In some aspects, the plurality of losartan particles has a particle size of less than 350 μm. In some aspects, the plurality of losartan particles has a particle size of less than 300 μm. In some aspects, the plurality of losartan particles has a particle size of less than 250 μm. In some aspects, the plurality of losartan particles has a particle size of less than 200 μm. In some aspects, the plurality of losartan particles has a particle size of less than 150 μm. In some aspects, the plurality of losartan particles has a particle size of less than 100 μm. In some aspects, the plurality of losartan particles has a particle size of less than 50 μm.

In some aspects, the one or more polymers comprise pH dependent polymers (e.g., Eudragit L, Eudragit S, or cellulose acetate phthalate) and/or pH independent polymers (e.g., Eudragit RS, Eudragit RL, or cellulose acetate). In some aspects, the one or more polymers comprise pH dependent polymers (e.g., Eudragit L, Eudragit S, or cellulose acetate phthalate). In some aspects, the one or more polymers comprise Eudragit L. In some aspects, the one or more polymers comprise Eudragit S. In some aspects, the one or more polymers comprise cellulose acetate phthalate. In some aspects, the one or more polymers comprise pH independent polymers (e.g., Eudragit RS, Eudragit RL, or cellulose acetate). In some aspects, the one or more polymers comprise Eudragit RS. In some aspects, the one or more polymers comprise Eudragit RL. In some aspects, the one or more polymers comprise cellulose acetate.

In some aspects, the modified losartan tastes less bitter than losartan.

In some aspects, the modified losartan is more stable than losartan.

In some aspects, a composition of the present disclosure is diluted prior to administration. In some aspects, the composition is diluted with a diluent. In some aspects, the diluent is a composition disclosed herein, but excludes losartan (i.e., contains the same components and concentrations as a composition disclosed herein, with the exception that losartan or a pharmaceutically acceptable salt thereof is not included in the diluent).

In some aspects, a diluted composition disclosed herein is for administration to a pediatric subject. In some aspects, a diluted composition disclosed herein is for administration to a neonatal subject. In some aspects, a diluted composition disclosed herein is for administration to a low weight subject. In some aspects, a diluted composition disclosed herein is for administration to a non-human animal.

In some aspects, the composition is diluted to a target concentration. In some aspects, the target concentration is about 0.1 mg/mL losartan or a pharmaceutically acceptable salt thereof. In some aspects, the target concentration is about 0.2 mg/mL losartan or a pharmaceutically acceptable salt thereof. In some aspects, the target concentration is about 0.3 mg/mL losartan or a pharmaceutically acceptable salt thereof. In some aspects, the target concentration is about 0.4 mg/mL losartan or a pharmaceutically acceptable salt thereof. In some aspects, the target concentration is about 0.5 mg/ml losartan or a pharmaceutically acceptable salt thereof. In some aspects, the target concentration is about 0.6 mg/mL losartan or a pharmaceutically acceptable salt thereof. In some aspects, the target concentration is about 0.7 mg/mL losartan or a pharmaceutically acceptable salt thereof. In some aspects, the target concentration is about 0.8 mg/mL losartan or a pharmaceutically acceptable salt thereof. In some aspects, the target concentration is 0.9 mg/mL losartan or a pharmaceutically acceptable salt thereof. In some aspects, the target concentration is about 1.0 mg/mL losartan or a pharmaceutically acceptable salt thereof.

### Method of Preparing Solution or Emulsion

In some aspects, the present disclosure provides a method of preparing a solution or an emulsion described herein, comprising combining the components of the solution or the emulsion.

In some aspects, losartan or a pharmaceutically acceptable salt thereof is dissolved in a hydrophobic phase (e.g., vegetable oil). In some aspects, an antioxidant is added to a hydrophobic phase. In some aspects, the hydrophobic phase comprising losartan or a pharmaceutically acceptable salt thereof is mixed with a hydrophilic phase. In some aspects, the hydrophilic phase comprises a sweetener. In some aspects, the hydrophilic phase comprises a flavoring agent. In some aspects, the hydrophilic phase comprises Polysorbate 80 and/or Tweens. In some aspects, the emulsion is prepared by combining and mixing the hydrophobic phase and the hydrophilic phase. In some aspects, the hydrophobic phase and hydrophilic phase are mixed at a high speed using a homogenizer.

### Method of Preparing Powder

In some aspects, the present disclosure provides a method of preparing a powder, the method comprising dry blending the components of a pharmaceutical composition described herein. In some aspects, the pharmaceutical composition is a suspension described herein.

### Method of Preparing Modified Losartan

In some aspects, the present disclosure provides a method of preparing modified losartan, the method comprising complexing or coating losartan or a pharmaceutically acceptable salt thereof with a substance, wherein the substance is a wax, a polymer, or one or more other inactive ingredients. In some aspects, the modified losartan is formed during preparation of the pharmaceutical composition. In some aspects, modified losartan is formed by complexation with an ion exchange resin. In some aspects, modified losartan is formed by complexation with a cyclodextrin. In some aspects, the modified losartan is prepared by microencapsulation of losartan or a pharmaceutically acceptable salt thereof in a coat of inactive ingredients.

### Method of Treatment

In some aspects, the present disclosure provides a method of treating a subject in need thereof comprising administering to the subject a therapeutically effective amount of the pharmaceutical composition described herein.

In some aspects, the present disclosure provides a method of treating hypertension in a subject in need thereof comprising administering to the subject a therapeutically effective amount of the pharmaceutical composition described herein. In some aspects, the present disclosure provides a method of treating adult hypertension in a subject in need thereof comprising administering to the subject a therapeutically effective amount of the pharmaceutical composition described herein. In some aspects, the present disclosure provides a method of treating pediatric hypertension in a subject in need thereof comprising administering to the subject a therapeutically effective amount of the pharmaceutical composition described herein. In some aspects, the subject is a child greater than 6 years old. In some aspects, the present disclosure provides a method of treating hypertension in a subject in need thereof comprising administering to the subject a therapeutically effective amount of the pharmaceutical composition described herein wherein the subject is a patient with left ventricular hypertrophy. In some aspects, the present disclosure provides a method of treating nephropathy in a subject in need thereof comprising administering to the subject a therapeutically effective amount of the pharmaceutical composition described herein. In some aspects, the present disclosure provides a method of treating nephropathy in a subject in need thereof comprising administering to the subject a therapeutically effective amount of the pharmaceutical composition described herein wherein the subject is a patient with type 2 diabetes.

In some aspects, the subject has been diagnosed with pre-hypertension or hypertension. In some aspects, the subject has been diagnosed with pre-hypertension. In some aspects, the subject has been diagnosed with hypertension. In some aspects, the subject has a history of hypertension.

In some aspects, the subject has been diagnosed with left ventricular hypertrophy. In some aspects, the subject has been diagnosed with both hypertension and left ventricular hypertrophy.

In some aspects, the subject has been diagnosed with type 2 diabetes.

In some aspects, the subject has been diagnosed with nephropathy.

In some aspects, the subject has been diagnosed with diabetic nephropathy.

In some aspects, the subject has been diagnosed with proteinuria.

In some aspects, the subject exhibits a urinary albumin to creatinine ratio of greater than or equal to 300 mg/g.

In some aspects, the subject has been diagnosed with an elevated serum creatinine.

In some aspects, the present disclosure provides a method of lowering blood pressure, reducing the risk of fatal and/or nonfatal cardiovascular events, primarily strokes, and/or myocardial infarctions in a subject in need thereof, comprising administering a pharmaceutical composition disclosed herein. In some aspects, the subject has hypertension and left ventricular hypertrophy. In some aspects, the present disclosure provides a method of treating diabetic nephropathy with an elevated serum creatinine and/or proteinuria (e.g., urinary albumin to creatinine ratio≥300 mg/g) in a subject in need thereof. In some aspects, the subject has type 2 diabetes and/or a history of hypertension.

In some aspects, the pharmaceutical composition is administered for: the treatment of hypertension, and to lower blood pressure in adults and children greater than 6 years old; to reduce of the risk of stroke in patients with hypertension and left ventricular hypertrophy; and to treat diabetic nephropathy with an elevated serum creatinine and proteinuria in patients with type 2 diabetes and a history of hypertension.

In some aspects, the subject is not a Black subject.

In some aspects, the pharmaceutical composition is co-administered with at least one other pharmaceutical agent. In some aspects, the at least one pharmaceutical agent is an antihypertensive agent. In some aspects, the antihypertensive agent is selected from the group consisting of: an angiotensin II antagonist, an angiotensin converting enzyme inhibitor, or a neutral endopeptidase/angiotensin converting enzyme inhibitor. In some aspects, the antihypertensive agent is an angiotensin II antagonist. In some aspects, the antihypertensive agent is an angiotensin converting enzyme inhibitor. In some aspects, the antihypertensive agent is a neutral endopeptidase/angiotensin converting enzyme inhibitor.

In some aspects, the method for treating hypertension to lower blood pressure in an adult or pediatric subject greater than 6 years old, comprises administering an oral suspension comprising 10 mg/mL losartan potassium.

In some aspects, the method for reducing the risk of stroke in a subject with hypertension and left ventricular hypertrophy comprises administering an oral suspension comprising 10 mg/mL losartan potassium.

In some aspects, the method for treating diabetic nephropathy with an elevated serum creatinine and proteinuria in a subject with type 2 diabetes and a history of hypertension comprises administering an oral suspension comprising 10 mg/mL losartan potassium.

In some aspects, the oral suspension comprises losartan potassium at 10 mg/mL, dibasic sodium phosphate, hypromellose, methyl paraben, monobasic sodium phosphate, natural peppermint flavor, polyethylene glycol, povidone, propyl paraben, propylene glycol, purified water, simethicone, sucralose, and xanthan gum.

In some aspects, the starting dose is 50 mg orally once daily.

In some aspects, the maximum daily oral dose is 100 mg.

In some aspects, the starting dose for patients with possible intravascular depletion is 25 mg.

In some aspects, the starting dose for a pediatric subject is 0.7 mg per kg.

In some aspects, the hydrochlorothiazide is co-administered at 12.5 mg daily.

In some aspects, the hydrochlorothiazide is administered at 25 mg once daily.

In some aspects, the dose is increased to 100 mg orally once daily based on blood pressure response.

In some aspects, the starting dose is 25 mg orally once daily for a subject with mild-to-moderate hepatic impairment.

In some aspects, the subject is not pregnant and/or not a breastfeeding woman.

In some aspects, the oral suspension is not co-administered with

(a) drugs or supplements that raise serum potassium levels,

(b) drugs that increase serum lithium concentrations,

(c) NSAIDs

(d) ACE inhibitors

(e) drugs that act as renin-angiotensin system (RAS) and angiotensin receptor blockers

(f) aliskiren

(g) agents that affect the renin-angiotensin system (RAS)

(h) inhibitors of cytochrome P450.

In some aspects, the adult subject is 65 years and over.

In some aspects, the pediatric subject is age 6 to 16.

In some aspects, the subject is not a black subject.

In some aspects, the subject has renal impairment.

Having described the invention with reference to the different aspects of the invention, other aspects will become apparent to one skilled in the art from consideration of the specification.

The innovation is further defined by reference to the following examples. It will be apparent to those skilled in the art that many modifications to the composition may be practiced without departing from the scope of this invention.

### EXAMPLES

In non-limiting examples, pharmaceutical compositions of losartan, methods of producing the same, and therapeutic uses thereof are disclosed herein. Examples 1, 2, 6, and 13 and the Tables 1, 2, 5, and 12 therein shows alternatives for formulation of an oral suspension of losartan. Example 3 provides a method of producing the oral suspension of Example 2, and Example 14 provides a method of producing the suspension of Example 13. Examples 4, 5, and 10-12, and Tables 3, 4, and 9-11 therein provide oral liquid solutions of losartan. Examples 7-9 and Tables 6-8 therein provide powder compositions suitable for reconstitution to prepare a solution (Example 7 and 8) or a suspension (Example 9). Examples 10-13 and Tables 9-12 therein provide alternatives for losartan solution for oral administration. Example 14 provides a method of producing a suspension of Example 13. Example 15 and Tables 13 and 14 therein show an analysis of losartan formulations administered to rabbits. Example 16 and Tables 15 show the dissolution, impurity, and stability analysis for the formulation of Group 4. Example 17 and Table 16 therein show the analysis of the formulation of Group 5 under various conditions. Example 18 and Table 17 show the impurity and stability analysis for the formulation of Example 5. Example 19 provides an exemplary formulation of the disclosure. Example 20 provides the supply, storage and handling guidelines. Example 21 discloses contraindications. Example 22 discloses warnings and precautions. Example 23 and Table 18 therein disclose adverse reactions. Example 24 describes postmarketing experience. Example 25 discloses drug interactions. Example 26 discloses use in specific populations. Example 27 discloses overdosage. Example 28 and Table 19 disclose clinical pharmacology and pharmacokinetic parameters in hypertensive adults and children age 6 to 16 following multiple dosing. Example 29 discloses non-clinical toxicology. Example 30 and Table 20 therein disclose clinical study on hypertension and hypertensive patients with left ventricular hypertrophy and the results for the primary composite endpoint and the individual endpoints. Example 31 and Tables 21-22 disclose clinical study on nephropathy in type 2 diabetic patients, incidence of primary endpoint events (Table 21) and efficacy outcomes within demographic subgroups (Table 22).

### Example 1: Suspension 1

A suspension of losartan suitable for oral administration was prepared having ingredients in the following proportions:

TABLE 1
Losartan Suspension for Oral Administration
Losartan Potassium Oral Suspension, 10 mg/mL

| INGREDIENT | Composition Qty/100 mL | |
|---|---|---|
| purified water, USP | 80-100 | g |
| methylparaben, NF | 0.1-0.3 | g |
| propylparaben, NF | 0.01-0.03 | g |
| losartan potassium, USP | 0.5-1.5 | g |
| hydroxy ethyl cellulose | 1-1.4 | g |
| (NATROSOL ™ 250 L), NF | | |
| Xanthan gum, NF (XANTURAL ® 180) | 0.5-1.5 | g |
| microcrystalline cellulose and | 1-2 | g |
| carboxymethylcellulose sodium, NF | | |
| (AVICEL ® RC-591) | | |
| disodium edetate, NF | 0.1-0.2 | g |
| simethicone emulsion 30%, USP (Q7- | 0.8-1.2 | g |
| 2587 simethicone emulsion 30%, USP) | | |
| a flavoring agent | 5-15 | mg |
| sucralose micronized, NF (SPLENDA ®) | 0.2-0.6 | g |

| | |
|---|---|
| anhydrous citric acid, USP | Qs to desired pH (5% |
| | citric acid solution |
| | in purified water) |

### Example 2: Suspension 2

A suspension of losartan suitable for oral administration was prepared having ingredients in the following proportions:

TABLE 2
Losartan Suspension for Oral Administration
Losartan Potassium Oral Suspension, 10 mg/mL

| | | Composition | | | |
|---|---|---|---|---|---|
| ITEM | | Qty/100 mL | | | |
| NO. | INGREDIENT | pH-4.2 | | pH-5.0 | |
| 1 | purified water, USP | 80-100 | g | 80-100 | g |
| 2 | methylparaben, NF | 0.1-0.3 | g | 0.18 | g |
| 3 | propylparaben, NF | 0.02 | g | 0.02 | g |
| 4 | losartan potassium, USP | 1.000 | g | 1.000 | g |
| 5 | hydroxyethyl cellulose | 1.20 | g | 1.20 | g |
| | (NATROSOL ™ 250 L), NF | | | | |
| 6 | Xanthan gum, NF (XANTURAL ® 180) | 0.10 | g | 0.10 | g |
| 7 | microcrystalline cellulose and | 1.50 | g | 1.50 | g |
| | carboxymethylcellulose sodium, NF | | | | |
| | (AVICEL ® RC-591) | | | | |
| 8 | disodium edetate, NF | 0.15 | g | 0.15 | g |
| 9 | simethicone emulsion 30%, USP | 1.00 | g | 1.00 | g |
| | (Q7-2587 simethicone emulsion 30%, USP) | | | | |
| 10 | sucralose micronized, NF (SPLENDA ®) | 0.40 | g | 0.40 | g |

| | | |
|---|---|---|
| 11 | anhydrous citric acid, USP | Qs to adjust pH (5% |
| | | citric acid solution |
| | | with purified water) |
| 12 | flavoring agent | 5-15 mg |

### Example 3: Method of Producing Suspension of Example 2

1. Preservatives (methylparaben, NF and propylparaben, NF) were dissolved in purified water at a temperature 80-85° C. Once dissolved, the content was cooled to 25° C.

2. Losartan (losartan potassium, USP) was added to the above solution once the temperature was between 25-30° C. and the drug was dissolved in water with preservatives.

3. A 5% citric acid solution was added with continuous homogenizing in an amount such that the final pH of the composition after all components were added would be the desired pH (here, either 4.2 or 5.0).

4. The viscosity modifying agents/thickening agents/suspending agents (hydroxyethyl cellulose (NATROSOL™ 250 L), NF and xanthan gum, NF (XANTURAL® 180)) were added to the composition of step 2 using high shear mixing.

5. Disodium edetate and simethicone emulsion 30% were added to the composition of step 4.

6. Sweetener was added and the composition was mixed for a few minutes.

7. A flavoring agent was added.

8. Qs to batch volume with purified water and composition was mixed.

In some aspects, the pH modifying agent (here, citric acid in step 3) is added after any other step.

### Example 4: Losartan Solution

A solution of losartan suitable for oral administration was prepared having ingredients in the following proportions:

TABLE 3
Losartan Solution for Oral Administration
Losartan Potassium Oral Solution, 10 mg/mL

| ITEM | | Composition | |
|---|---|---|---|
| NO. | INGREDIENTS | Qty/100 mL | |
| 1 | purified water, USP | 90.00 g | 90.00 g |
| 2 | methylparaben, NF | 0.18 g | 0.18 g |
| 3 | propylparaben, NF | 0.02 g | 0.02 g |
| 4 | losartan potassium, USP | 1.000 g | 1.000 g |
| 5 | hydroxyethyl cellulose | 1.20 g | 1.20 g |
| | (NATROSOL ™ 250 L), NF | | |
| 8 | disodium edetate, NF | 0.15 g | 0.15 g |
| 10 | sucralose micronized, (NF SPLENDA ®) | 0.40 g | 0.40 g |

| | | |
|---|---|---|
| 11 | NaOH | Qs to adjust pH > 6 |

Losartan potassium was dissolved in water and mixed with the rest of the ingredients. The pH was adjusted to 6 using a Sodium Hydroxide solution.

### Example 5: Losartan Solution

A solution of losartan suitable for oral administration was prepared having ingredients in the following proportions:

TABLE 4
Losartan solution for oral administration

| | | Formula Quantity |
|---|---|---|
| No | Ingredients | (g/100 mL) |
| 1. | Polyethylene Glycol 400, NF | 2.5 g |
| 2. | Propylene glycol, USP | 2.5 g |
| 3. | Methylparaben, NF | 0.18 g |
| 4. | Propylparaben, NF | 0.02 g |
| 5. | Sodium Phosphate, Monobasic USP | 0.114 g |
| 6. | Sodium Phosphate, Dibasic USP | 0.144 g |
| 7. | Losartan Potassium, USP | 1.00 g |
| 8. | Povidone, NF (Kollidon 90F) | 1.00 g |
| 9. | Hypromellose 2910, USP | 2.0 g |
| | (Methocel E50 Premium UV) | |
| 10. | Xanthan gum, NF | 0.10 g |
| 11. | Purified Water USP | QS |

Polyethylene glycol and Propylene Glycol were mixed, and Methylparaben and Propyl Paraben were added to the mixture to make the preservative phase. Phosphate buffer was prepared using Monobasic and Dibasic Sodium Phosphate. Losartan potassium was dissolved in water. The preservative phase and buffer phase (monobasic and dibasic sodium phosphate) were then added to make a clear solution. The remaining Ingredients were added with constant mixing using a homogenizer.

### Example 6: Losartan Suspension

A suspension of losartan suitable for oral administration was prepared having ingredients in the following proportions:

TABLE 5
Losartan suspension for oral administration:

| | | Quantity |
|---|---|---|
| No | Ingredients | (g/100 mL) |
| 1. | Polyethylene Glycol 400, NF | 2.5 g |
| 2. | Propylene glycol, USP | 2.5 g |
| 3. | Methylparaben, NF | 0.18 g |
| 4. | Propylparaben, NF | 0.02 g |
| 5. | Anhydrous Citric Acid, USP | 5.0 g |
| 6. | Vitamin E Polyethylene Glycol Succinate, NF | 2.0 g |
| 7. | Losartan Potassium, USP | 1.00 g |
| 8. | Povidone, NF (Kollidon 90F) | 1.0 g |
| 9. | Hypromellose 2910, USP (Methocel E50 Premium LV) | 1.0 g |
| 10. | Microcrystalline Cellulose & Carboxymethylcellulose | 1.0 g |
| | Sodium, NF (Avicel RC-591) | |
| 11. | Simethicone Emulsion, USP 30% | 1.0 g |
| 12. | Purified Water USP | QS |

Polyethylene glycol and Propylene Glycol were mixed, and Methylparaben and Propyl Paraben were added to the mixture.
Phosphate buffer was prepared using Monobasic and Dibasic Sodium Phosphate.
Citric acid was added to water to a 5% final concentration.
Losartan potassium was dissolved in water. The 5% citric acid solution was added to dissolve a portion of the API and adjust pH to 4.2.
The preservative phase and buffer phase were added.
The remaining Ingredients were added with constant mixing using a homogenizer. Citric acid was used to adjust the solution to a final pH of 4.2.

### Example 7: Losartan Powder for Solution

A solution of losartan suitable for oral administration was prepared from a composition having ingredients in the following proportions:

TABLE 6
Losartan solution for oral administration

| Sr. # | Ingredients | % w/w | gm/100 ml |
|---|---|---|---|
| 1 | Losartan Potassium | 33.33 | 1 |
| 2 | Sodium Benzoate | 1.67 | 0.05 |
| 3 | Sodium Citrate | 3.33 | 0.1 |
| 4 | Xanthan Gum | 1.67 | 0.05 |
| 6 | Citric acid | 1.67 | 0.05 |
| 7 | Sucralose | 1.67 | 0.05 |
| 8 | Sorbitol | 56.67 | 1.7 |

Ingredients 1-8 were mixed, then passed through a sieve having a mesh size of 20 (“20 #”), and further mixed using a blender. The resulting mixture can be combined with a suitable diluent to prepare a solution with is administrable as a pharmaceutical composition.

### Example 8: Losartan Powder for Solution

A solution of losartan suitable for oral administration was prepared from a composition having ingredients in the following proportions:

TABLE 7
Losartan solution for oral administration

| Sr.# | Ingredients | % w/w | gm/100 ml |
|---|---|---|---|
| 1 | Losartan Potassium | 33.33 | 1 |
| 2 | Potassium sorbate | 1.00 | 0.03 |
| 4 | Xanthan Gum | 1.67 | 0.05 |
| 6 | Dibasic Sodium/Calcium phosphate | 1.67 | 0.05 |
| 7 | Sucralose | 1.00 | 0.03 |
| 8 | Sorbitol | 61.33 | 1.84 |

Ingredients 1-8 were mixed, then passed through a 20 #sieve, and further mixed using a blender. The resulting mixture can be combined with a suitable diluent to prepare a solution with is administrable as a pharmaceutical composition.

### Example 9: Losartan Powder for suspension

A suspension of losartan suitable for oral administration was prepared from a composition having ingredients in the following proportions:

TABLE 8
Losartan Powder for oral administration

| Sr. # | Ingredients | mg |
|---|---|---|
| 1 | Losartan Potassium | 100 |
| 2 | Microcrystalline Cellulose | 105 |
| 3 | Lactose Monohydrate | 51 |
| 4 | Pregelatinized Starch 1500 | 41.9 |
| 5 | magnesium Stearate | 2.1 |
| 6 | Citric Acid | 0.75 |
| 7 | Sodium Citrate | 1 |
| 8 | Sucralose | 0.5 |
| 9 | Sorbitol | 97.75 |
| 10 | Xanthum gum | 5 |

Ingredients 1-10 were mixed and passed through a 20 #sieve, then further mixed using a rapid granulator. The powder mix was granulated with water and allowed to dry until the LOD was below 1%. The resulting mixture can be combined with a suitable diluent or suspending agent to prepare a suspension with is administrable as a pharmaceutical composition. For example, 40.5 mg of the mixture, which contains 10 mg of losartan, can be reconstituted in water to prepare a suitable dosage form.

### Example 10: Losartan Solution

A solution of losartan suitable for oral administration was prepared having ingredients in the following proportions:

TABLE 9
Losartan solution for oral administration

| Sr. # | Ingredients | % w/w | gm/100 ml |
|---|---|---|---|
| 1 | Losartan Potassium | 2.35 | 1 |
| 2 | Sodium Benzoate | 0.47 | 0.2 |
| 3 | Glycerine | 94.71 | 40.25 |
| 4 | Xanthan Gum | 0.59 | 0.25 |
| 5 | PVKK30 | 0.71 | 0.3 |
| 6 | Sucralose | 1.18 | 0.5 |

Losartan potassium was dissolved in water, and the remaining excipients were added with constant stirring using a homogenizer.

### Example 11: Losartan Solution

A solution of losartan suitable for oral administration was prepared having ingredients in the following proportions:

TABLE 10
Losartan solution for oral administration

| Sr. | | |
|---|---|---|
| # | Ingredient | g/1000 mL |
| 1 | Losartan Potassium, USP | 10 g |
| 2 | PEG 400 | 25 g |
| 3 | Propylene Glycol | 25 g |
| 4 | Monobasic sodium phosphate | 0.57 |
| | (anhydrous) | |
| 5 | Dibasic sodium phosphate | 0.72 |
| | (dried) | |
| 6 | Methyl Paraben | 1.8 g |
| 7 | Propyl Paraben | 0.2 g |
| 8 | Purified Water | QS to 1000 mL |

Polyethylene glycol and Propylene Glycol were mixed, and Methylparaben and Propyl Paraben were added to the mixture to make the preservative phase. Phosphate buffer was prepared using Monobasic and Dibasic Sodium Phosphate. Losartan potassium was dissolved in water. The preservative phase and buffer phase (monobasic and dibasic sodium phosphate) were then added to make a clear solution. Add purified water to total volume.

### Example 12: Losartan Solution

A solution of losartan suitable for oral administration was prepared having ingredients in the following proportions:

TABLE 11
Losartan solution for oral administration

| Sr.# | Ingredient | g/1000 mL |
|---|---|---|
| 1 | Losartan Potassium, USP | 10 g |
| 2 | Monobasic sodium phosphate | 0.57 |
| | (anhydrous) | |
| 3 | Dibasic sodium phosphate | 0.72 |
| | (dried) | |
| 4 | Methyl Paraben | 1.8 g |
| 5 | Propyl Paraben | 0.2 g |
| 6 | Water | q.s. to 1000 ml |

Preservatives (methylparaben, NF and propylparaben, NF) were dissolved in purified water at a temperature 80-85° C. Once dissolved, the content was cooled to 25° C.
Losartan (losartan potassium, USP) was added to the above solution once the temperature was between 25-30° C. and the drug was dissolved in water with preservatives.
Phosphate buffer was prepared using Monobasic and Dibasic Sodium Phosphate and added to the above solution.
Add purified water to total volume.

### Example 13: Losartan Suspension

A suspension of losartan suitable for oral administration was prepared having ingredients in the following proportions:

TABLE 12
Losartan suspension for oral administration

| Ingredient | Per 100 mL |
|---|---|
| Losartan Potassium, USP | 1.00 g |
| Methylparaben, NF | 0.18 g |
| Propylparaben, NF | 0.02 g |
| Purified Water, USP (q.s.) | 85.00 g + q.s. |
| Hydroxyethyl cellulose, NF (Natrosol | 1.2 g |
| 250 L Pharm) | |
| Xanthan Gum, NF (Xantural 180) | 0.10 g |
| Microcrystalline Cellulose and Carboxymethyl | 1.5 g |
| cellulose Sodium, NF (Avicel RC-591) | |
| Edetate Disodium, USP | 0.15 g |
| Simethicone Emulsion 30% USP (Q7-2587 | 1.00 g |
| Simethicone Emulsion 30% USP) | |
| Sucralose, NF (Splenda ®, Micronized | 0.40 g |
| (Powder) | |
| Monobasic Sodium phosphate (anhydrous) | 0.114 g |
| Disodium Phosphate (dried) | 0.144 g |

### Example 14: Method of Producing Suspension of Example 13

· Preservatives (methylparaben, NF and propylparaben, NF) were dissolved in purified water at a temperature 80-85° C. Once dissolved, the content was cooled to 25° C.
Losartan (losartan potassium, USP) was added to the above solution once the temperature was between 25-30° C. and the drug was dissolved in water with preservatives.
Phosphate buffer was prepared using Monobasic and Dibasic Sodium Phosphate and added to the above solution.
The viscosity modifying agents/thickening agents/suspending agents (hydroxyethyl cellulose (NATROSOL™ 250 L), NF and xanthan gum, NF (XANTURAL® 180)) were added to the composition of step 2 using high shear mixing.
Disodium edetate and simethicone emulsion 30% were added to the composition of step 4.
Sweetener was added and the composition was mixed for a few minutes.
Qs to batch volume with purified water and composition was mixed.

### Example 15: Analysis of Losartan Formulations Administered to Rabbits

60 male New Zealand white rabbits were split into Groups 1 to 6 of 10 random rabbits each.

5 mg of losartan potassium was administered orally to Group 1 as the formulation of Example 11 (a solution).

5 mg of losartan potassium was administered orally to Group 2 as the formulation of Example 12 (a solution).

5 mg of losartan potassium was administered orally to Group 3 as the formulation of Example 6 (a suspension).

5 mg of losartan potassium was administered orally to Group 4 as the formulation of Example 2 (a suspension).

5 mg of losartan potassium was administered orally to Group 5 as the formulation of Example 13 (a suspension).

5 mg of losartan potassium was administered orally to Group 6 as a suspension of COZAAR® prepared according to the instructions provided in COZAAR® label.

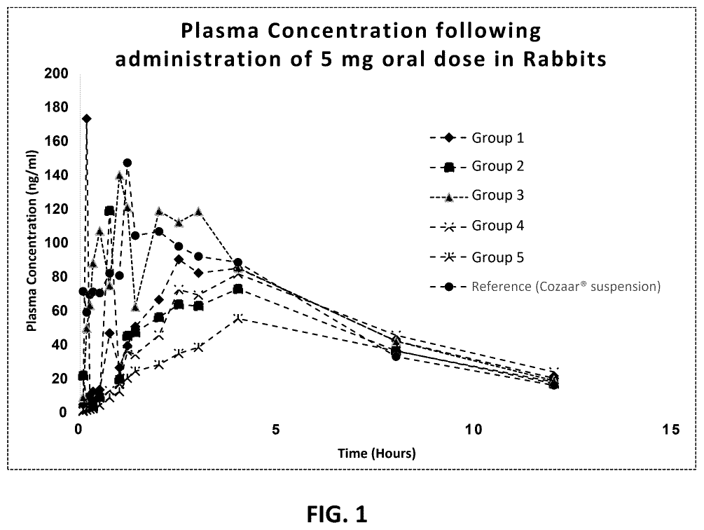

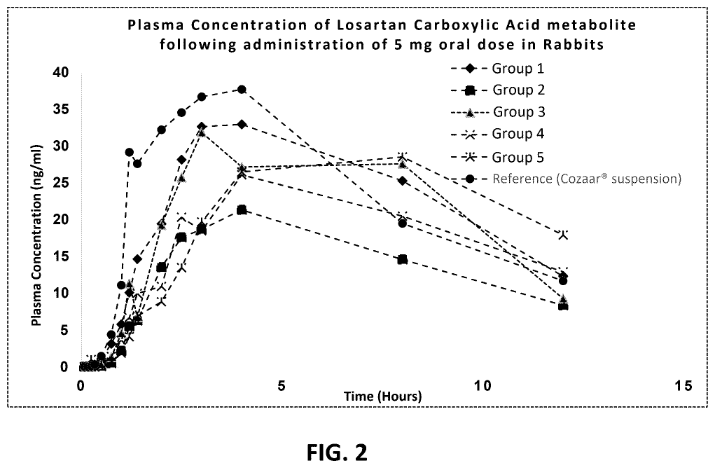

The plasma concentration of losartan over time following administration is depicted in FIG. 1. The plasma concentration of losartan carboxylic acid over time following administration is depicted in FIG. 2. Losartan carboxylic acid is a losartan metabolite. FIG. 1 demonstrates that all the administered formulations provide a therapeutically effective concentration of losartan over a period of at least ten hours. Multiple test formulations have a comparable Cₘₐₓ and AUC to that of the COZAAR® suspension.

Pharmacokinetic parameters for losartan are presented in Table 13 for each of the Groups 1 to 6. Average values are presented.

TABLE 13
Losartan Pharmacokinetic Parameters

| | | | | | | Group 6 |
|---|---|---|---|---|---|---|
| | | | | | | (compounded |
| | Group 1 | Group 2 | Group 3 | Group 4 | Group 5 | suspension; |
| | (solution) | (solution) | (suspension) | (suspension) | (suspension) | COZAAR ®) |
| Cₘₐₓ (ng/ml) | 181.05 | 173.73 | 212.11 | 93.78 | 57.03 | 205.76 |
| AUC_{0-24 hr} | 630.43 | 539.14 | 772.02 | 577.95 | 401.31 | 712.05 |
| (ng · h/ml) | | | | | | |
| AUC_{0-∞} | 763.32 | 682.8 | 976.0 | 902.8 | 528.7 | 880.5 |
| (ng · h/ml) | | | | | | |
| Tₘₐₓ (h) | 0.17 | 2.5 | 2.35 | 4 | 4 | 2 |
| T_{1/2} (h) | 4.53 | 3.51 | 4.81 | 5.58 | 5.23 | 3.41 |

The formulations of Groups 1 and 3 resulted in a Cₘₐₓ and an AUC_{0-∞} between about 80% to about 125% of the Cₘₐₓ and an AUC_{0-∞} for COZAAR® compounded suspension (Group 6). The formulation of Group 2 resulted in a Cₘₐₓ between about 80% to about 125% of the Cₘₐₓ of COZAAR® suspension but an AUC_{0-∞} less than 80% of the AUC_{0-∞} of COZAAR® suspension. The formulation of Group 4 resulted in an AUC_{0-∞} between about 80% to about 125% of the AUC_{0-∞} of COZAAR® suspension, but an Cₘₐₓ less than 80% of the Cₘₐₓ of COZAAR® suspension. The formulation of Group 5 resulted in a Cₘₐₓ and an AUC_{0-∞} below 80% of the Cₘₐₓ and an AUC_{0-∞} for COZAAR® suspension.

TABLE 14
Losartan Carboxylic Acid Pharmacokinetic Parameters
Pharmacokinetic parameters for losartan carboxylic acid are presented in Table 14
for each of the Groups 1 to 6. Average values are presented.

| | | | | | | Group 6 |
|---|---|---|---|---|---|---|
| | | | | | | (compounded |
| | Group 1 | Group 2 | Group 3 | Group 4 | Group 5 | suspension; |
| | (solution) | (solution) | (suspension) | (suspension) | (suspension) | COZAAR ®) |
| Cₘₐₓ (ng/ml) | 39.17 | 23.65 | 45.60 | 34.86 | 32.88 | 50.76 |
| AUC_{0-24 hr} | 267.27 | 162.73 | 250.30 | 209.43 | 245.43 | 278.64 |
| (ng · h/ml) | | | | | | |
| Tₘₐₓ (h) | 4 | 4 | 3.5 | 4 | 6 | 4 |

The data demonstrates that the test formulations of Groups 1-5 result in the pharmacokinetic profile of losartan metabolite comparable to that of COZAAR® suspension (Group 6).

### Example 16: Dissolution, Impurity, and Stability Analysis for the Formulation of Group 4

The formulation of Group 4 was tested for the presence of losartan Impurity D and losartan Impurity E under various conditions. As used in the Examples herein, “ACC” refers to accelerated conditions, in which the formulation was placed at 40° C. and 75% relative humidity (RH). “INT” refers to intermediate conditions, in which the formulation is placed at 30° C. and 65% RH. “CRT” refers to controlled room temperature, in which the formulation is placed at 25° C. and 60% RH.

TABLE 15
Analysis of the Formulation of Group 4 Under Various Conditions

| | | 1 Month | 1 Month | 1 Month | 2 Month | 2 Month |
|---|---|---|---|---|---|---|
| Test | Initial | ACC | IRT | CRT | INT | ACC |
| Assay for | 100.3% | 99.8% | 99.8% | 99.4% | 99.9% | 100.2 |
| Losartan | | | | | | |
| Potassium | | | | | | |
| Related | | | | | | |
| Substances: | | | | | | |
| Impurity D | ND | ND | ND | ND | ND | ND |
| Impurity E | ND | ND | ND | ND | ND | ND |
| Dissolution | 102.8% | 100.6% | 100.1% | 99.7% | 101.5% | 102.0% |
| in 30 mins | | | | | | |

ND: None detected

As Table 15 shows, the formulation of Group 4 retained over 99% of losartan potassium over all conditions tested, indicating purity and stability of the formulation. No Impurity D or Impurity E were detected under any of the conditions tested, further indicating both purity and stability of the losartan formulation. The same dissolution profile was observed in all conditions, further demonstrating stability. No color change was observed over time, further indicating purity and stability.

### Example 17: Impurity and Stability Analysis for the Formulation of Group 5

The formulation of Group 5 was tested for the presence of losartan Impurity D and losartan Impurity E under various conditions. Data are presented as wt/wt % relative to the theoretical initial amount of losartan potassium, which corresponds to about 10 mg/mL losartan.

TABLE 16
Analysis of the Formulation of Group 5 Under Various Conditions

| | | 20 days | 1 Month | 3 Month | 3 Month |
|---|---|---|---|---|---|
| Test | Initial | CRT | CRT | CRT | ACC |
| Assay for losartan | 102.1% | 98.7 | 97.6 | 102.3 | 98 |
| potassium | | | | | |
| Losartan Impurity | ND | ND | ND | ND | ND |
| D | | | | | |
| Losartan Impurity | ND | ND | ND | ND | 0.07 |
| E | | | | | |

ND: None detected

As Table 16 shows, the formulation of Group 5 retained at least 98% of losartan under all conditions tested, indicating stability and purity. Impurity D was not detectable and Impurity E was less than 0.1% wt/wt under all conditions tested, further indicating stability and purity.

### Example 18: Impurity and Stability Analysis for the Formulation of Example 5

The formulation of Example 5 was tested for the presence of losartan Impurity D and losartan Impurity E under various conditions. Data are presented as wt/wt % relative to the theoretical initial amount of losartan potassium.

TABLE 17
Analysis of the Formulation Example 5 Under Various Conditions

| Test | Initial | 48 days CRT |
|---|---|---|
| Assay for losartan | 99.50% | 99.90% |
| potassium | | |
| Losartan Impurity D | ND | ND |
| Losartan Impurity E | ND | ND |

ND: None detected

As Table 17 shows, the formulation of Example 5 retained over 99% of losartan under all conditions tested, indicating stability and purity. Impurity D was not detectable and Impurity E was less than the limits of quantification after 48 days at room temperature, further indicating stability and purity.

### Example 19: Exemplary Losaratan Formulation

Losartan potassium oral suspension 10 mg/mL (trade name ARBLI) was prepared. The composition is a white, translucent oral suspension with a peppermint odor, containing 10 mg/mL of losartan potassium and the following inactive ingredients: dibasic sodium phosphate, hypromellose, methyl paraben, monobasic sodium phosphate, natural peppermint flavor, polyethylene glycol, povidone, propyl paraben, propylene glycol, purified water, simethicone, sucralose, and xanthan gum.

Losartan potassium oral suspension 10 mg/mL is administered using an oral dosing syringe(s) or an oral dosing cup to measure the prescribed amount of medication.

### Adult Hypertension

The usual starting dose of losartan potassium oral suspension 10 mg/mL is 50 mg orally once daily. The dosage can be increased to a maximum dose of 100 mg orally once daily as needed to control blood pressure [see Example 30]. A starting dose of 25 mg is recommended for patients with possible intravascular depletion (e.g., on diuretic therapy).

### Pediatric Hypertension

The usual recommended starting dose of losartan potassium oral suspension 10 mg/mL is 0.7 mg per kg orally once daily (up to 50 mg total). Dosage should be adjusted according to blood pressure response. Doses above 1.4 mg per kg (or in excess of 100 mg) daily have not been studied in pediatric patients [See Examples 22, 28, and 30].

Losartan potassium oral suspension 10 mg/mL is not recommended in pediatric patients less than 2 years of age or in pediatric patients with estimated glomerular filtration rate less than 30 mL/min/1.73 m² [see Examples 26, 28, and 31].

Hypertensive Patients with Left Ventricular Hypertrophy

The usual starting dose is 50 mg of losartan potassium oral suspension 10 mg/ml orally once daily. Hydrochlorothiazide 12.5 mg daily should be added and/or the dose of losartan potassium oral suspension 10 mg/mL should be increased to 100 mg orally once daily followed by an increase in hydrochlorothiazide to 25 mg once daily based on blood pressure response [see Example 30].

### Nephropathy in Type 2 Diabetic Patients

The usual starting dose is 50 mg orally once daily. The dose should be increased to 100 mg orally once daily based on blood pressure response [see Example 31].

Dosage Modifications in Patients with Hepatic Impairment

In patients with mild-to-moderate hepatic impairment the recommended starting dose of losartan potassium oral suspension 10 mg/mL is 25 mg orally once daily. Losartan potassium oral suspension 10 mg/mL has not been studied in patients with severe hepatic impairment [see Examples 26 and 28].

### Example 20: Supply, Storage and Handling

Losartan potassium oral suspension 10 mg/mL is a white, translucent oral suspension that contains 10 mg of losartan potassium per mL. It is supplied as 165 mL in a high-density polyethylene (HDPE) bottle with a child-resistant cap and tamper-evident seal. Losartan potassium oral suspension 10 mg/mL should be shaken for 20 seconds prior to each use.

Losartan potassium oral suspension 10 mg/mL is stored at 20° C. to 25° C. (68° F. to 77° F.) with excursions permitted between 15° C. to 30° C. (59° F. to 86° F.) [see USP Controlled Room Temperature]. The container should be tightly closed and protected from light.

### Example 21: Contraindications

Losartan potassium oral suspension 10 mg/mL is contraindicated in patients who are hypersensitive to any component thereof. It is also contraindicated for co-administration with aliskiren in patients with diabetes.

### Example 22: Warnings and Precautions

### Fetal Toxicity

Losartan potassium oral suspension 10 mg/mL can cause fetal harm when administered to a pregnant woman. Use of drugs that act on the renin-angiotensin system during the second and third trimesters of pregnancy reduces fetal renal function and increases fetal and neonatal morbidity and death. Resulting oligohydramnios can be associated with fetal lung hypoplasia and skeletal deformations. Potential neonatal adverse effects include skull hypoplasia, anuria, hypotension, renal failure, and death. When pregnancy is detected, discontinue losartan potassium oral suspension 10 mg/mL as soon as possible [see Example 26].

### Hypotension in Volume- or Salt-Depleted Patients

In patients with an activated renin-angiotensin system, such as volume- or salt-depleted patients (e.g., those being treated with high doses of diuretics), symptomatic hypotension may occur after initiation of treatment with losartan potassium oral suspension 10 mg/mL. Volume correction or salt depletion is necessary prior to administration of losartan potassium oral suspension 10 mg/mL [see Example 19].

### Renal Function Deterioration

Changes in renal function including acute renal failure can be caused by drugs that inhibit the renin-angiotensin system and by diuretics. Patients whose renal function may depend in part on the activity of the renin-angiotensin system (e.g., patients with renal artery stenosis, chronic kidney disease, severe congestive heart failure, or volume depletion) may be at particular risk of developing acute renal failure on losartan potassium oral suspension 10 mg/mL. Renal function must be monitored periodically in these patients. Consider withholding or discontinuing therapy in patients who develop a clinically significant decrease in renal function on losartan potassium oral suspension 10 mg/mL [see Examples 25 and 26].

### Hyperkalemia

Serum potassium must be monitored periodically and treated appropriately. Dosage reduction or discontinuation of losartan potassium oral suspension 10 mg/mL may be required [see Example 23].

Concomitant use of other drugs that may increase serum potassium may lead to hyperkalemia [see Example 25].

### Example 23: Adverse Reactions

### Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

### Hypertension

Losartan potassium oral suspension 10 mg/mL has been evaluated for safety in more than 3,300 adult patients treated for essential hypertension and 4,058 patients/subjects overall. Over 1,200 patients were treated for over 6 months and more than 800 for over one year.

Treatment with losartan potassium oral suspension 10 mg/mL was well-tolerated with an overall incidence of adverse events similar to that of placebo. In controlled clinical trials, discontinuation of therapy for adverse events occurred in 2.3% of patients treated with losartan potassium oral suspension 10 mg/mL and 3.7% of patients given placebo. In 4 clinical trials involving over 1,000 patients on various doses (10 mg to 150 mg) of losartan potassium and over 300 patients given placebo, the adverse events that occurred in ≥2% of patients treated with losartan potassium oral suspension 10 mg/mL and more commonly than placebo were: dizziness (3% vs. 2%), upper respiratory infection (8% vs. 7%), nasal congestion (2% vs. 1%), and back pain (2% vs. 1%).

The following less common adverse reactions have been reported:

Blood and lymphatic system disorders: Anemia.

Psychiatric disorders: Depression.

Nervous system disorders: Somnolence, headache, sleep disorders, paresthesia, migraine.

Ear and labyrinth disorders: Vertigo, tinnitus.

Cardiac disorders: Palpitations, syncope, atrial fibrillation, CVA.

Respiratory, thoracic and mediastinal disorders: Dyspnea.

Gastrointestinal disorders: Abdominal pain, constipation, nausea, vomiting.

Skin and subcutaneous tissue disorders: Urticaria, pruritus, rash, photosensitivity.

Musculoskeletal and connective tissue disorders: Myalgia, arthralgia.

Reproductive system and breast disorders: Impotence.

General disorders and administration site conditions: Edema.

### Cough

Persistent dry cough (with an incidence of a few percent) has been associated with ACE-inhibitor use and in practice can be a cause of discontinuation of ACE-inhibitor therapy. Two prospective, parallel-group, double-blind, randomized, controlled trials were conducted to assess the effects of losartan on the incidence of cough in hypertensive patients who had experienced cough while receiving ACE-inhibitor therapy. Patients who had typical ACE-inhibitor cough when challenged with lisinopril, whose cough disappeared on placebo, were randomized to losartan 50 mg, lisinopril 20 mg, or either placebo (one study, n=97) or 25 mg hydrochlorothiazide (HCTZ) (n=135). The double-blind treatment period lasted up to 8 weeks. The incidence of cough is shown in Table 18 below.

TABLE 18

| Study 1* | HCTZ | Losartan | Lisinopril |
|---|---|---|---|
| Cough | 25% | 17% | 69% |
| Study 2^{†} | Placebo | Losartan | Lisinopril |
| Cough | 35% | 29% | 62% |

*Demographics = (89% Caucasian, 64% female)
^{†}Demographics = (90% Caucasian, 51% female)

These studies demonstrate that the incidence of cough associated with losartan therapy, in a population that all had cough associated with ACE-inhibitor therapy, is similar to that associated with hydrochlorothiazide or placebo therapy.

Cases of cough, including positive re-challenges, have been reported with the use of losartan in postmarketing experience (See Example 24).

Hypertensive Patients with Left Ventricular Hypertrophy

In the Losartan Intervention for Endpoint (LIFE) study, adverse reactions with losartan were similar to those reported previously for patients with hypertension.

### Nephropathy in Type 2 Diabetic Patients

In the Reduction of Endpoints in NIDDM with the Angiotensin II Receptor Antagonist Losartan (RENAAL) study involving 1,513 patients treated with losartan or placebo, the overall incidences of reported adverse events were similar for the two groups. Discontinuations of losartan because of side effects were similar to placebo (19% for losartan, 24% for placebo). The adverse events, regardless of drug relationship, reported with an incidence of ≥4% of patients treated with losartan and occurring with ≥2% difference in the losartan group vs. placebo on a background of conventional antihypertensive therapy, were asthenia/fatigue, chest pain, hypotension, orthostatic hypotension, diarrhea, anemia, hyperkalemia, hypoglycemia, back pain, muscular weakness, and urinary tract infection.

### Example 24: Postmarketing Experience

The following additional adverse reactions have been reported in postmarketing experience with losartan.

Because these reactions are reported voluntarily from a population of uncertain size, it is not always possible to estimate their frequency reliably or to establish a causal relationship to drug exposure:
Digestive: Hepatitis.
General Disorders and Administration Site Conditions: Malaise.
Hematologic: Thrombocytopenia.
Hypersensitivity: Angioedema, including swelling of the larynx and glottis, causing airway obstruction and/or swelling of the face, lips, pharynx, and/or tongue has been reported rarely in patients treated with losartan; some of these patients previously experienced angioedema with other drugs including ACE inhibitors. Vasculitis, including Henoch-Schönlein purpura, has been reported. Anaphylactic reactions have been reported.
Metabolic and Nutrition: Hyponatremia.
Musculoskeletal: Rhabdomyolysis.
Nervous System Disorders: Dysgeusia.
Skin: Erythroderma.

### Example 25: Drug Interactions

### Agents Increasing Serum Potassium

Co-administration of losartan with other drugs that raise serum potassium levels may result in hyperkalemia. Monitor serum potassium in such patients.

### Lithium

Losartan potassium oral suspension 10 mg/mL increases serum lithium concentrations. Lithium toxicity have been reported during concomitant administration of lithium with angiotensin II receptor antagonists. Serum lithium levels must be monitored during concomitant use.

### Non-Steroidal Anti-Inflammatory Drugs (NSAIDs) Including Selective Cyclooxygenase-2 Inhibitors (COX-2 Inhibitors)

In patients who are elderly, volume-depleted (including those on diuretic therapy), or with compromised renal function, co-administration of NSAIDs, including selective COX-2 inhibitors, with angiotensin II receptor antagonists (including losartan) may result in deterioration of renal function, including possible acute renal failure. These effects are usually reversible. Renal function must be periodically monitored in patients receiving losartan and NSAID therapy.

The antihypertensive effect of angiotensin II receptor antagonists, including losartan, may be attenuated by NSAIDs, including selective COX-2 inhibitors.

### Dual Blockade of the Renin-Angiotensin System (RAS)

Dual blockade of the RAS with angiotensin receptor blockers, ACE inhibitors, or aliskiren is associated with increased risks of hypotension, syncope, hyperkalemia, and changes in renal function (including acute renal failure) compared to monotherapy.

The Veterans Affairs Nephropathy in Diabetes (VA NEPHRON-D) trial enrolled 1,448 patients with type 2 diabetes, elevated urinary-albumin-to-creatinine ratio, and decreased estimated glomerular filtration rate (GFR 30 to 89.9 mL/min), randomized them to lisinopril or placebo on a background of losartan therapy and followed them for a median of 2.2 years. Patients receiving the combination of losartan and lisinopril did not obtain any additional benefit compared to monotherapy for the combined endpoint of decline in GFR, end stage renal disease, or death, but experienced an increased incidence of hyperkalemia and acute kidney injury compared with the monotherapy group.

In most patients no benefit has been associated with using two RAS inhibitors concomitantly. In general, avoid combined use of RAS inhibitors. Blood pressure, renal function, and electrolytes must be closely monitored in patients on losartan and other agents that affect the RAS.

Do not co-administer aliskiren with losartan in patients with diabetes. Avoid use of aliskiren with losartan in patients with renal impairment (GFR <60 mL/min).

### Example 26: Use in specific populations

### Pregnancy

### Risk Summary

Losartan potassium oral suspension 10 mg/mL can cause fetal harm when administered to a pregnant woman. Use of drugs that act on the renin-angiotensin system during the second and third trimesters of pregnancy reduces fetal renal function and increases fetal and neonatal morbidity and death (see Clinical Considerations in Example 26). Most epidemiologic studies examining fetal abnormalities after exposure to antihypertensive use in the first trimester have not distinguished drugs affecting the renin-angiotensin system from other antihypertensive agents. When pregnancy is detected, discontinue losartan as soon as possible.

The estimated background risk of major birth defects and miscarriage for the indicated population is unknown. All pregnancies have a background risk of birth defect, loss, or other adverse outcomes. In the U.S. general population, the estimated background risk of major birth defects and miscarriage in clinically recognized pregnancies is 2% to 4% and 15% to 20%, respectively.

### Clinical Considerations

Disease-associated Maternal and/or Embryo/Fetal Risk

Hypertension in pregnancy increases the maternal risk for pre-eclampsia, gestational diabetes, premature delivery, and delivery complications (e.g., need for cesarean section, post-partum hemorrhage). Hypertension increases the fetal risk for intrauterine growth restriction and intrauterine death. Pregnant women with hypertension should be carefully monitored and managed accordingly.

### Fetal/Neonatal Adverse Reactions

Oligohydramnios in pregnant women who use drugs affecting the renin-angiotensin system in the second and third trimesters of pregnancy can result in the following: reduced fetal renal function leading to anuria and renal failure, fetal lung hypoplasia, skeletal deformations, including skull hypoplasia, hypotension, and death. In the unusual case that there is no appropriate alternative to therapy with drugs affecting the renin-angiotensin system for a particular patient, apprise the mother of the potential risk to the fetus.

In patients taking losartan during pregnancy, perform serial ultrasound examinations to assess the intra-amniotic environment. Fetal testing may be appropriate, based on the week of gestation. If oligohydramnios is observed, discontinue losartan, unless it is considered lifesaving for the mother. Patients and physicians should be aware, however, that oligohydramnios may not appear until after the fetus has sustained irreversible injury.

Neonates with histories of in utero exposure to losartan for hypotension, oliguria, and hyperkalemia must be closely monitored. In neonates with a history of in utero exposure to losartan, if oliguria or hypotension occurs, support blood pressure and renal perfusion. Exchange transfusions or dialysis may be required as a means of reversing hypotension and replacing renal function.

### Data

### Animal Data

Losartan potassium was administered orally to rats during the period of late gestation through lactation (Gestation Day 15 through Lactation Day 20) at doses of 10, 25, and 100 mg/kg/day. Losartan potassium has been shown to produce adverse effects in rat fetuses and neonates, including decreased body weight, delayed physical and behavioral development, mortality and renal toxicity. With the exception of neonatal weight gain (which was affected at doses as low as 10 mg/kg/day), doses associated with these effects exceeded 25 mg/kg/day (approximately three times the maximum recommended human dose of 100 mg on a mg/m² basis). These findings are attributed to drug exposure in late gestation and during lactation. Significant levels of losartan and its active metabolite were shown to be present in rat fetal plasma during late gestation and in rat milk.

### Lactation

### Risk Summary

It is not known whether losartan is present in human milk, but significant levels of losartan and its active metabolite were shown to be present in rat milk Because of the potential for serious adverse reactions in breastfed infants, advise a lactating woman that breastfeeding is not recommended during the treatment with losartan and for 2 days after the last dose.

### Pediatric Use

Antihypertensive effects of losartan potassium oral suspension 10 mg/mL have been established in hypertensive pediatric patients aged 6 to 16 years. Safety and effectiveness have not been established in pediatric patients under the age of 6 or in pediatric patients with glomerular filtration rate <30 mL/min/1.73 m² [see Examples 19, 28 and 30]. Use of losartan potassium oral suspension 10 mg/mL is not recommended in children less than 2 years of age. It is not known whether post-natal use of losartan, before maturation of renal function is complete, has a long-term deleterious effect on the kidney.

### Geriatric Use

Of the total number of patients receiving losartan potassium oral suspension 10 mg/mL in controlled clinical studies for hypertension, 391 patients (19%) were 65 years and over, while 37 patients (2%) were 75 years and over. In a controlled clinical study for renal protection in type 2 diabetic patients with proteinuria, 248 patients (33%) were 65 years and over. In a controlled clinical study for the reduction in the combined risk of cardiovascular death, stroke and myocardial infarction in hypertensive patients with left ventricular hypertrophy, 2,857 patients (62%) were 65 years and over, while 808 patients (18%) were 75 years and over. No overall differences in effectiveness or safety were observed between these patients and younger patients, but greater sensitivity of some older individuals cannot be ruled out.

### Race

In the LIFE study, Black patients with hypertension and left ventricular hypertrophy treated with atenolol were at lower risk of experiencing the primary composite endpoint compared with Black patients treated with losartan (both cotreated with hydrochlorothiazide in the majority of patients). The primary endpoint was the first occurrence of stroke, myocardial infarction or cardiovascular death, analyzed using an intention-to-treat (ITT) approach. In the subgroup of Black patients (n=533, 6% of the LIFE study patients), there were 29 primary endpoints among 263 patients on atenolol (11%, 26 per 1,000 patient-years) and 46 primary endpoints among 270 patients (17%, 42 per 1,000 patient-years) on losartan. This finding could not be explained on the basis of differences in the populations other than race or on any imbalances between treatment groups. In addition, blood pressure reductions in both treatment groups were consistent between Black and non-Black patients. Given the difficulty in interpreting subset differences in large trials, it cannot be known whether the observed difference is the result of chance. However, the LIFE study provides no evidence that the benefits of losartan on reducing the risk of cardiovascular events in hypertensive patients with left ventricular hypertrophy apply to Black patients [see Example 30].

### Renal Impairment

Patients with renal insufficiency have elevated plasma concentrations of losartan and its active metabolite compared to subjects with normal renal function. No dose adjustment is necessary in patients with renal impairment unless a patient with renal impairment is also volume depleted [see Examples 19, 22 and 28].

### Hepatic Impairment

The recommended starting dose of losartan is 25 mg in patients with mild-to-moderate hepatic impairment. Following oral administration in patients with mild-to-moderate hepatic impairment, plasma concentrations of losartan and its active metabolite were, respectively, 5 times and 1.7 times those seen in healthy volunteers. Losartan has not been studied in patients with severe hepatic impairment [see Example 19 and 28].

### Example 27: Overdosage

Significant lethality was observed in mice and rats after oral administration of 1,000 mg/kg and 2,000 mg/kg, respectively, about 44 and 170 times the maximum recommended human dose on a mg/m² basis.

Limited data are available in regard to overdosage in humans. The most likely manifestation of overdosage would be hypotension and tachycardia; bradycardia could occur from parasympathetic (vagal) stimulation. If symptomatic hypotension should occur, supportive treatment should be instituted.

Neither losartan nor its active metabolite can be removed by hemodialysis.

### Example 28: Clinical Pharmacology

### Mechanism of Action

Angiotensin II [formed from angiotensin I in a reaction catalyzed by angiotensin converting enzyme (ACE, kininase II)] is a potent vasoconstrictor, the primary vasoactive hormone of the renin-angiotensin system, and an important component in the pathophysiology of hypertension. It also stimulates aldosterone secretion by the adrenal cortex. Losartan and its principal active metabolite block the vasoconstrictor and aldosterone-secreting effects of angiotensin II by selectively blocking the binding of angiotensin II to the AT₁ receptor found in many tissues, (e.g., vascular smooth muscle, adrenal gland). There is also an AT₂ receptor found in many tissues, but it is not known to be associated with cardiovascular homeostasis.

Neither losartan nor its principal active metabolite exhibits any partial agonist activity at the AT₁ receptor, and both have much greater affinity (about 1,000-fold) for the AT₁ receptor than for the AT₂ receptor. In vitro binding studies indicate that losartan is a reversible, competitive inhibitor of the AT₁ receptor. The active metabolite is 10 to 40 times more potent by weight than losartan and appears to be a reversible, noncompetitive inhibitor of the AT₁ receptor.

Neither losartan nor its principal active metabolite exhibits any partial agonist activity at the AT₁ receptor, and both have much greater affinity (about 1,000-fold) for the AT₁ receptor than for the AT₂ receptor. In vitro binding studies indicate that losartan is a reversible, competitive inhibitor of the AT₁ receptor. The active metabolite is 10 to 40 times more potent by weight than losartan and appears to be a reversible, noncompetitive inhibitor of the AT₁ receptor.

### Pharmacodynamics

Losartan inhibits the pressor effect of angiotensin II (as well as angiotensin I) infusions. A dose of 100 mg inhibits the pressor effect by about 85% at peak with 25% to 40% inhibition persisting for 24 hours. Removal of the negative feedback of angiotensin II causes a doubling to tripling in plasma renin activity and consequent rise in angiotensin II plasma concentration in hypertensive patients. Losartan does not affect the response to bradykinin, whereas ACE inhibitors increase the response to bradykinin. Aldosterone plasma concentrations fall following losartan administration. In spite of the effect of losartan on aldosterone secretion, very little effect on serum potassium was observed.

The effect of losartan is substantially present within one week, but in some studies, the maximal effect occurred in 3 to 6 weeks. In long-term follow-up studies (without placebo control), the effect of losartan appeared to be maintained for up to a year. There is no apparent rebound effect after abrupt withdrawal of losartan. There was essentially no change in average heart rate in losartan-treated patients in controlled trials.

### Pharmacokinetics

### Absorption

Following oral administration, losartan is well absorbed and undergoes substantial first-pass metabolism. The systemic bioavailability of losartan is approximately 33%. Mean peak concentrations of losartan and its active metabolite are reached in 1 hour and in 2 hours, respectively. While maximum plasma concentrations of losartan and its active metabolite are approximately equal, the AUC (area under the curve) of the metabolite is about 4 times as great as that of losartan. The pharmacokinetics of losartan and its active metabolite are linear with oral losartan doses up to 200 mg and do not change over time.

### Effect of Food

A high-fat high-calorie meal (937 calories, 56% fat) slows absorption of losartan and decreases the Cₘₐₓ of losartan and its active metabolite but has only minor effects on the AUC of losartan and its active metabolite.

### Distribution

The volume of distribution of losartan and the active metabolite is about 34 liters and 12 liters, respectively. Both losartan and its active metabolite are highly bound to plasma proteins, primarily albumin, with plasma free fractions of 1.3% and 0.2%, respectively. Plasma protein binding is constant over the concentration range achieved with recommended doses. Studies in rats indicate that losartan crosses the blood-brain barrier poorly, if at all.

### Elimination

Total plasma clearance of losartan and the active metabolite is about 600 mL/min and 50 mL/min, respectively, with renal clearance of about 75 mL/min and 25 mL/min, respectively. The terminal half-life of losartan and the active metabolite is about 2 hours and about 6 hours, respectively. Neither losartan nor its metabolite accumulates in plasma upon repeated once-daily dosing.

### Metabolism

Losartan is an orally active agent that undergoes substantial first-pass metabolism by cytochrome P450 enzymes. It is converted, in part, to an active carboxylic acid metabolite that is responsible for most of the angiotensin II receptor antagonism that follows losartan treatment. About 14% of an orally-administered dose of losartan is converted to the active metabolite. In addition to the active carboxylic acid metabolite, several inactive metabolites are formed. In vitro studies indicate that cytochrome P450 2C9 and 3A4 are involved in the biotransformation of losartan to its metabolites.

### Excretion

After single doses of losartan administered orally, about 4% of the dose is excreted unchanged in the urine and about 6% is excreted in urine as the active metabolite. Biliary excretion contributes to the elimination of losartan and its metabolites. Following oral 14C-labeled losartan, about 35% of radioactivity is recovered in the urine and about 60% in the feces. Following an intravenous dose of 14C-labeled losartan, about 45% of radioactivity is recovered in the urine and 50% in the feces.

### Specific Populations

### Geriatric Patients

Losartan pharmacokinetics have been investigated in the elderly (65 to 75 years). Plasma concentrations of losartan and its active metabolite are similar in elderly and young hypertensives. No dosage adjustment is necessary [see Example 19].

### Pediatric Patients

Pharmacokinetic parameters after multiple doses of losartan (average dose 0.7 mg/kg, range 0.36 to 0.97 mg/kg) to 25 hypertensive patients aged 6 to 16 years are shown in Table 2 below. Pharmacokinetics of losartan and its active metabolite were generally similar across the studied age groups and similar to historical pharmacokinetic data in adults. The principal pharmacokinetic parameters in adults and children are shown in the table below.

TABLE 19
Pharmacokinetic Parameters in Hypertensive
Adults and Children Age 6 to 16 Following Multiple Dosing

| | Adults given | | Age 6 to 16 given 0.7 | |
|---|---|---|---|---|
| | 50 mg once | | mg/kg once | |
| | daily for 7 days | | daily for 7 days | |
| | N = 12 | | N = 25 | |
| | | Active | | Active |
| | Parent | Metabolite | Parent | Metabolite |
| AUC₀₋₂₄ | 442 ± 173 | 1685 ± 452 | 368 ± 169 | 1866 ± 1076 |
| (ng · hr/mL)* | | | | |
| Cₘₐₓ (ng/ml)* | 224 ± 82 | 212 ± 73 | 141 ± 88 | 222 ± 127 |
| T_{1/2} (h)^{†} | 2.1 ± 0.70 | 7.4 ± 2.4 | 2.3 ± 0.8 | 5.6 ± 1.2 |
| T_{PEAK} (h)^{‡} | 0.9 | 3.5 | 2.0 | 4.1 |
| CL_{RE}N (mL/min) | 56 ± 23 | 20 ± 3 | 53 ± 33 | 17 ± 8 |

*Mean ± standard deviation
^{†}Harmonic mean and standard deviation
^{‡}Median

### Male and Female Patients

Losartan pharmacokinetics have been investigated in males and females. Plasma concentrations of losartan were about twice as high in female hypertensives as male hypertensives, but concentrations of the active metabolite were similar in males and females. No dosage adjustment is necessary [see Example 19].

### Racial or Ethnic Groups

Pharmacokinetic differences due to race have not been studied [see Example 26].

Patients with Renal Impairment

Following oral administration, plasma concentrations and AUCs of losartan and its active metabolite are increased by 50% to 90% in patients with mild (creatinine clearance of 50 to 74 mL/min) or moderate (creatinine clearance 30 to 49 mL/min) renal insufficiency. Renal clearance was reduced by 55% to 85% for both losartan and its active metabolite in patients with mild or moderate renal insufficiency. Neither losartan nor its active metabolite can be removed by hemodialysis [see Examples 22 and 26].

Patients with Hepatic Impairment

Following oral administration in patients with mild to moderate alcoholic cirrhosis of the liver, plasma concentrations of losartan and its active metabolite were, respectively, 5-times and about 1.7-times those in young male volunteers. Compared to normal subjects, the total plasma clearance of losartan in patients with hepatic insufficiency was about 50% lower and the oral bioavailability was about doubled. Use a starting dose of 25 mg for patients with mild to moderate hepatic impairment. Losartan has not been studied in patients with severe hepatic impairment [see Examples 19 and 26].

### Drug Interactions

No clinically significant drug interactions have been found in studies of losartan potassium with hydrochlorothiazide, digoxin, warfarin, cimetidine, and phenobarbital. However, rifampin has been shown to decrease the AUC of losartan and its active metabolite by 30% and 40%, respectively. Fluconazole, an inhibitor of cytochrome P450 2C9, decreased the AUC of the active metabolite by approximately 40%, but increased the AUC of losartan by approximately 70% following multiple doses. Conversion of losartan to its active metabolite after intravenous administration is not affected by ketoconazole, an inhibitor of P450 3A4. The AUC of active metabolite following oral losartan was not affected by erythromycin, an inhibitor of P450 3A4, but the AUC of losartan was increased by 30%.

The pharmacodynamic consequences of concomitant use of losartan and inhibitors of P450 2C9 have not been examined. Subjects who do not metabolize losartan to active metabolite have been shown to have a specific, rare defect in cytochrome P450 2C9. These data suggest that the conversion of losartan to its active metabolite is mediated primarily by P450 2C9 and not P450 3A4.

### Example 29: Nonclinical Toxicology

### Carcinogenesis, Mutagenesis, Impairment of Fertility

Losartan potassium was not carcinogenic when administered at maximally tolerated dosages to rats and mice for 105 and 92 weeks, respectively. Female rats given the highest dose (270 mg/kg/day) had a slightly higher incidence of pancreatic acinar adenoma. The maximally tolerated dosages (270 mg/kg/day in rats, 200 mg/kg/day in mice) provided systemic exposures for losartan and its pharmacologically active metabolite that were approximately 160 and 90 times (rats) and 30 and 15 times (mice) the exposure of a 50 kg human given 100 mg per day.

Losartan potassium was negative in the microbial mutagenesis and V-79 mammalian cell mutagenesis assays and in the in vitro alkaline elution and in vitro and in vivo chromosomal aberration assays. In addition, the active metabolite showed no evidence of genotoxicity in the microbial mutagenesis, in vitro alkaline elution, and in vitro chromosomal aberration assays.

Fertility and reproductive performance were not affected in studies with male rats given oral doses of losartan potassium up to approximately 150 mg/kg/day. The administration of toxic dosage levels in females (300/200 mg/kg/day) was associated with a significant (p<0.05) decrease in the number of corpora lutea/female, implants/female, and live fetuses/female at C-section. At 100 mg/kg/day only a decrease in the number of corpora lutea/female was observed. The relationship of these findings to drug-treatment is uncertain since there was no effect at these dosage levels on implants/pregnant female, percent post-implantation loss, or live animals/litter at parturition. In nonpregnant rats dosed at 135 mg/kg/day for 7 days, systemic exposure (AUCs) for losartan and its active metabolite were approximately 66 and 26 times the exposure achieved in man at the maximum recommended human daily dosage (100 mg).

### Example 30: Clinical Study-Hypertension

### Adult Hypertension

The antihypertensive effects of losartan were demonstrated principally in 4 placebo-controlled, 6-to-12-week trials of dosages from 10 to 150 mg per day in patients with baseline diastolic blood pressures of 95 to 115. The studies allowed comparisons of two doses (50 to 100 mg/day) as once-daily or twice-daily regimens, comparisons of peak and trough effects, and comparisons of response by gender, age, and race. Three additional studies examined the antihypertensive effects of losartan and hydrochlorothiazide in combination.

The 4 studies of losartan monotherapy included a total of 1,075 patients randomized to several doses of losartan and 334 to placebo. The 10- and 25-mg doses produced some effect at peak (6 hours after dosing) but small and inconsistent trough (24 hour) responses. Doses of 50, 100 and 150 mg once daily gave statistically significant systolic/diastolic mean decreases in blood pressure, compared to placebo in the range of 5.5 to 10.5/3.5 to 7.5 mmHg, with the 150-mg dose giving no greater effect than 50 mg to 100 mg. Twice-daily dosing at 50 to 100 mg/day gave consistently larger trough responses than once-daily dosing at the same total dose. Peak (6 h) effects were uniformly, but moderately, larger than trough effects, with the trough-to-peak ratio for systolic and diastolic responses 50% to 95% and 60% to 90%, respectively.

Addition of a low dose of hydrochlorothiazide (12.5 mg) to losartan 50 mg once daily resulted in placebo-adjusted blood pressure reductions of 15.5/9.2 mmHg.

Analysis of age, gender, and race subgroups of patients showed that men and women, and patients over and under 65, had generally similar responses. Losartan was effective in reducing blood pressure regardless of race, although the effect was somewhat less in Black patients (usually a low-renin population).

### Pediatric Hypertension

The antihypertensive effect of losartan was studied in one trial enrolling 177 hypertensive pediatric patients aged 6 to 16 years old. Children who weighed <50 kg received 2.5, 25 or 50 mg of losartan daily and patients who weighed ≥50 kg received 5, 50 or 100 mg of losartan daily. Children in the lowest dose group were given losartan in a suspension formulation [see Example 19]. The majority of the children had hypertension associated with renal and urogenital disease. The sitting diastolic blood pressure (SiDBP) on entry into the study was higher than the 95th percentile level for the patient's age, gender, and height. At the end of three weeks, losartan reduced systolic and diastolic blood pressure, measured at trough, in a dose-dependent manner. Overall, the two higher doses (25 to 50 mg in patients <50 kg; 50 to 100 mg in patients ≥50 kg) reduced diastolic blood pressure by 5 to 6 mmHg more than the lowest dose used (2.5 mg in patients <50 kg; 5 mg in patients≥50 kg). The lowest dose, corresponding to an average daily dose of 0.07 mg/kg, did not appear to offer consistent antihypertensive efficacy. When patients were randomized to continue losartan at the two higher doses or to placebo after 3 weeks of therapy, trough diastolic blood pressure rose in patients on placebo between 5 and 7 mmHg more than patients randomized to continuing losartan. When the low dose of losartan was randomly withdrawn, the rise in trough diastolic blood pressure was the same in patients receiving placebo and in those continuing losartan, again suggesting that the lowest dose did not have significant antihypertensive efficacy. Overall, no significant differences in the overall antihypertensive effect of losartan were detected when the patients were analyzed according to age (<, ≥12 years old) or gender. While blood pressure was reduced in all racial subgroups examined, too few non-White patients were enrolled to compare the dose-response of losartan in the non-White subgroup.

Hypertensive Patients with Left Ventricular Hypertrophy

The LIFE study was a multinational, double-blind study comparing losartan and atenolol in 9,193 hypertensive patients with ECG-documented left ventricular hypertrophy. Patients with myocardial infarction or stroke within six months prior to randomization were excluded. Patients were randomized to receive once daily losartan 50 mg or atenolol 50 mg. If goal blood pressure (<140/90 mmHg) was not reached, hydrochlorothiazide (12.5 mg) was added first and, if needed, the dose of losartan or atenolol was then increased to 100 mg once daily. If necessary, other antihypertensive treatments (e.g., increase in dose of hydrochlorothiazide therapy to 25 mg or addition of other diuretic therapy, calcium-channel blockers, alpha-blockers, or centrally acting agents, but not ACE inhibitors, angiotensin II antagonists, or beta-blockers) were added to the treatment regimen to reach the goal blood pressure.

Of the randomized patients, 4,963 (54%) were female and 533 (6%) were Black. The mean age was 67 with 5,704 (62%) age≥65. At baseline, 1,195 (13%) had diabetes, 1,326 (14%) had isolated systolic hypertension, 1,469 (16%) had coronary heart disease, and 728 (8%) had cerebrovascular disease. Baseline mean blood pressure was 174/98 mmHg in both treatment groups. The mean length of follow-up was 4.8 years. At the end of study or at the last visit before a primary endpoint, 77% of the group treated with losartan and 73% of the group treated with atenolol were still taking study medication. Of the patients still taking study medication, the mean doses of losartan and atenolol were both about 80 mg/day, and 15% were taking atenolol or losartan as monotherapy, while 77% were also receiving hydrochlorothiazide (at a mean dose of 20 mg/day in each group). Blood pressure reduction measured at trough was similar for both treatment groups but blood pressure was not measured at any other time of the day. At the end of study or at the last visit before a primary endpoint, the mean blood pressures were 144.1/81.3 mmHg for the group treated with losartan and 145.4/80.9 mmHg for the group treated with atenolol; the difference in systolic blood pressure (SBP) of 1.3 mmHg was significant (p<0.001), while the difference of 0.4 mmHg in diastolic blood pressure (DBP) was not significant (p=0.098).

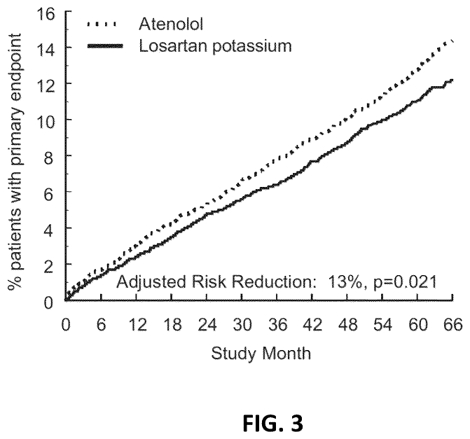

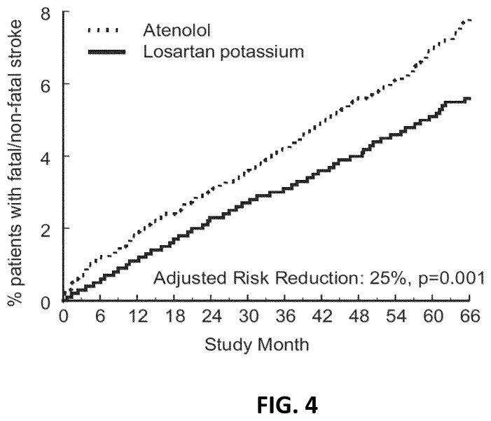

The primary endpoint was the first occurrence of cardiovascular death, nonfatal stroke, or nonfatal myocardial infarction. Patients with nonfatal events remained in the trial, so that there was also an examination of the first event of each type even if it was not the first event (e.g., a stroke following an initial myocardial infarction would be counted in the analysis of stroke). Treatment with losartan resulted in a 13% reduction (p=0.021) in risk of the primary endpoint compared to the atenolol group (see FIG. 3 and Table 20); this difference was primarily the result of an effect on fatal and nonfatal stroke. Treatment with losartan reduced the risk of stroke by 25% relative to atenolol (p=0.001) (see FIG. 4 and Table 20).

Table 20 shows the results for the primary composite endpoint and the individual endpoints. The primary endpoint was the first occurrence of stroke, myocardial infarction or cardiovascular death, analyzed using an ITT approach. The table shows the number of events for each component in two different ways. The Components of Primary Endpoint (as a first event) counts only the events that define the primary endpoint, while the Secondary Endpoints count all first events of a particular type, whether or not they were preceded by a different type of event.

TABLE 20

| | Losartan | | Atenolol | | Risk | 95% | |
|---|---|---|---|---|---|---|---|
| | N (%) | Rate | N (%) | Rate | Reduction † | CI | p-Value |
| Primary Composite | 508 (11) | 23.8 | 588 (13) | 27.9 | 13% | 2% to | 0.021 |
| Endpoint | | | | | | 23% | |
| Components of Primary Composite Endpoint (as a first event) | | | | | | | |
| Stroke (nonfatal) | 209 (5) | | 286 (6) | | | | |
| Myocardial infarction | 174 (4) | | 168 (4) | | | | |
| (nonfatal) | | | | | | | |
| Cardiovascular | 125 (3) | | 134 (3) | | | | |
| mortality | | | | | | | |

Secondary Endpoints (any time in study)

| | | | | | | | |
|---|---|---|---|---|---|---|---|
| Stroke (fatal/nonfatal) | 232 (5) | 10.8 | 309 (7) | 14.5 | 25% | 11% to | 0.001 |
| | | | | | | 37% | |
| Myocardial infarction | 198 (4) | 9.2 | 188 (4) | 8.7 | −7% | −13% to | 0.491 |
| fatal/nonfatal) | | | | | | 12% | |
| Cardiovascular | 204 (4) | 9.2 | 234 (5) | 10.6 | 11% | −7% to | 0.206 |
| mortality | | | | | | 27% | |
| Due to CHD | 125 (3) | 5.6 | 124 (3) | 5.6 | −3% | −32% to | 0.839 |
| | | | | | | 20% | |
| Due to Stroke | 40 (1) | 1.8 | 62 (1) | 2.8 | 35% | 4% to | 0.032 |
| | | | | | | 67% | |
| Other‡ | 39 (1) | 1.8 | 48 (1) | 2.2 | 16% | −28% to | 0.411 |

*Rate per 1,000 patient-years of follow-up
† Adjusted for baseline Framingham risk score and level of electrocardiogramtricular hypertrophy
‡Death due to heart failure, non-coronary vascular disease, pulmonary embolism, or a cardiovascular cause other than stroke or coronary heart disease

Although the LIFE study favored losartan over atenolol with respect to the primary endpoint (p=0.021), this result is from a single study and, therefore, is less compelling than the difference between losartan and placebo. Although not measured directly, the difference between losartan and placebo is compelling because there is evidence that atenolol is itself effective (vs. placebo) in reducing cardiovascular events, including stroke, in hypertensive patients.

Other clinical endpoints of the LIFE study were: total mortality, hospitalization for heart failure or angina pectoris, coronary or peripheral revascularization procedures, and resuscitated cardiac arrest. There were no significant differences in the rates of these endpoints between the losartan and atenolol groups.

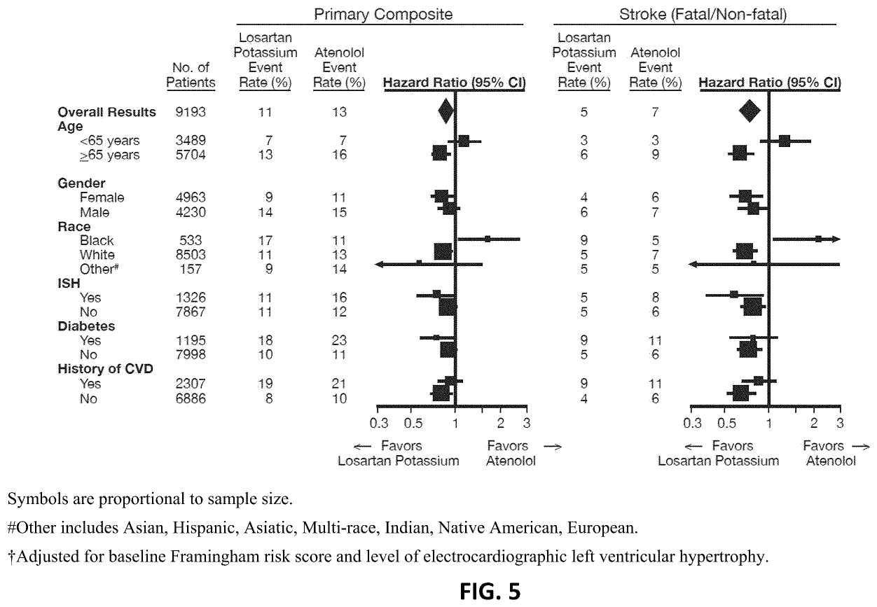

For the primary endpoint and stroke, the effects of losartan in patient subgroups defined by age, gender, race and presence or absence of isolated systolic hypertension (ISH), diabetes, and history of cardiovascular disease (CVD) are shown in FIG. 5. Subgroup analyses can be difficult to interpret and it is not known whether these represent true differences or chance effects.

### Example 31: Clinical Study—Nephropathy in Type 2 Diabetic Patients

The RENAAL study was a randomized, placebo-controlled, double-blind, multicenter study conducted worldwide in 1,513 patients with type 2 diabetes with nephropathy (defined as serum creatinine 1.3 to 3 mg/dL in females or males≤60 kg and 1.5 to 3 mg/dL in males >60 kg and proteinuria [urinary albumin to creatinine ratio ≥300 mg/g]).

Patients were randomized to receive losartan 50 mg once daily or placebo on a background of conventional antihypertensive therapy excluding ACE inhibitors and angiotensin II antagonists. After one month, investigators were instructed to titrate study drug to 100 mg once daily if the trough blood pressure goal (140/90 mmHg) was not achieved. Overall, 72% of patients received the 100-mg daily dose more than 50% of the time they were on study drug. Because the study was designed to achieve equal blood pressure control in both groups, other antihypertensive agents (diuretics, calcium-channel blockers, alpha- or beta-blockers, and centrally acting agents) could be added as needed in both groups. Patients were followed for a mean duration of 3.4 years.

The study population was diverse with regard to race (Asian 16.7%, Black 15.2%, Hispanic 18.3%, White 48.6%). Overall, 63.2% of the patients were men, and 66.4% were under the age of 65 years. Almost all of the patients (96.6%) had a history of hypertension, and the patients entered the trial with a mean serum creatinine of 1.9 mg/dL and mean proteinuria (urinary albumin/creatinine) of 1,808 mg/g at baseline.

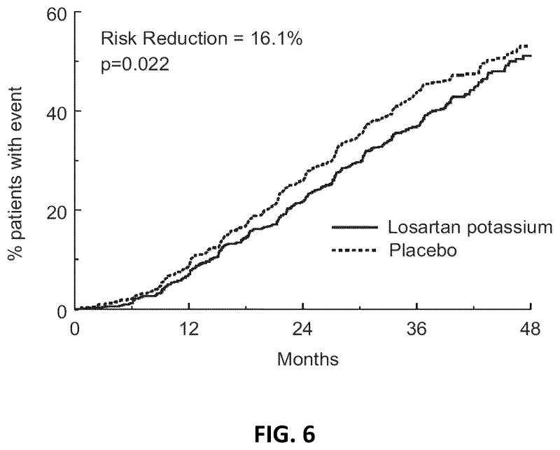

The primary endpoint of the study was the time to first occurrence of any one of the following events: doubling of serum creatinine, end-stage renal disease (ESRD) (need for dialysis or transplantation), or death. Treatment with losartan resulted in a 16% risk reduction in this endpoint (see FIG. 6 and Table 21). Treatment with losartan also reduced the occurrence of sustained doubling of serum creatinine by 25% and ESRD by 29% as separate endpoints, but had no effect on overall mortality (see Table 21).

The mean baseline blood pressures were 152/82 mmHg for losartan plus conventional antihypertensive therapy and 153/82 mmHg for placebo plus conventional antihypertensive therapy. At the end of the study, the mean blood pressures were 143/76 mmHg for the group treated with losartan and 146/77 mmHg for the group treated with placebo.

TABLE 21
Incidence of Primary Endpoint Events

| | Incidence | | Risk | | p- |
|---|---|---|---|---|---|
| | Losartan | Placebo | Reduction | 95% C.I. | Value |
| Primary Composite Endpoint | 43.5% | 47.1% | 16.1% | 2.3% to 27.9% | 0.022 |

Doubling of Serum Creatinine, ESRD and Death Occurring as a First Event

| | | |
|---|---|---|
| Doubling of Serum Creatinine | 21.6% | 26.0% |
| ESRD | 8.5% | 8.5% |
| Death | 13.4% | 12.6% |

Overall Incidence of Doubling of Serum Creatinine, ESRD and Death

| | | | | | |
|---|---|---|---|---|---|
| Doubling of Serum Creatinine | 21.6% | 26.0% | 25.3% | 7.8% to 39.4% | 0.006 |
| ESRD | 19.6% | 25.5% | 28.6% | 11.5% to 42.4% | 0.002 |
| Death | 21.0% | 20.3% | −1.7% | −26.9% to 18.6% | 0.884 |

The secondary endpoints of the study were change in proteinuria, change in the rate of progression of renal disease, and the composite of morbidity and mortality from cardiovascular causes (hospitalization for heart failure, myocardial infarction, revascularization, stroke, hospitalization for unstable angina, or cardiovascular death). Compared with placebo, losartan significantly reduced proteinuria by an average of 34%, an effect that was evident within 3 months of starting therapy, and significantly reduced the rate of decline in glomerular filtration rate during the study by 13%, as measured by the reciprocal of the serum creatinine concentration.

There was no significant difference in the incidence of the composite endpoint of cardiovascular morbidity and mortality.

The favorable effects of losartan were seen in patients also taking other anti-hypertensive medications (angiotensin II receptor antagonists and angiotensin converting enzyme inhibitors were not allowed), oral hypoglycemic agents and lipid-lowering agents.

For the primary endpoint and ESRD, the effects of losartan in patient subgroups defined by age, gender and race are shown in Table 22 below. Subgroup analyses can be difficult to interpret and it is not known whether these represent true differences or chance effects.

TABLE 22
Efficacy Outcomes within Demographic Subgroups

| | | Primary Composite Endpoint | | | ESRD | | |
|---|---|---|---|---|---|---|---|
| | | Losartan | Placebo | Hazard | Losartan | Placebo | Hazard |
| | No. of | Event Rate | Event Rate | Ratio (95% | Event Rate | Event Rate | Ratio (95% |
| | Patients | % | % | CI) | % | % | CI) |
| Overall | 1,513 | 43.5 | 47.1 | 0.84 (0.72, | 19.6 | 25.5 | 0.71 (0.58, |
| Results | | | | 0.98) | | | 0.89) |
| Age | | | | | | | |
| <65 years | 1,005 | 44.1 | 49.0 | 0.78 (0.65, | 21.1 | 28.5 | 0.67 (0.52, |
| | | | | 0.94) | | | 0.86) |
| ≥65 years | 508 | 42.3 | 43.5 | 0.98 (0.75, | 16.5 | 19.6 | 0.85 (0.56, |
| | | | | 1.28) | | | 1.28) |
| Gender | | | | | | | |
| Female | 557 | 47.8 | 54.1 | 0.76 (0.60, | 22.8 | 32.8 | 0.60 (0.44, |
| | | | | 0.96) | | | 0.83) |
| Male | 956 | 40.9 | 43.3 | 0.89 (0.73, | 17.5 | 21.5 | 0.81 (0.60, |
| | | | | 1.09) | | | 1.08) |
| Race | | | | | | | |
| Asian | 252 | 41.9 | 54.8 | 0.66 (0.45, | 18.8 | 27.4 | 0.63 (0.37, |
| | | | | 0.95) | | | 1.07) |
| Black | 230 | 40.0 | 39.0 | 0.98 (0.65, | 17.6 | 21.0 | 0.83 (0.46, |
| | | | | 1.50) | | | 1.52) |
| Hispanic | 277 | 55.0 | 54.0 | 1.00 (0.73, | 30.0 | 28.5 | 1.02 (0.66, |
| | | | | 1.38) | | | 1.59) |
| White | 735 | 40.5 | 43.2 | 0.81 (0.65, | 16.2 | 23.9 | 0.60 (0.43, |
| | | | | 1.01) | | | 0.83) |

1. A method for treating hypertension to lower blood pressure in an adult or pediatric subject greater than 6 years old, comprising administering to the subject an oral suspension, wherein the oral suspension comprises:
i.) about 1 mg/mL to about 10 mg/mL of losartan or a pharmaceutically acceptable salt thereof,
ii.) about 0.5 mg/mL to about 1.5 mg/mL of a suspending agent selected from the group consisting of hydroxyethylcellulose, methylcellulose, hydroxymethylcellulose, hydroxypropylmethylcellulose, microcrystalline cellulose, sodium carboxymethylcellulose, xanthan gum, acacia, an alginate, and guar gum, or a combination thereof,
iii.) a pH modifying agent, and
iv.) about 0.1 mg/mL to about 3 mg/mL of a preservative,
wherein the oral suspension has a pH of about 7, and
wherein the oral suspension has the following characteristics after storage at room temperature and 60% relative humidity;
after 12 months of storage, the oral suspension comprises less than about 0.5 wt/wt % losartan impurity D and losartan impurity E, relative to the weight of losartan in the oral suspension, and
after 12 months of storage, the oral suspension comprises at least about 95% of the losartan that was in the oral suspension prior to storage.

2. A method for reducing the risk of stroke in a subject with hypertension and left ventricular hypertrophy comprising administering to the subject an oral suspension, wherein the oral suspension comprises:
i.) about 1 mg/mL to about 10 mg/mL of losartan or a pharmaceutically acceptable salt thereof,
ii.) about 0.5 mg/mL to about 1.5 mg/mL of a suspending agent selected from the group consisting of hydroxyethylcellulose, methylcellulose, hydroxymethylcellulose, hydroxypropylmethylcellulose, microcrystalline cellulose, sodium carboxymethylcellulose, xanthan gum, acacia, an alginate, and guar gum, or a combination thereof,
iii.) a pH modifying agent, and
iv.) about 0.1 mg/mL to about 3 mg/mL of a preservative,
wherein the oral suspension has a pH of about 7, and
wherein the oral suspension has the following characteristics after storage at room temperature and 60% relative humidity;
after 12 months of storage, the oral suspension comprises less than about 0.5 wt/wt % losartan impurity D and losartan impurity E, relative to the weight of losartan in the oral suspension, and
after 12 months of storage, the oral suspension comprises at least about 95% of the losartan that was in the oral suspension prior to storage.

3. A method for treating diabetic nephropathy with an elevated serum creatinine and proteinuria in a subject with type 2 diabetes and a history of hypertension comprising administering to the subject an oral suspension, wherein the oral suspension comprises:
i.) about 1 mg/mL to about 10 mg/mL of losartan or a pharmaceutically acceptable salt thereof,
ii.) about 0.5 mg/mL to about 1.5 mg/mL of a suspending agent selected from the group consisting of hydroxyethylcellulose, methylcellulose, hydroxymethylcellulose, hydroxypropylmethylcellulose, microcrystalline cellulose, sodium carboxymethylcellulose, xanthan gum, acacia, an alginate, and guar gum, or a combination thereof,
iii.) a pH modifying agent, and
iv.) about 0.1 mg/mL to about 3 mg/mL of a preservative,
wherein the oral suspension has a pH of about 7, and
wherein the oral suspension has the following characteristics after storage at room temperature and 60% relative humidity;
after 12 months of storage, the oral suspension comprises less than about 0.5 wt/wt % losartan impurity D and losartan impurity E, relative to the weight of losartan in the oral suspension, and
after 12 months of storage, the oral suspension comprises at least about 95% of the losartan that was in the oral suspension prior to storage.

4. The method of claim 1, wherein the oral suspension comprises 10 mg/mL losartan potassium, dibasic sodium phosphate, hypromellose, methyl paraben, monobasic sodium phosphate, natural peppermint flavor, polyethylene glycol, povidone, propyl paraben, propylene glycol, purified water, simethicone, sucralose, and xanthan gum.

5. The method of claim 1, comprising administering to the subject a starting dose of 50 mg of losartan or a pharmaceutically acceptable salt thereof once daily.

6. The method of claim 1, wherein the subject is administered a total daily dose of 100 mg of losartan or a pharmaceutically acceptable salt thereof.

7. The method of claim 1, comprising administering to the subject a starting dose of 25 mg, wherein the subject has intravascular depletion.

8. The method of claim 1, comprising administering a starting dose of 0.7 mg per kg, and the subject is a pediatric subject.

9. The method of claim 2, further comprising co-administering to the subject hydrochlorothiazide at 12.5 mg once daily.

10. The method of claim 6, further comprising co-administering to the subject hydrochlorothiazide at 25 mg once daily.

11. The method of claim 3, wherein the subject is administered a total daily dose of 100 mg of losartan or a pharmaceutically acceptable salt thereof.

12. The method of claim 1, comprising administering to the subject a starting dose of 25 mg once daily, wherein the subject has mild-to-moderate hepatic impairment.

13. The method of claim 1, wherein the subject is not pregnant and/or not a breastfeeding woman.

14. The method of claim 1, wherein the oral suspension is not co-administered with
(a) a drug or supplement that raises serum potassium levels,
(b) a drug that increases serum lithium concentrations,
(c) an NSAID (d)
(d) an ACE inhibitor (e)
(e) a drug that acts as a renin-angiotensin system (RAS) and/or angiotensin receptor blockers
(f) aliskiren
(g) an agent that affects the renin-angiotensin system (RAS)
(h) an inhibitor of cytochrome P450.

15. The method of claim 1, wherein the adult subject is 65 years and over.

16. The method of claim 1, wherein the pediatric subject is age 6 to 16.

17. (canceled)

18. The method of claim 1, wherein the subject has renal impairment.

19. The method of claim 1, wherein the oral suspension comprises 10 mg/ml of losartan potassium.

20. The method of claim 2, wherein the oral suspension comprises 10 mg/ml of losartan potassium.

21. The method of claim 3, wherein the oral suspension comprises 10 mg/ml of losartan potassium.
